(19) United States
(12) Patent Application Publication
(10) Pub. No.: US 2020/0165225 A1
(43) Pub. Date: May 28, 2020

(54) BACE1 INHIBITORS FOR TREATMENT OF ALZHEIMER'S DISEASE

(71) Applicant: NantBio, Inc., Culver City, CA (US)

(72) Inventors: Chunlin TAO, Newport Coast, CA (US); Tulay POLAT, Tustin, CA (US); Chengzhi YU, San Diego, CA (US)

(73) Assignee: NantBio, Inc., Culver City, CA (US)

(21) Appl. No.: 16/626,717

(22) PCT Filed: May 8, 2018
(86) PCT No.: PCT/US2018/031490

§ 371 Date: Dec. 26, 2019

Related U.S. Application Data
(60) Provisional application No. 62/525,962, filed on Jun. 28, 2017.

(51) Int. Cl. C07D 401/14; C07D 401/04; C07D 403/10

(52) U.S. Cl. CPC C07D 401/14; C07D 403/10; C07D 401/04

(57) ABSTRACT

Compounds, pharmaceutical compositions containing the same useful in the treatment of Alzheimer's disease are disclosed. Methods of preparing the compounds and compositions and use thereof are also disclosed.

### FIELD OF THE INVENTION

The present invention is related to compounds useful in the treatment of Alzheimer's disease, synthesis of such compounds and methods of treatment of the disease using the compounds.

### BACKGROUND OF THE INVENTION

Alzheimer's disease (AD) is one of the most common types of dementia. Subjects afflicted with the disease typically present with a slowly progressive cognitive decline.

Cognition can be generally described as including at least three different components: attention, learning, and memory. Each of these components and their respective levels affect the overall level of a subject's cognitive ability. For instance, while Alzheimer's disease patients suffer from a loss of overall cognition and thus deterioration of each of these characteristics, it is the loss of memory that is most often associated with the disease. Other conditions include general dementias associated with other neurological diseases, and aging.

Two key characteristics of AD are the accumulation of extracellular deposits containing aggregated amyloid-β (Aβ) peptide and neuronal synaptic loss in specific brain regions.

BACE1 (β-site amloid precursor protein cleaving enzyme 1) is a key enzyme involved in processing of amyloid-β protein precursor (AβPP) and ultimately to the production of Aβ species and AD pathology. BACE1 (also called β-secretase) has become a focus of research and development since its discovery, and has perhaps surpassed γ-secretase as the most promising target for pharmaceutical research on AD therapeutics.

Over the years there has been extensive research in the field of improving cognitive abilities of patients having reduced cognitive abilities. For example, treatments have been suggested for improving memory in subjects with Alzheimer's disease. While transient improvements have been observed with some patients, there continues to be a need for compounds which improve overall cognition and reduce memory loss. The present invention addresses this need.

### DETAIL DESCRIPTION OF THE INVENTION

The compounds and methods of treatment described herein are beneficial in the treatment of Alzheimer's disease and other neurodegenerative diseases, as well as, provide for improved cognitive enhancement. In accordance therewith, the present invention is related to compounds corresponding to general Formula (I)

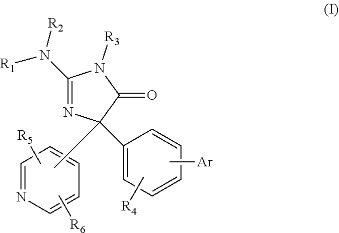

or a pharmaceutically acceptable salt thereof, wherein:
Ar is one of phenyl, pyridyl, pyrimidyl, pyrazinyl, imidazolyl, pyrazolyl, thiazolyl, oxazolyl, and isoxazolyl, wherein the Ar is optionally substituted with one or more substituent selected from among halogen, CN, C₁-C₄ alkyl, C₂-C₄ alkenyl, C₂-C₄ alkynyl, C₁-C₄ fluoroalkyl and C₁-C₄ alkoxy;
R₁ and R₂ are independently hydrogen or a C₁-C₄ alkyl;
R₃ is hydrogen or a C₁-C₆ alkyl, which is optionally substituted with halogen, hydroxy, cyano, amino, —COOH or oxo;
R₄ is one of hydrogen, halogen, CN, OH, NR₇R₈, CONR₇R₈, C₁-C₄ alkyl, C₂-C₄ alkenyl, C₂-C₄ alkynyl, C₃-C₄ aryl or heteroaryl, (C₃-C₄ cycloalkyl)C₁-C₄ alkyl, C₁-C₄ haloalkyl, C₁-C₄ alkoxy, C₁-C₄ alkylthio, C₂-C₄ alkanoyl, C₁-C₄ alkoxycarbonyl, C₂-C₄ alkanoyloxy, C₁-C₄ alkylsulfonyl, mono- and di-(C₁-C₄ alkyl) sulfonamido, and mono- and
di-(C₁-C₄ alkyl)aminocarbonyl, each of which is optionally substituted with from 1 to 4 substituents independently chosen from halogen, hydroxy, cyano, amino, —COOH and oxo;
R₅ is one of methyl, ethyl, monofluoromethyl, difluoromethyl, trifluormethyl, difluoroethyl, methoxy, ethoxy, methoxymethyl, and CN;
R₆ is one of hydrogen, halogen, CN, OR₇, amino, COOH, and C₁-C₄ alkyl, each of which is optionally substituted with from 1 to 4 substituents independently chosen from the group consisting of halogen, hydroxy, cyano, amino, —COOH and oxo; and
R₇ and R₈ are independently selected from among hydrogen, C₁-C₄ alkyl, C₂-C₄alkenyl, and C₂-C₄ alkynyl.

In a further embodiment, Ar is an optionally substituted heteroaryl group. Representative heteroaryl groups include pyridine, thiophene, thiazole, thiadiazole, furan, oxazole, oxadiazole, pyrrole, pyrazole, imidazole, triazole, oxathiole, isoxazole, oxazole, oxatriazole, dioxazole, oxathiazole, tetrazole, pyridazine, pyrimidine, pyrazine, triazine, oxazine, oxathiazine, or oxadiazine. The heteroaryl group may be unsubstituted or substituted with alkyl, alkoxy, trifluoroalkyl, trifluoroalkoxy, amino, halogen, hydroxyl, or CN, or forms an N-oxide. For example Ar may be an optionally substituted pyridine or pyrimidine group.

In another embodiment, Ar is a phenyl group optionally substituted with CN, OCF₃ or halogen.

For purposes of the present invention, the following shall be understood:

The term “alkyl,” as a group, refers to a straight or branched hydrocarbon chain containing the specified number of carbon atoms. When the term “alkyl” is used without reference to a number of carbon atoms, it is to be understood to refer to a C₁-C₄ alkyl. For example, C₁-C₄ alkyl refers to a straight or branched alkyl containing at least 1, and at most 4, carbon atoms. Examples of “alkyl” as used herein include, but are not limited to, methyl, ethyl, n-propyl, n-butyl, n-pentyl, i-butyl, i-propyl, t-butyl, hexyl, heptyl, octyl, nonyl and decyl. Preferably, the alkyl group is a lower alkyl of from about 1 to 7 carbons, yet more preferably about 1 to 4 carbons. The alkyl group can be substituted or unsubstituted.

The term “substituted alkyl” as used herein denotes alkyl radicals wherein at least one hydrogen is replaced by one or more substituents such as, but not limited to, hydroxy, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition is pertinent whether the term is applied to a substituent itself or to a substituent of a substituent.

The term “alkoxy” refers to an alkyl group of an indicated number of carbon atoms attached to the parent molecular moiety through an oxygen bridge. Examples of alkoxy groups include, for example, methoxy, ethoxy, propoxy and isopropoxy. When the term “alkoxy” is used without reference to a number of carbon atoms, it is to be understood to refer to a C1-C10 alkoxy in which the alkyl group can be straight, branched, saturated or unsaturated alkyls containing at least 1, and at most 10, carbon atoms. Preferably, it is a lower alkoxy of from about 1 to 4 carbons.

The term “halo” or “halogen” refers to fluoro, chloro, bromo, and iodo.

The term “substituted” refers to adding or replacing one or more atoms contained within a functional group or compound with one of the moieties from the group of halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C1-6 alkylcarbonylalkyl, aryl, and amino groups.

The phrase “pharmaceutically acceptable” refers to molecular entities and compositions that are generally regarded as safe. In particular, pharmaceutically acceptable carriers used in the practice of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the appropriate governmental agency or listed in the U.S. Pharmacopoeia or other generally recognized pharmacopoeia for use in humans.

The term “salts” can include acid addition salts or addition salts of free bases. Suitable pharmaceutically acceptable salts include, but are not limited to, hydrochloride, hydrobromide, sulfate, phosphate; sodium, potassium etc. as will be understood by those of ordinary skill. (see, for example, Berge, et al. “Pharmaceutical Salts,” J. Pharm. Sci. 1977; 66:1), the contents of which are incorporated herein by reference.

The term “about,” unless otherwise indicated, refers to ±10% of the given value.

The term “treating” includes: (1) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a subject to be treated is either statistically significant or at least perceptible to the subject or to the physician.

The terms “subject” and “patient” refer primarily to humans but can also include other mammals.

“Effective amount” means an amount of a composition of the present invention sufficient to result in the desired therapeutic response. The therapeutic response can be any response that a user (e.g., a clinician) will recognize as an effective response to the therapy. The therapeutic response will generally be amelioration of the typical symptoms of Alzheimer's disease. It is further within the competency of one skilled in the art to determine appropriate treatment duration, appropriate doses, and any potential combination treatments, based upon an evaluation of therapeutic response.

In some embodiments, the compound of Formula (I) is selected from among:

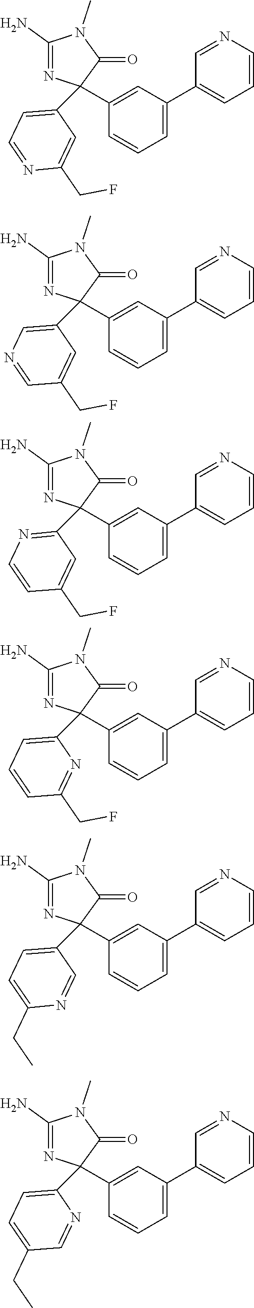

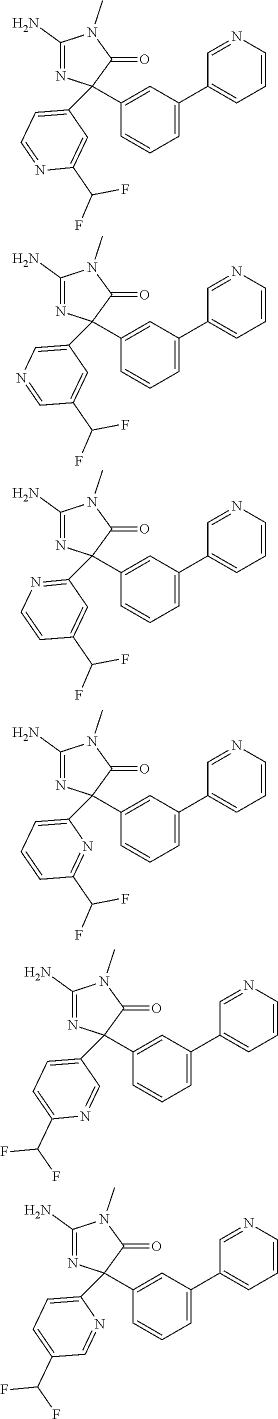

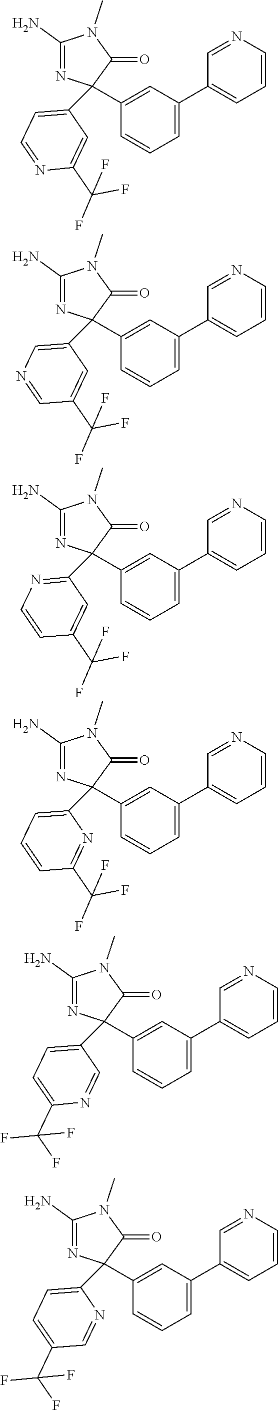

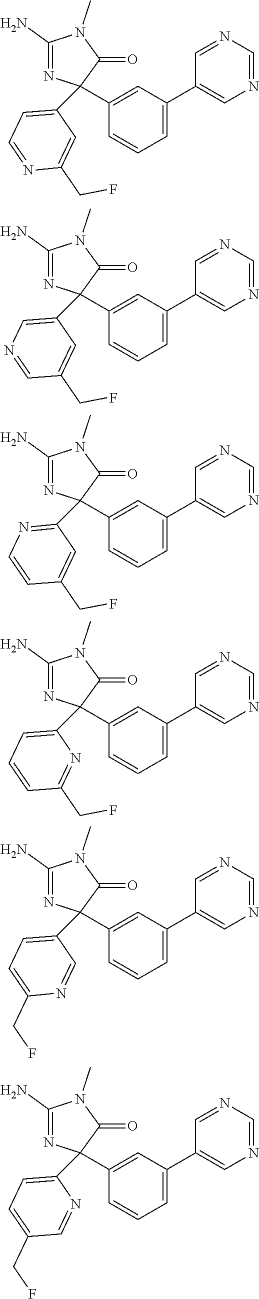

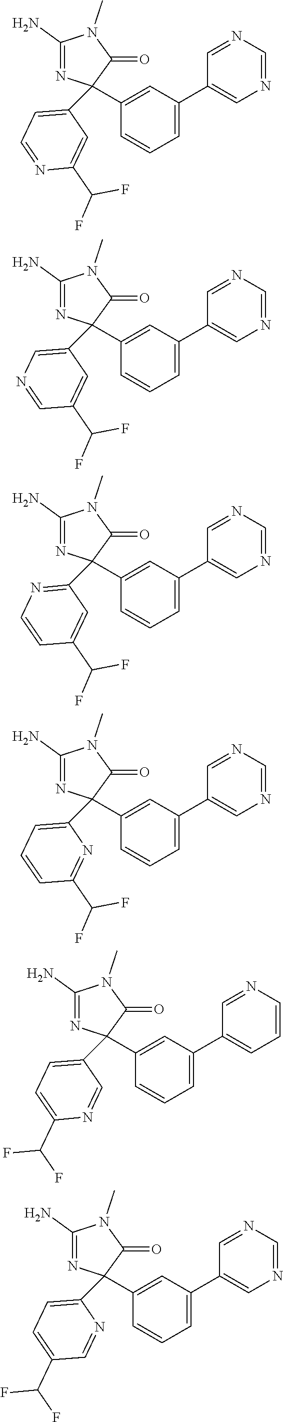

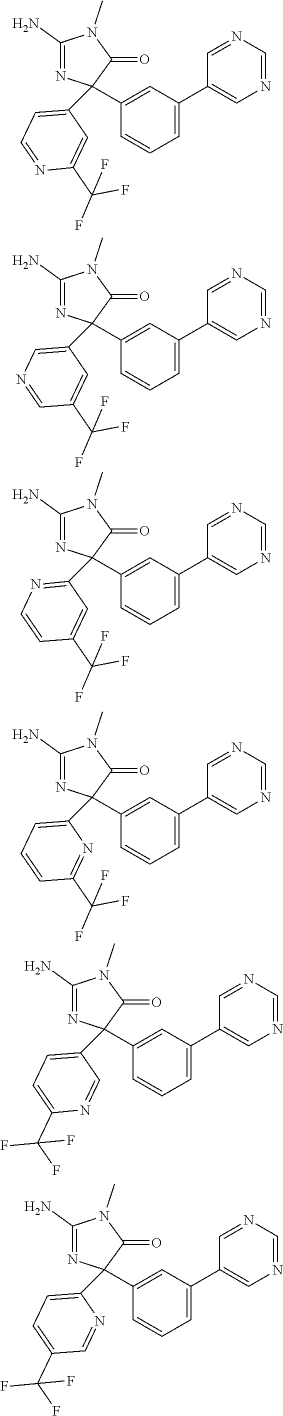

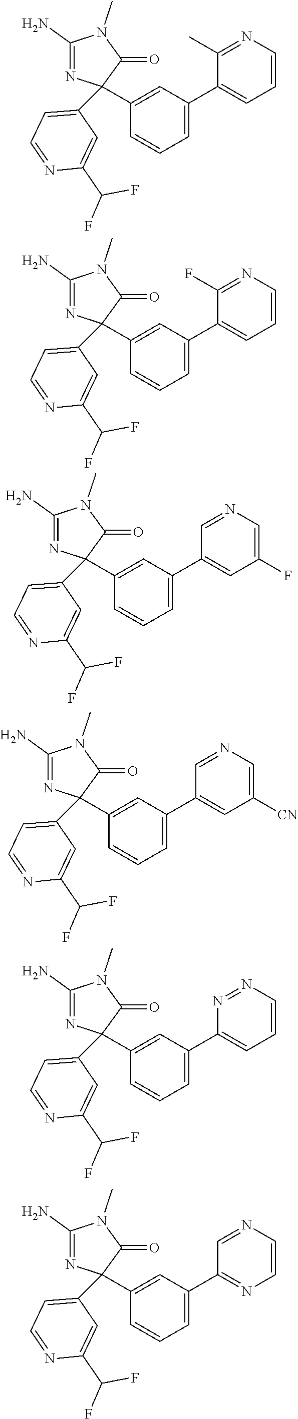

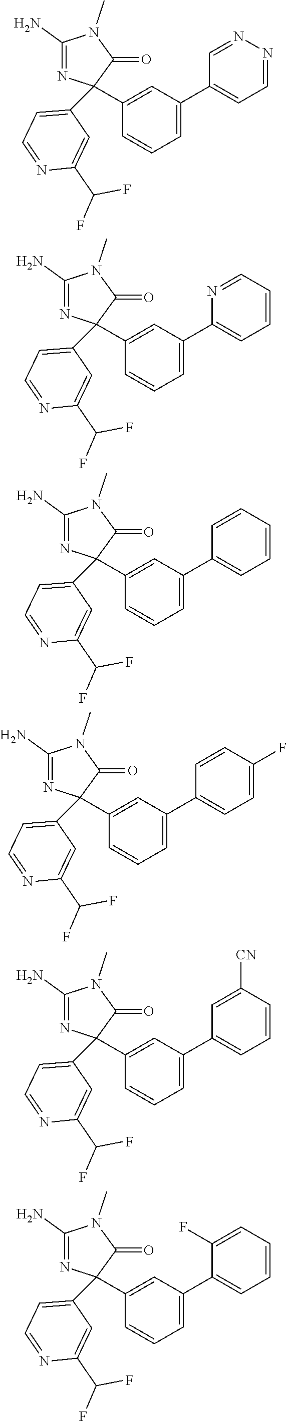

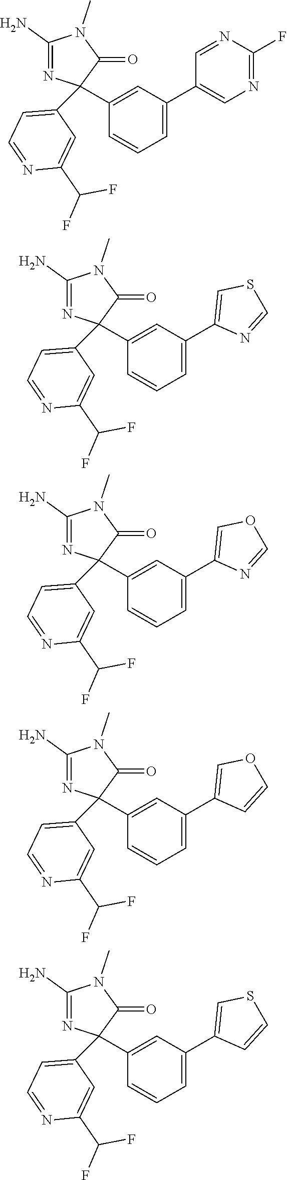

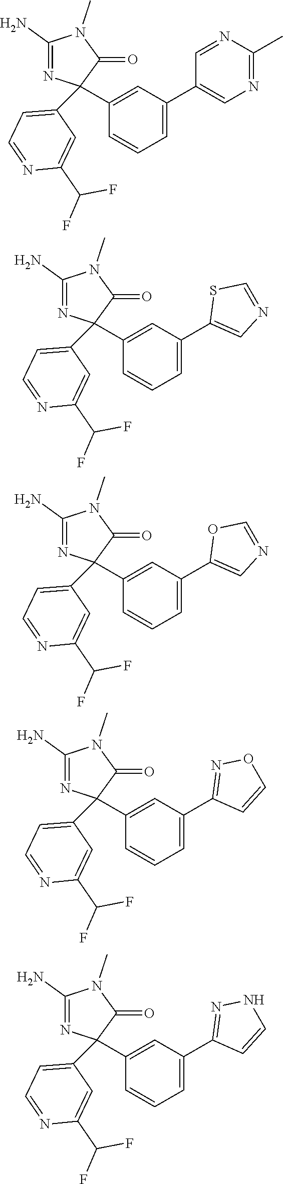

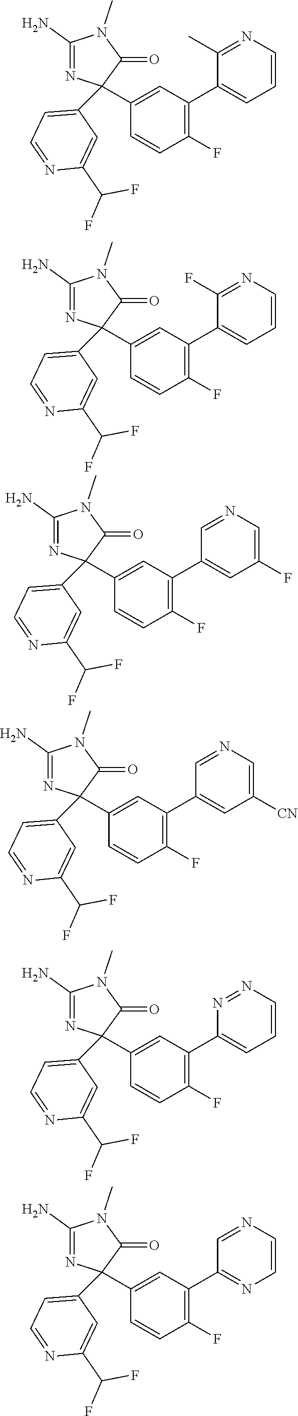

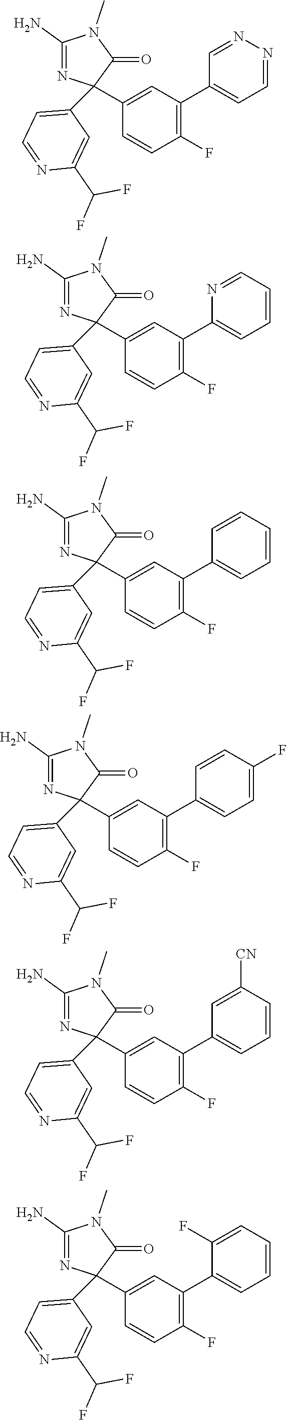

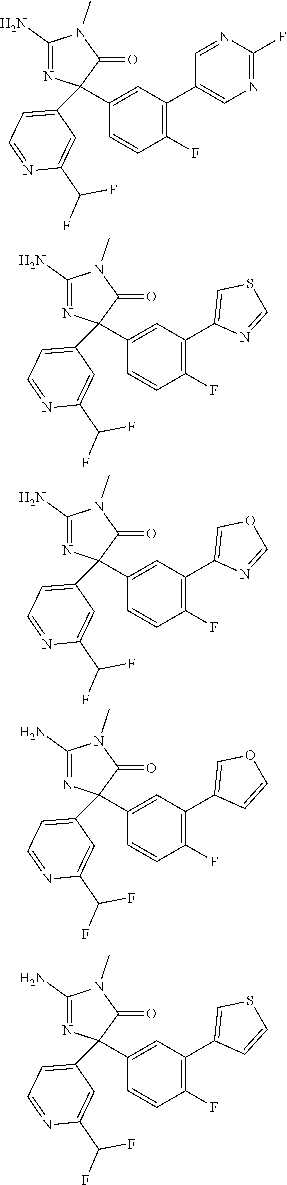

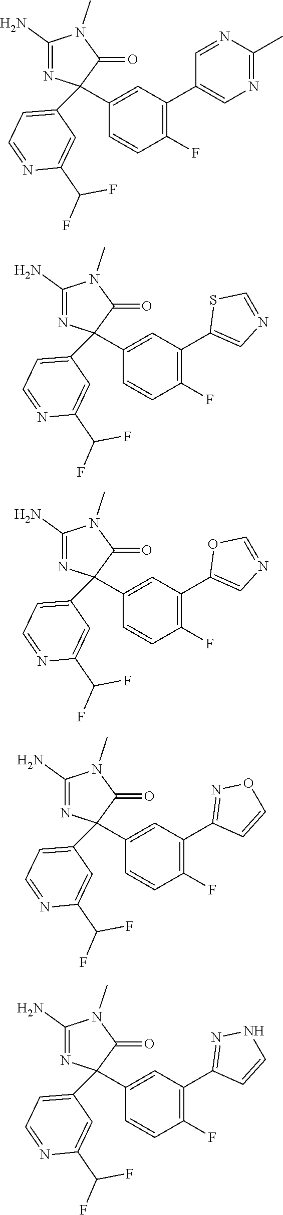

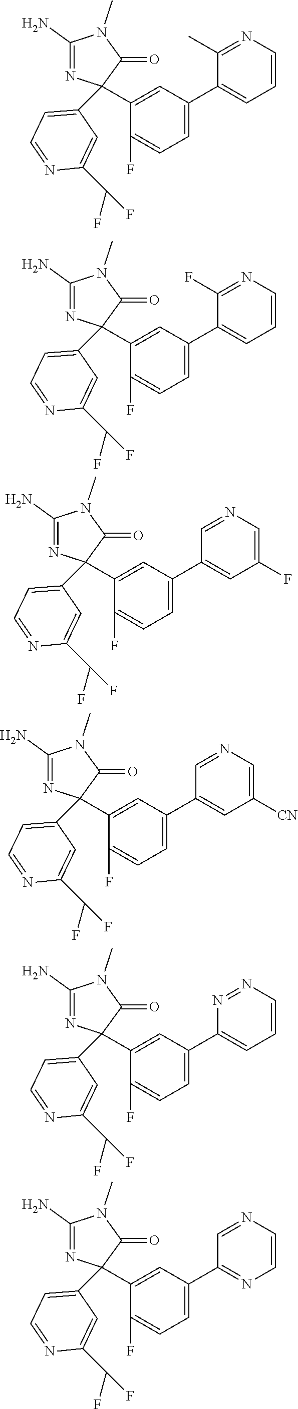

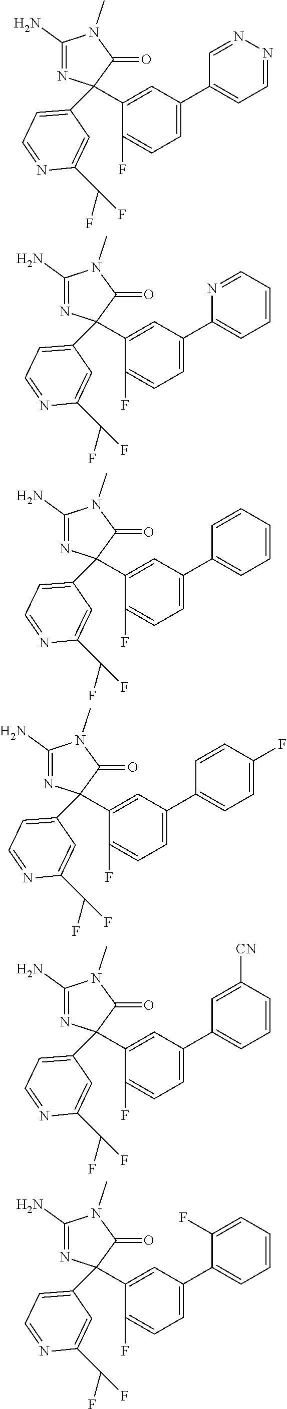

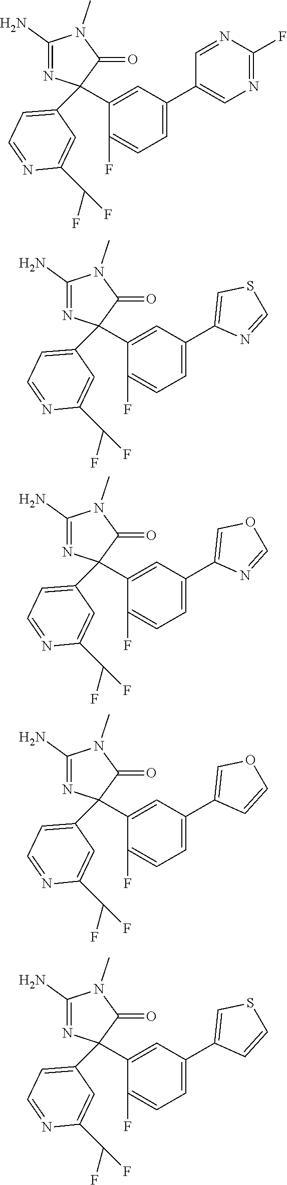

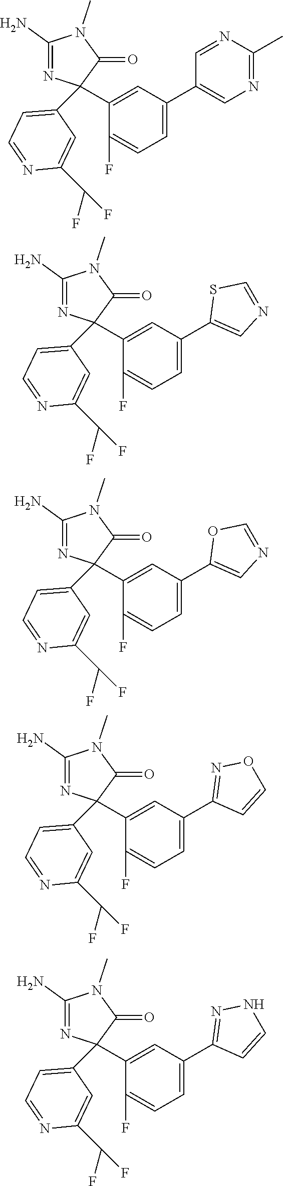

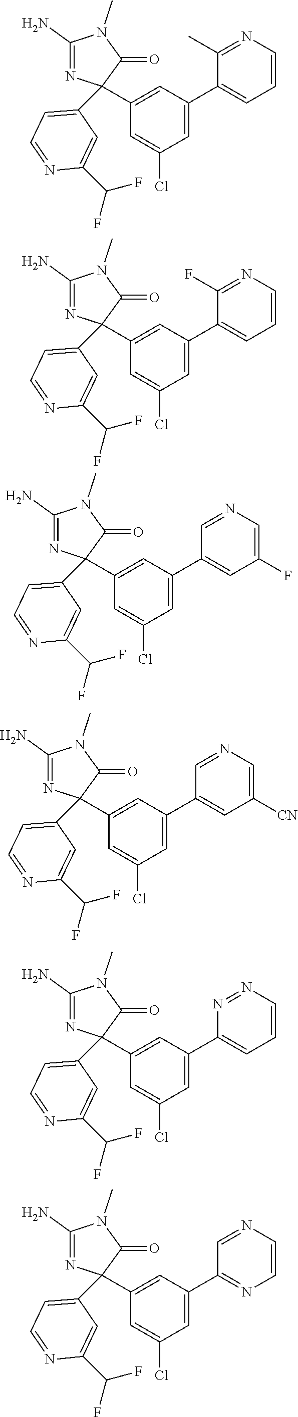

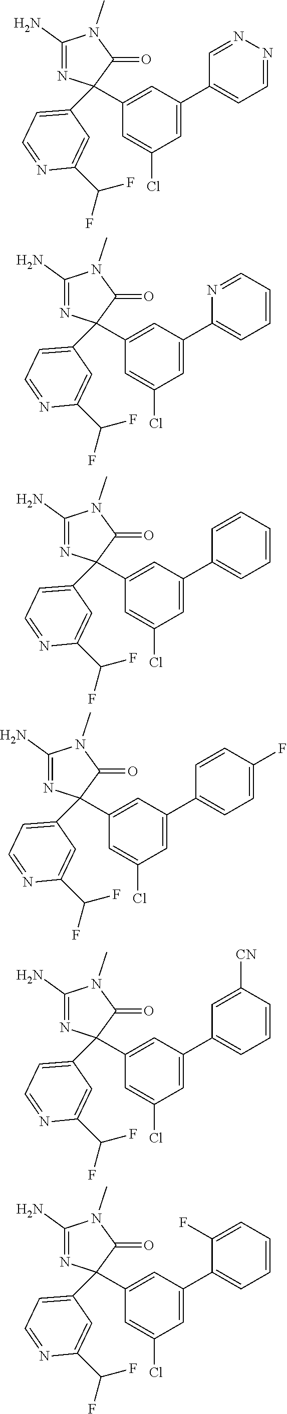

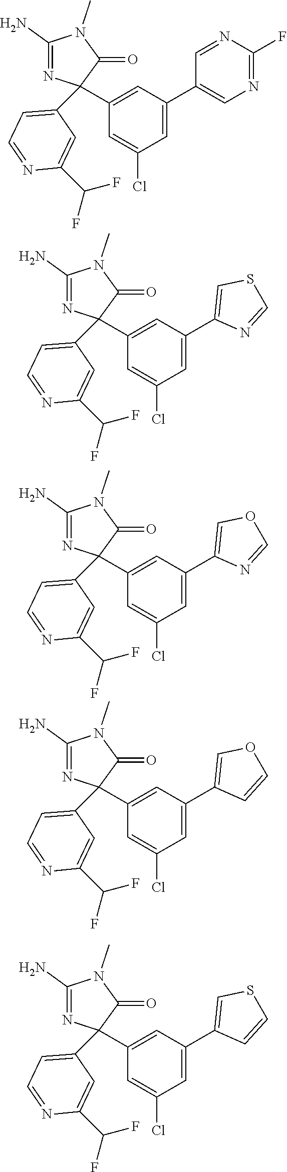

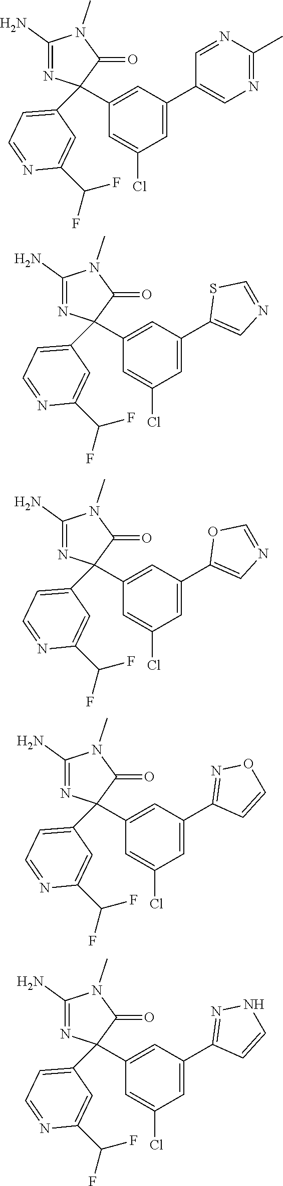

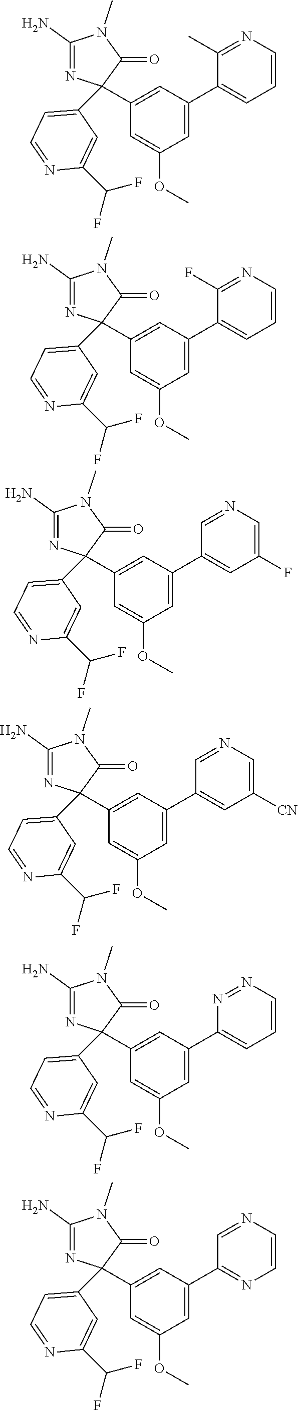

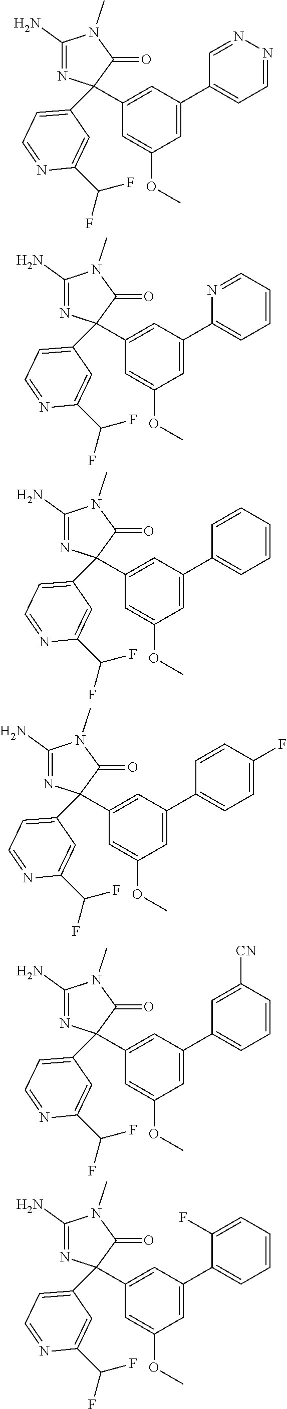

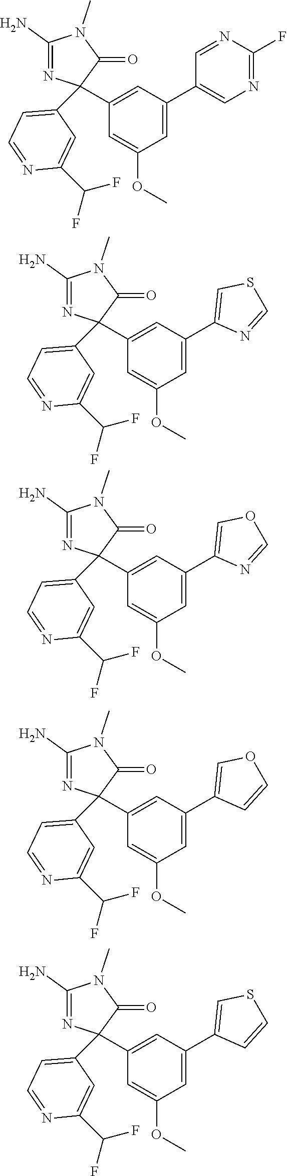

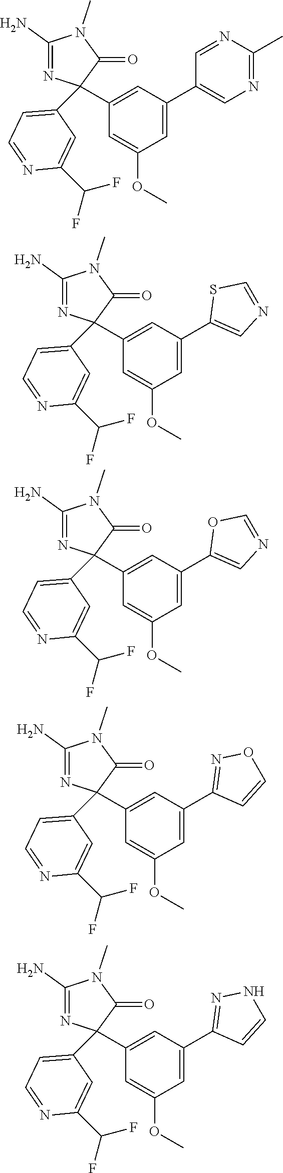

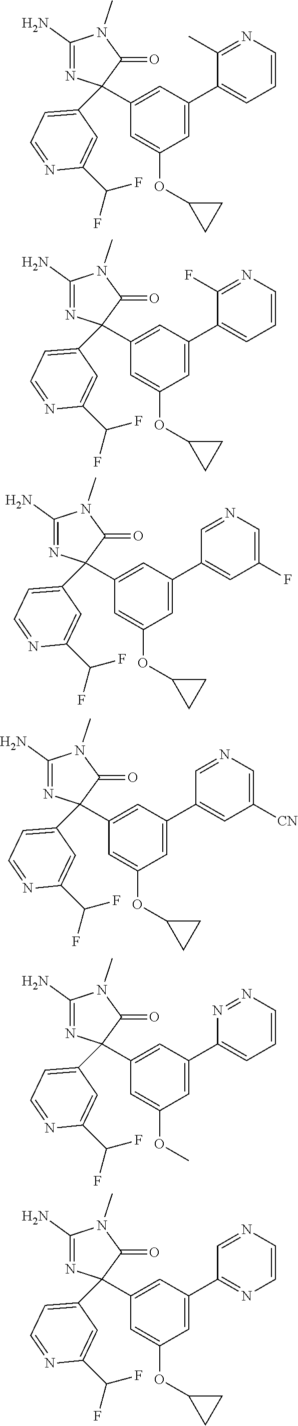

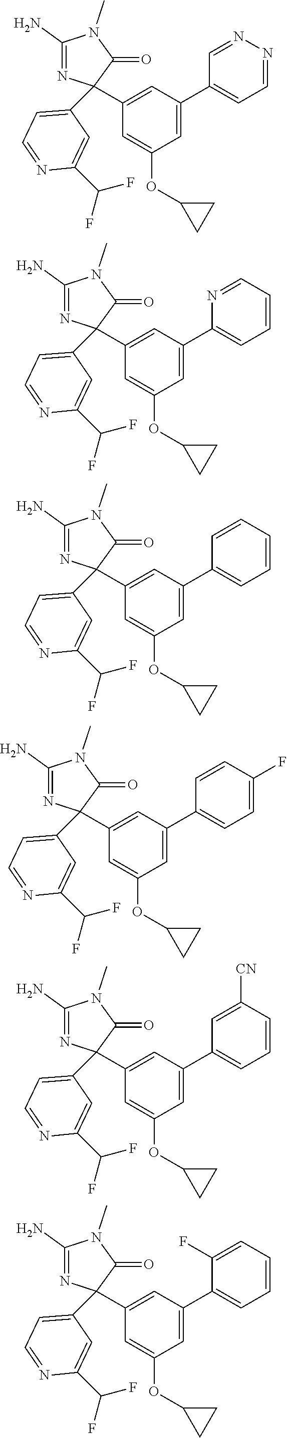

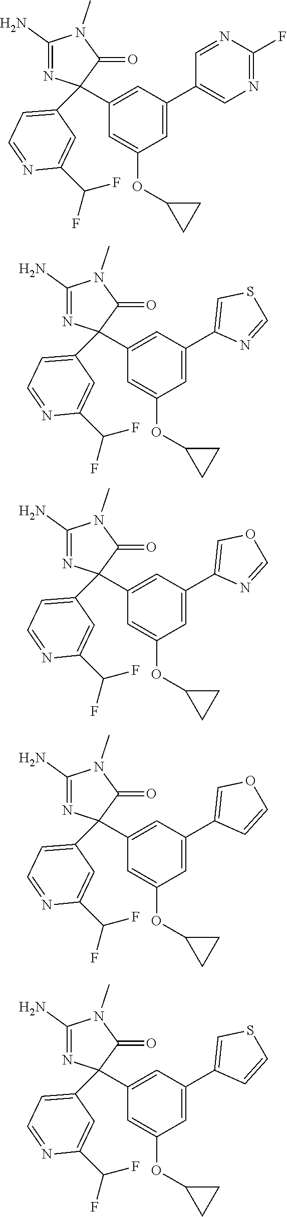

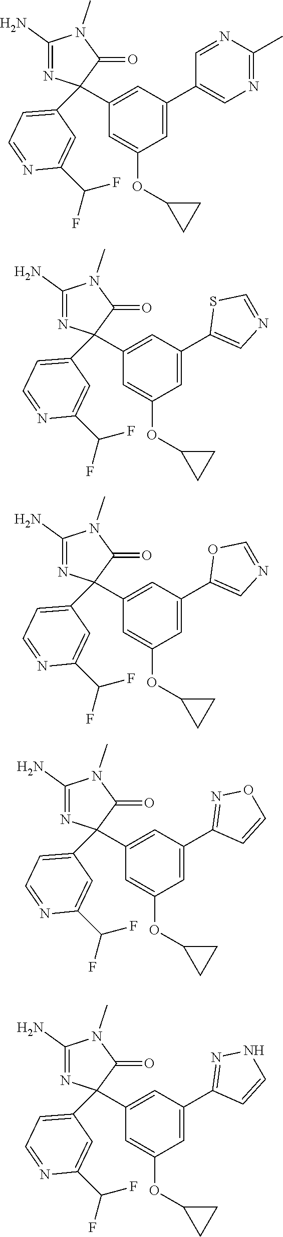

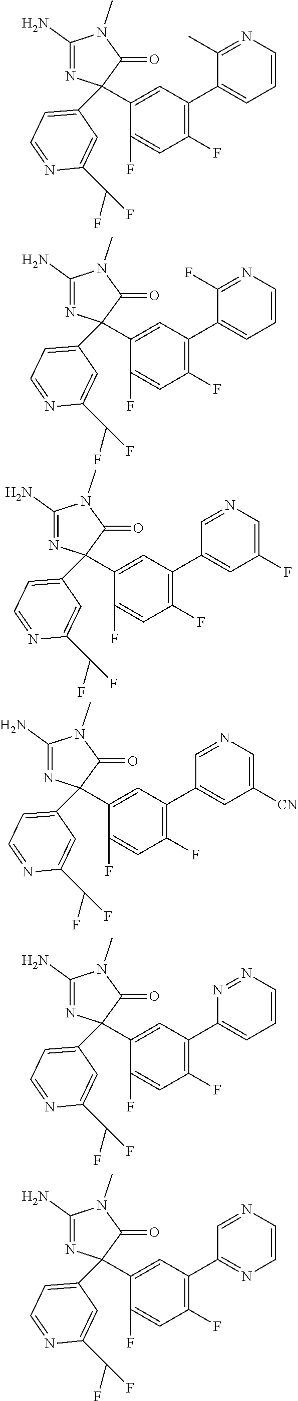

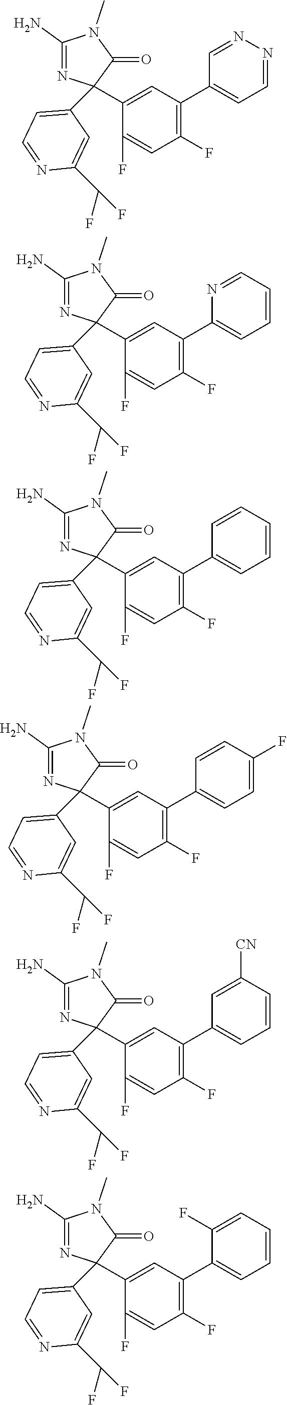

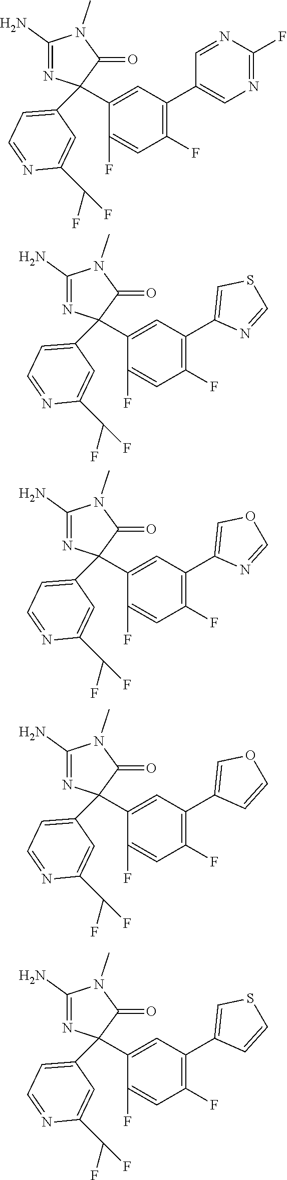

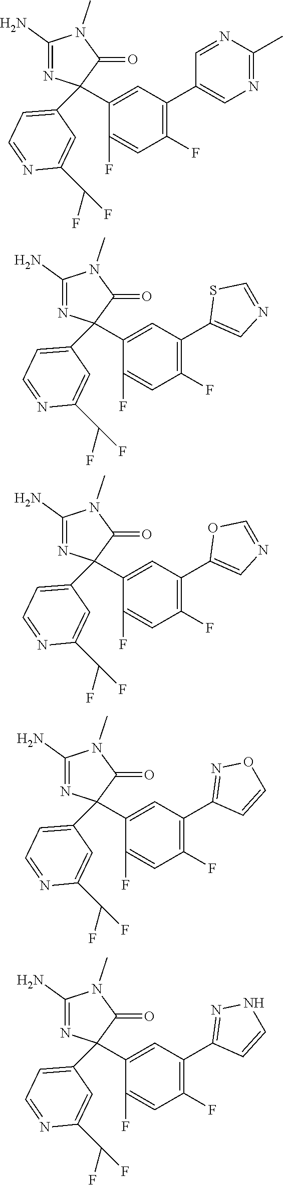

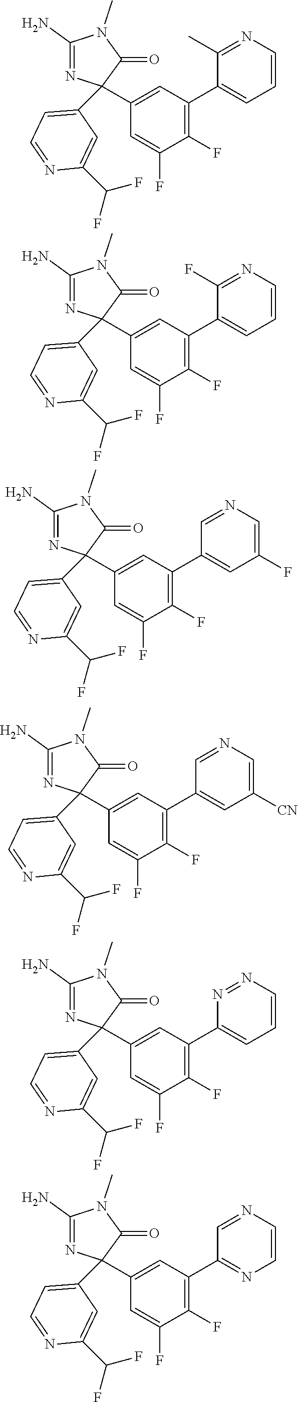

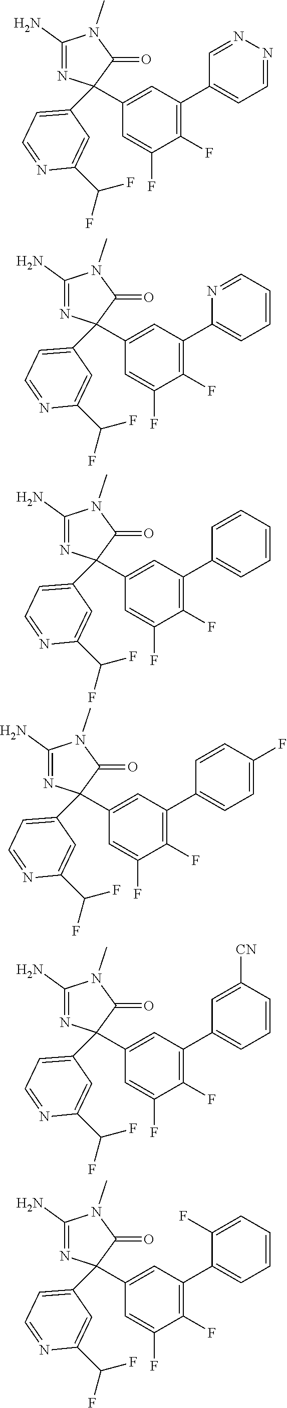

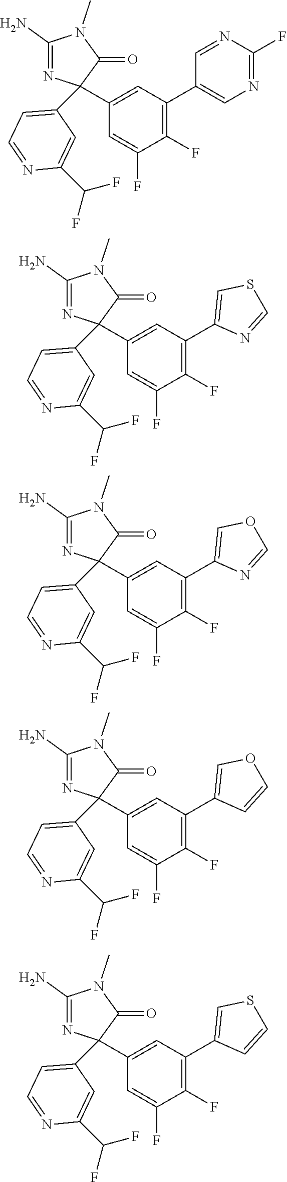

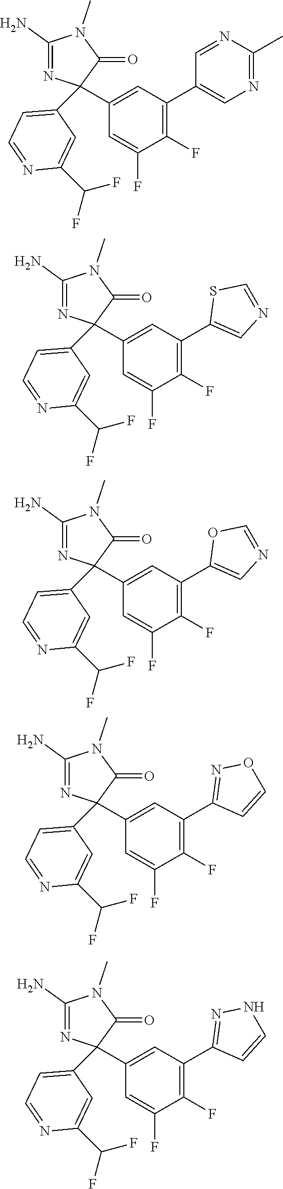

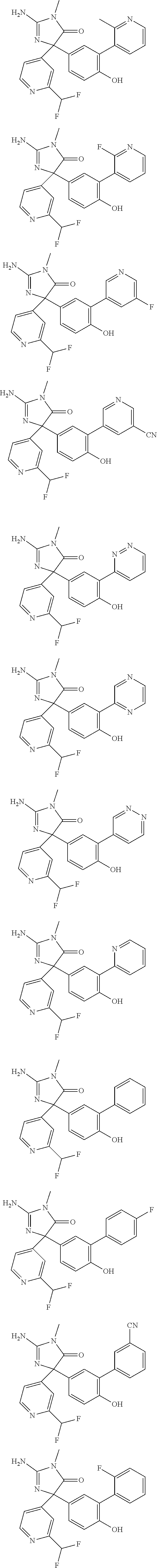

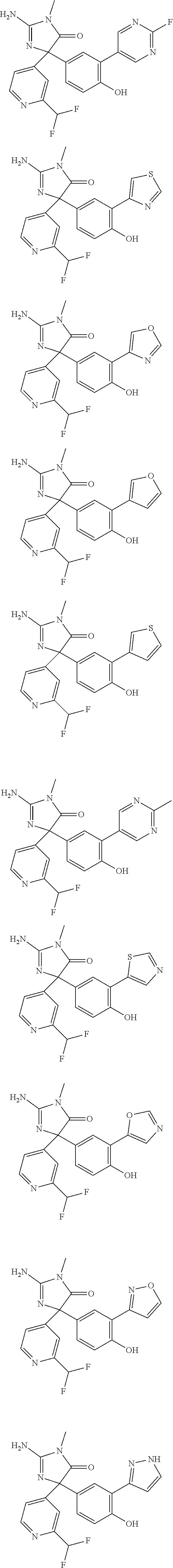

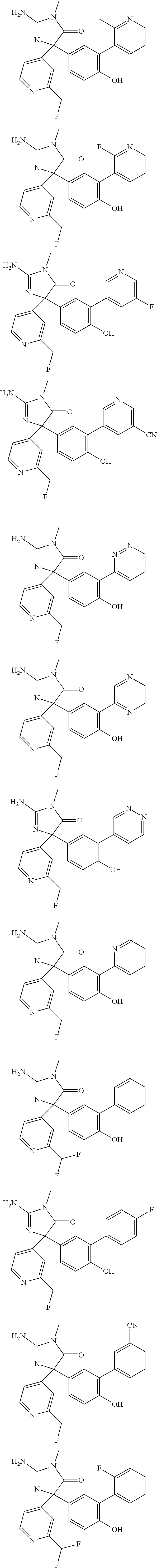

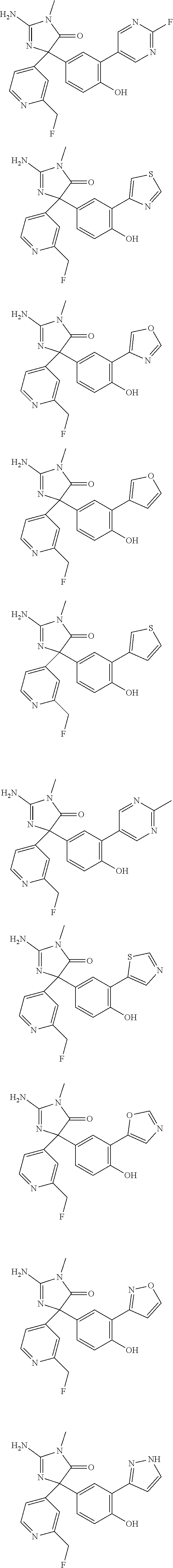

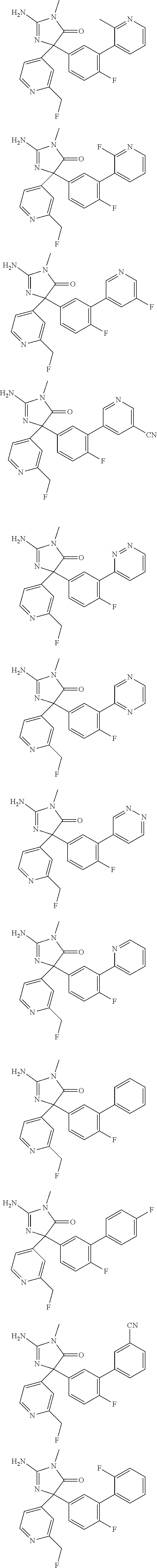

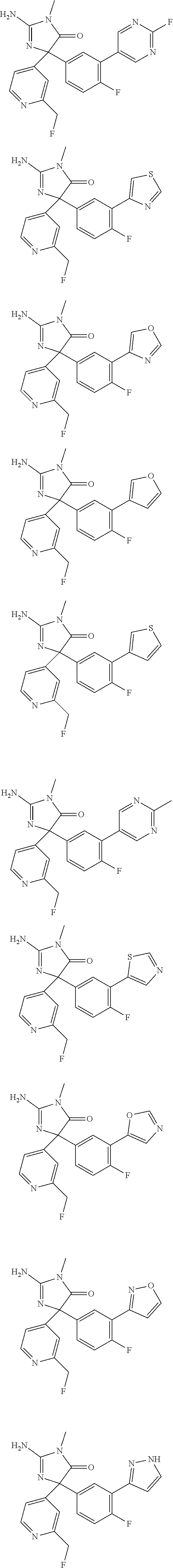

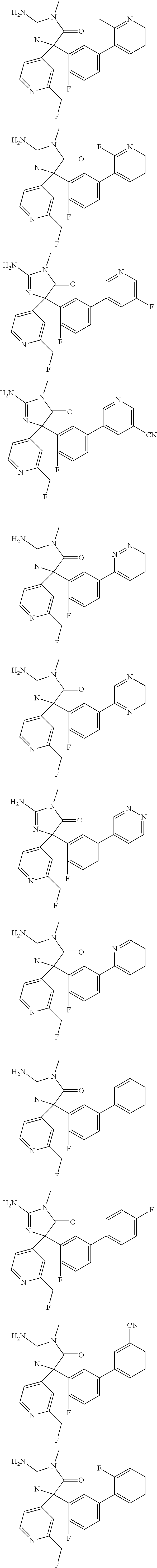

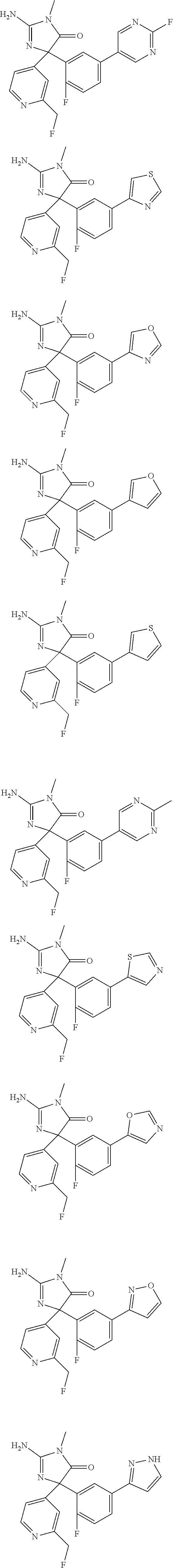

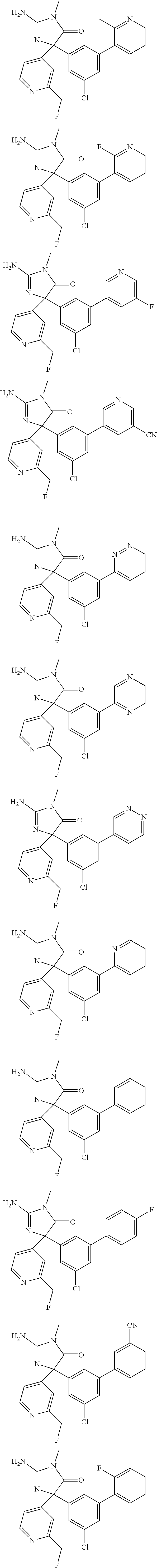

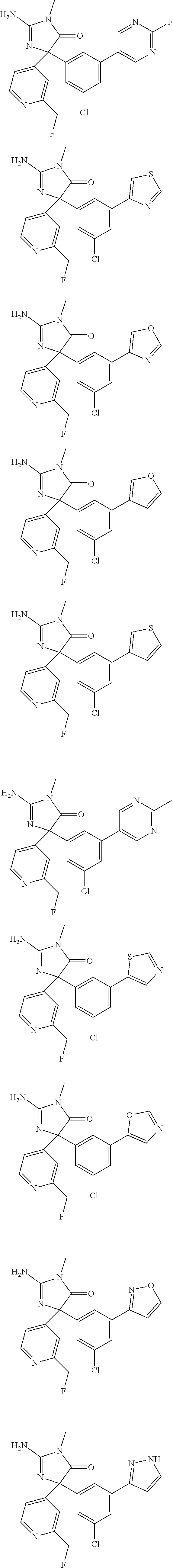

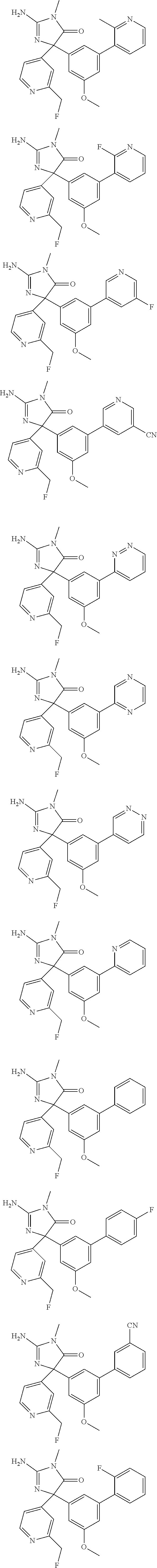

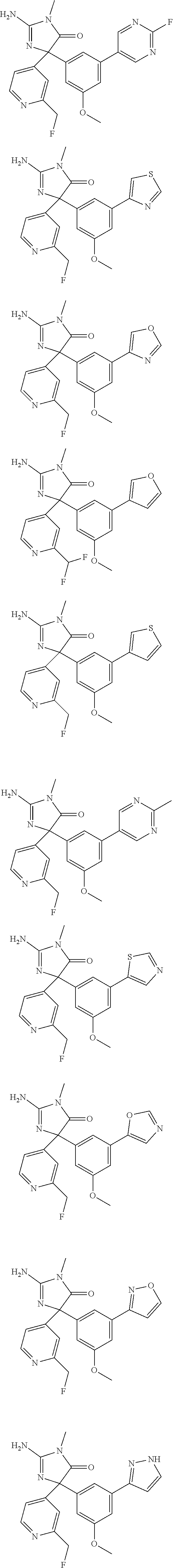

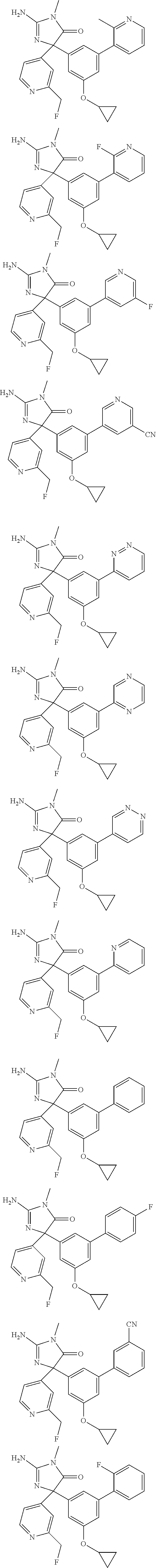

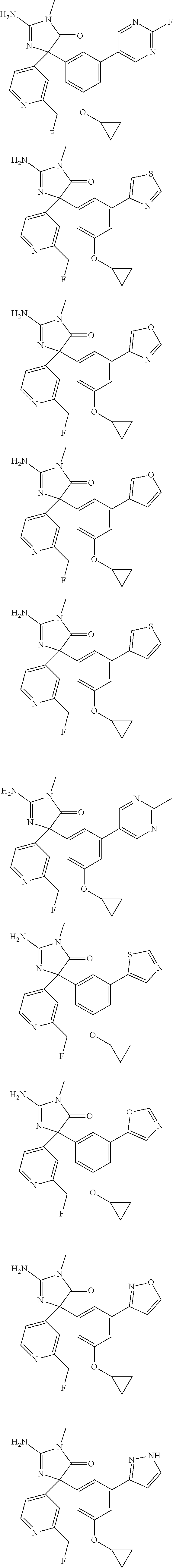

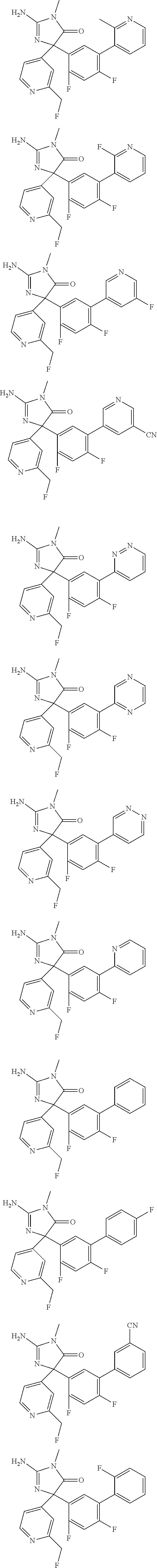

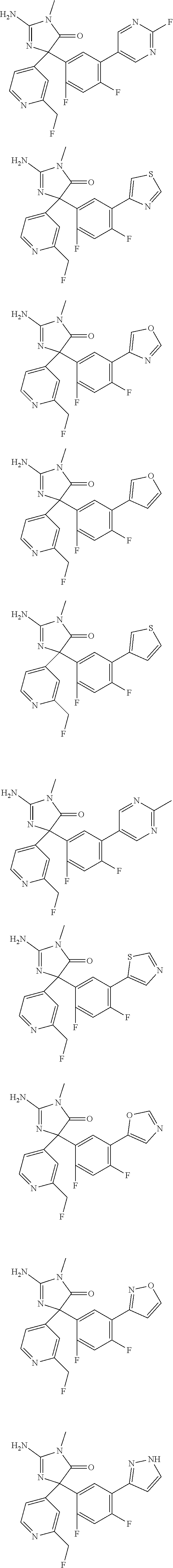

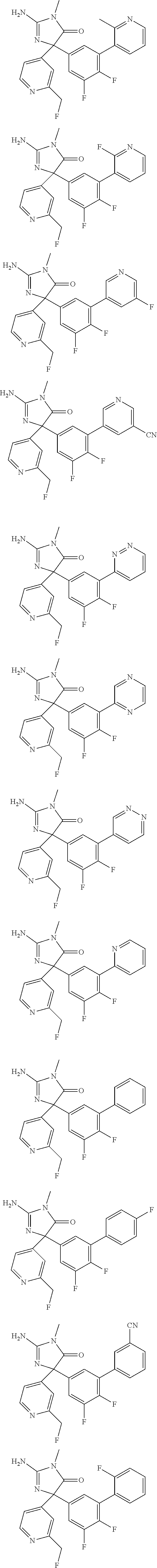

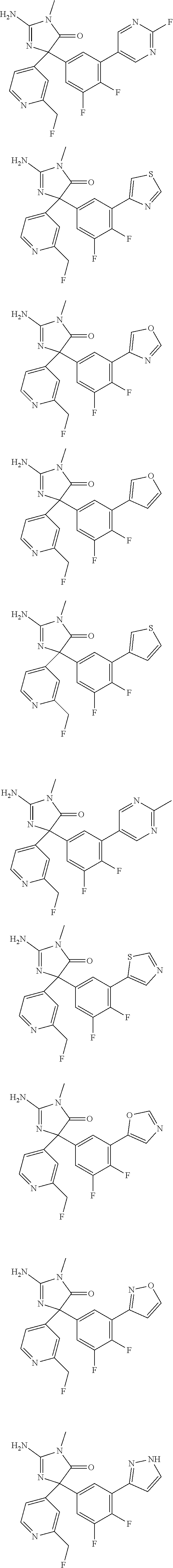

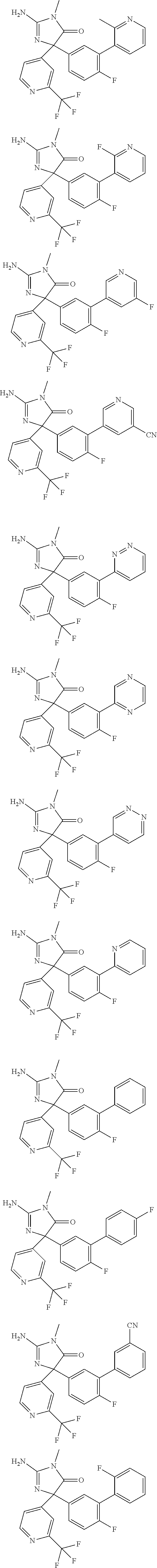

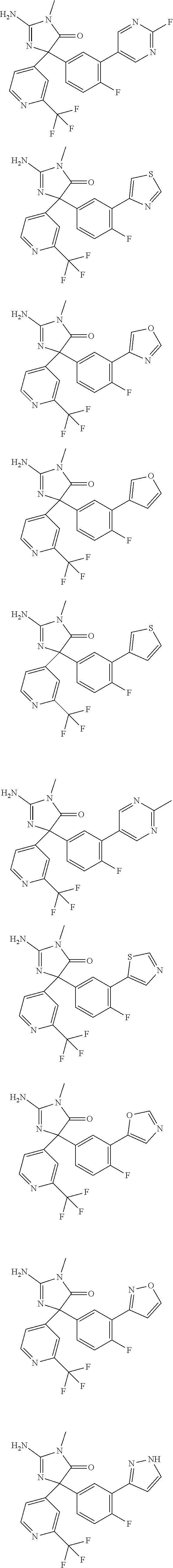

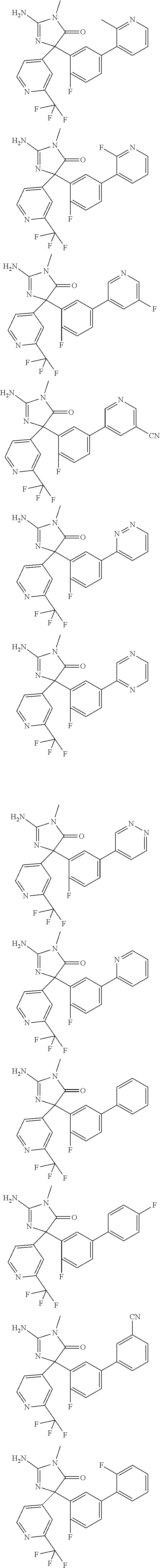

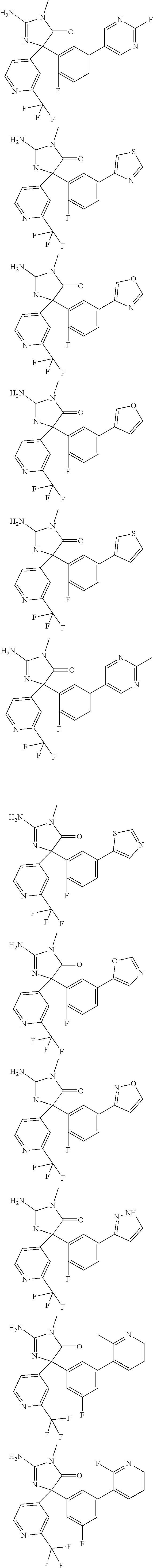

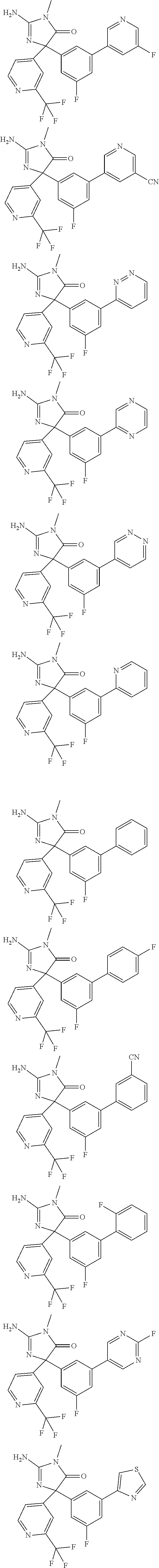

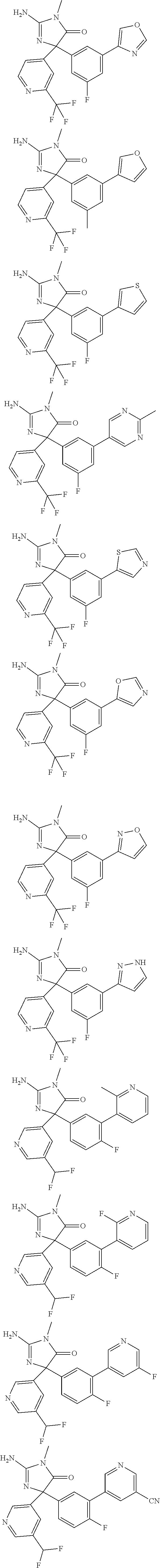

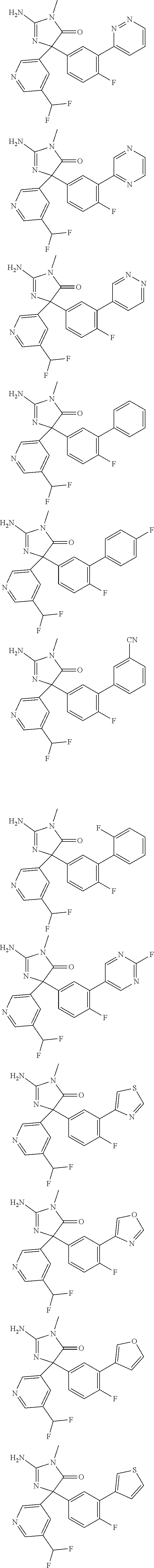

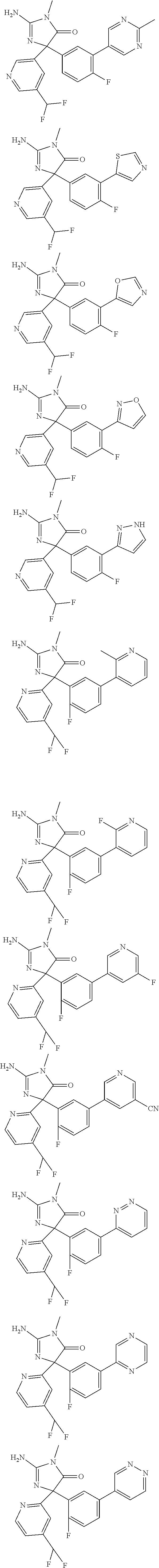

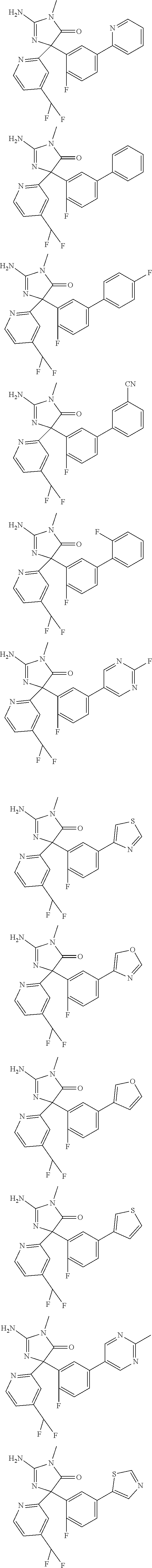

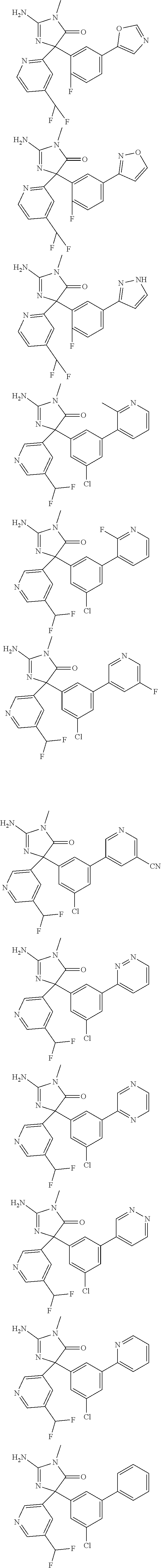

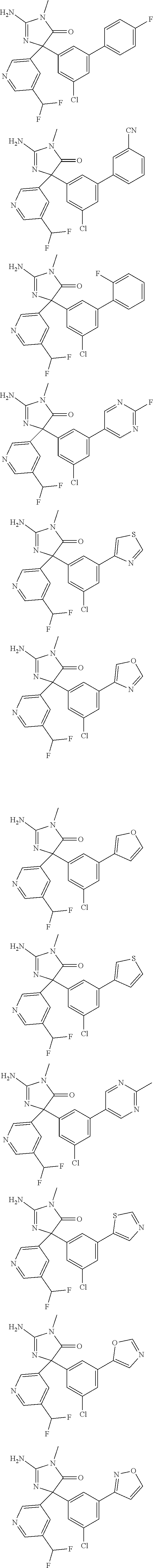

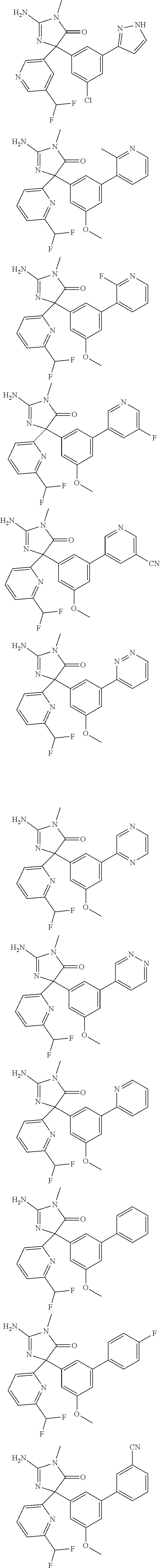

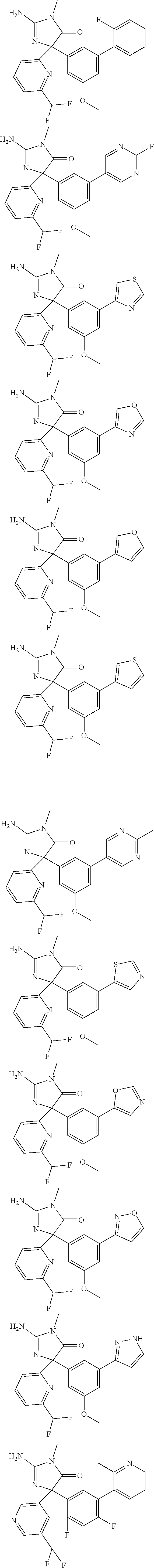

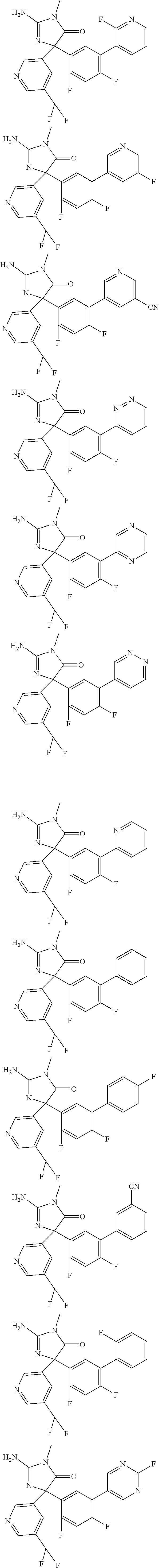

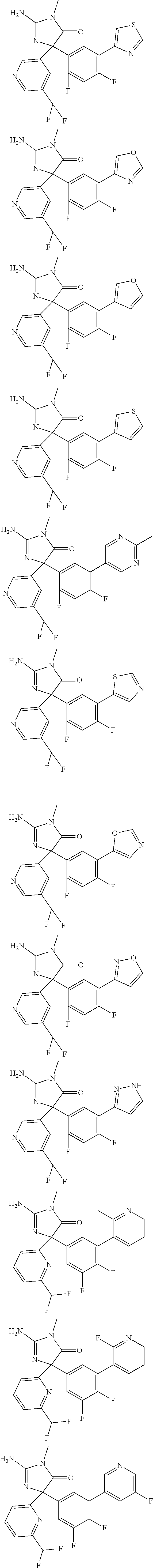

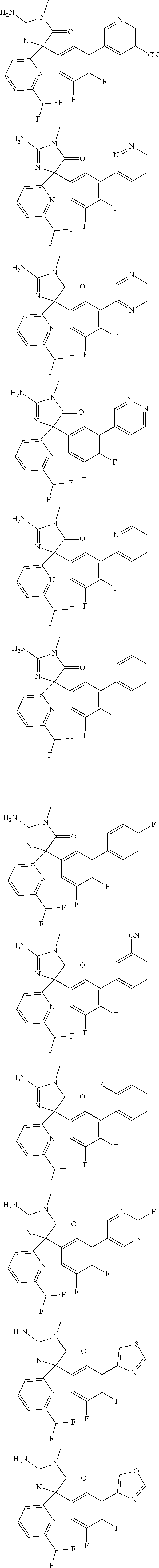

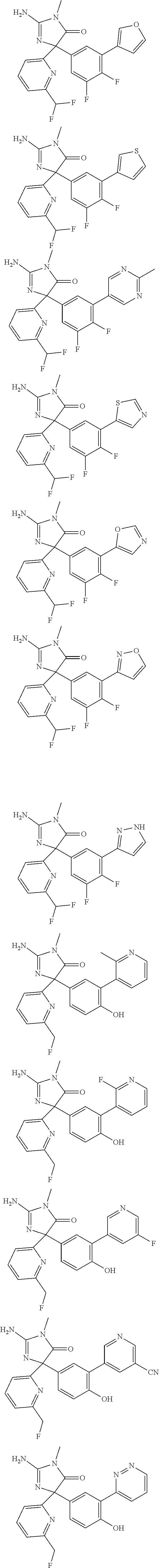

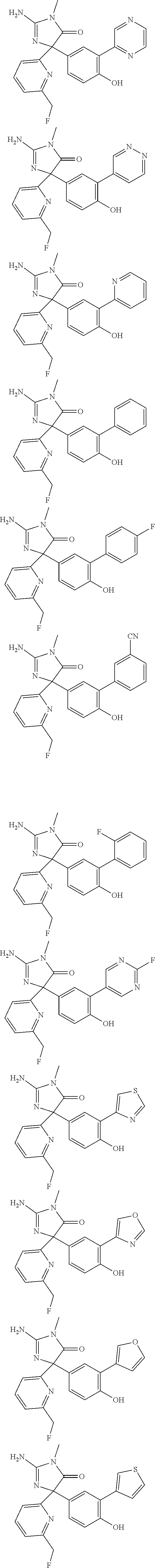

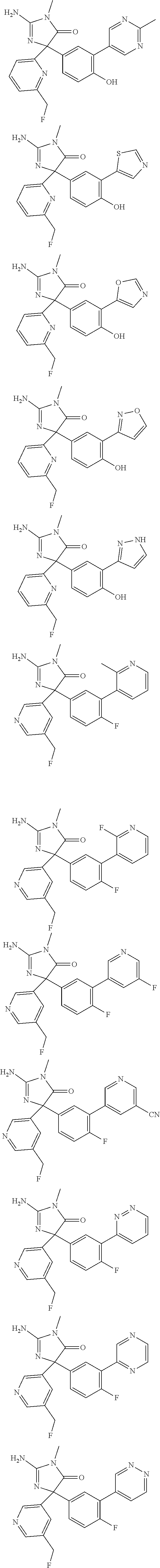

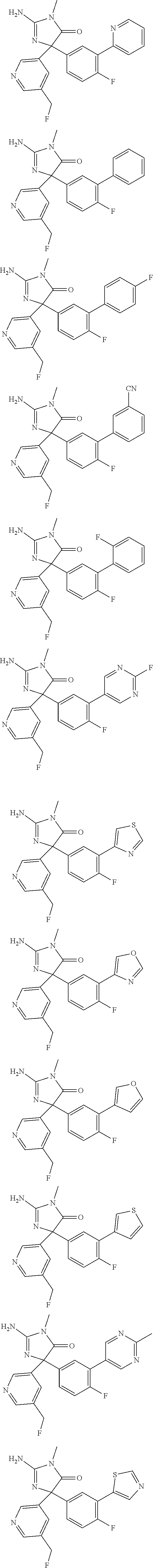

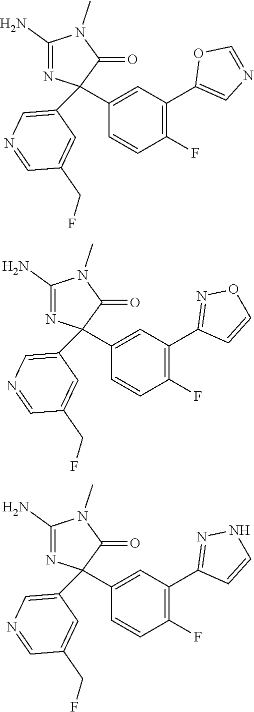

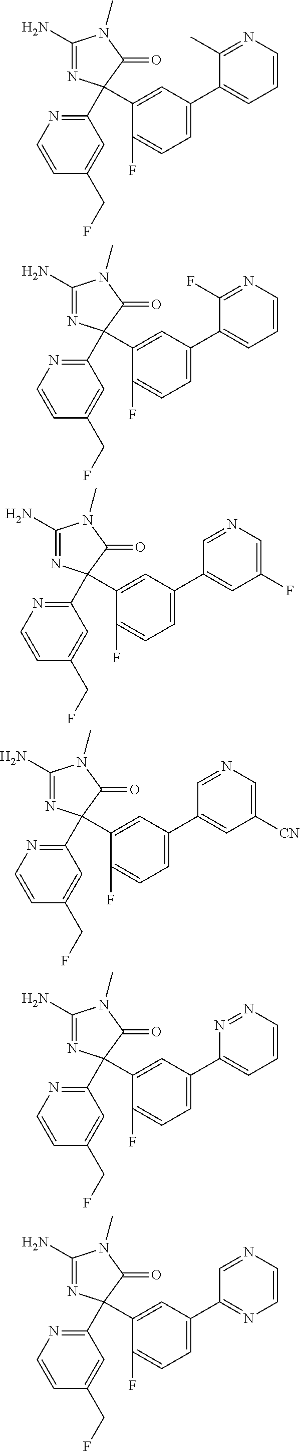

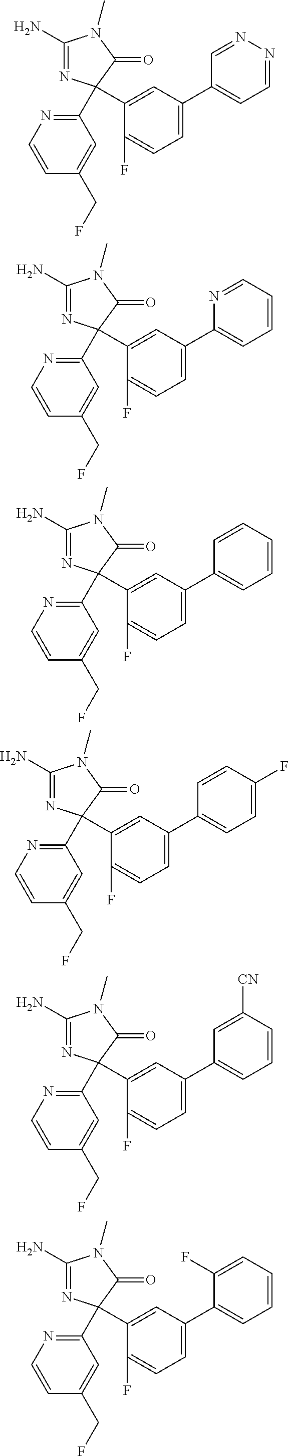

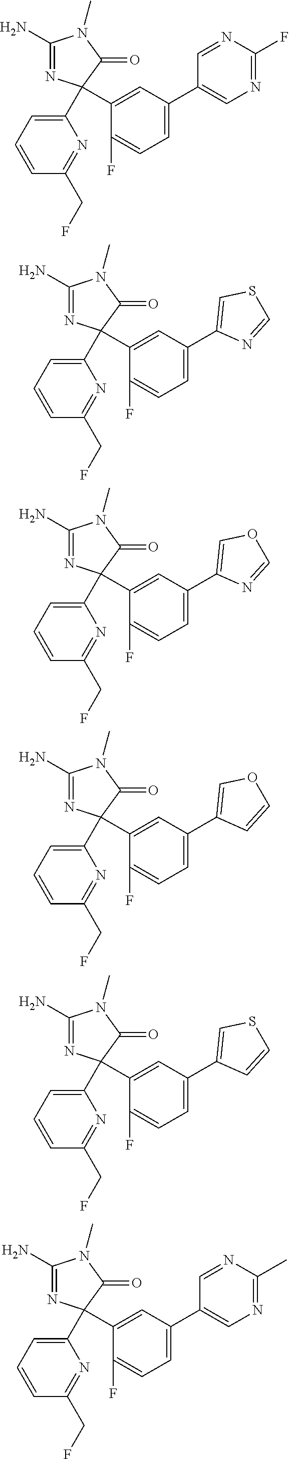

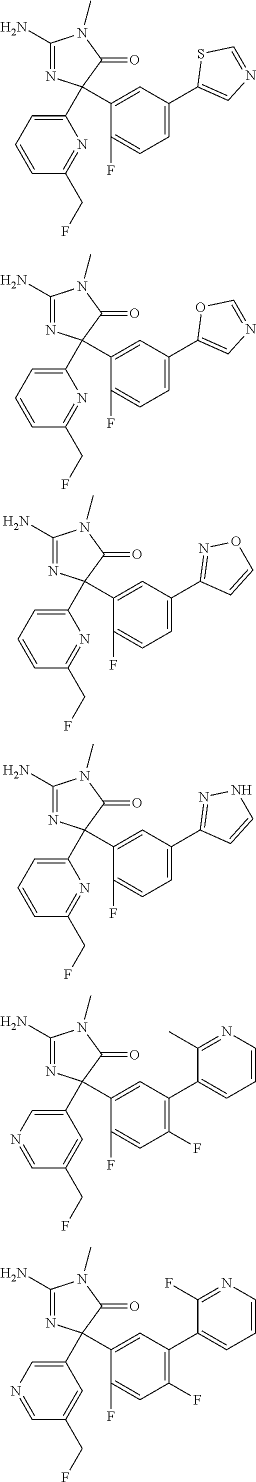

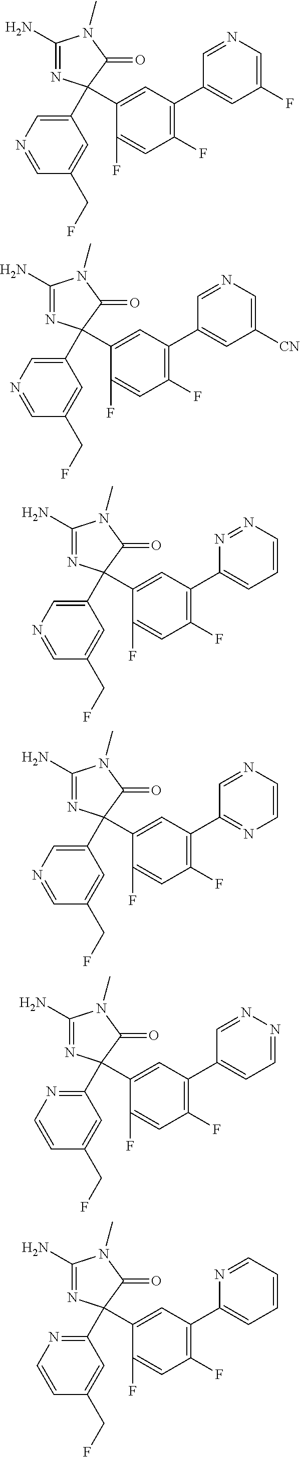

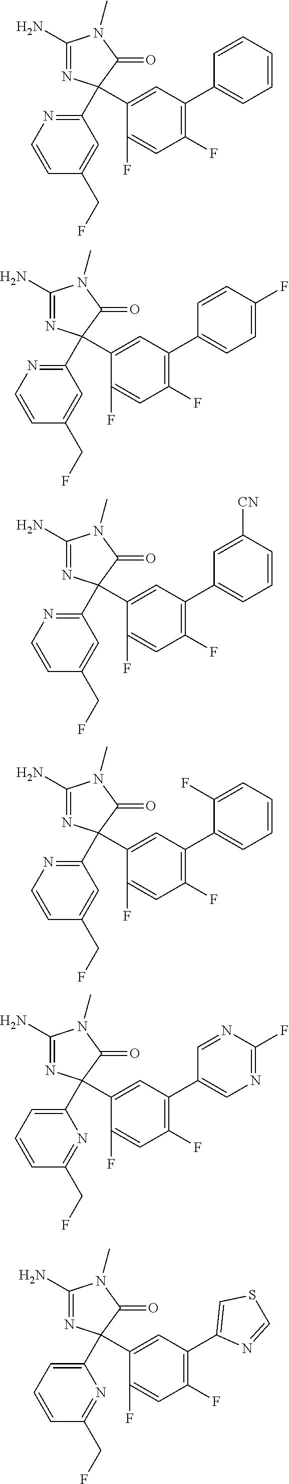

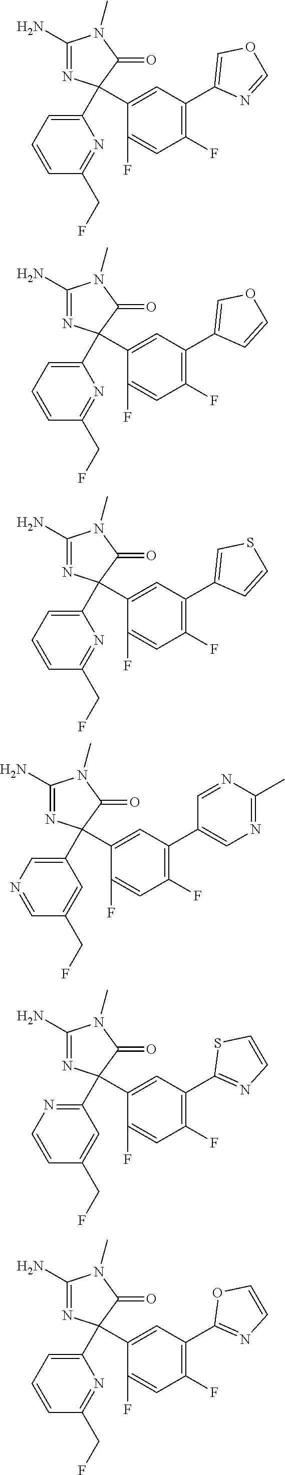

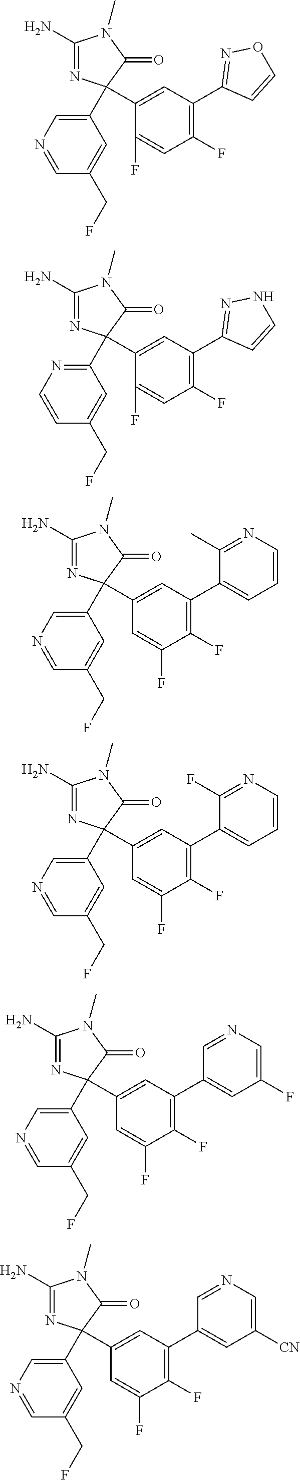

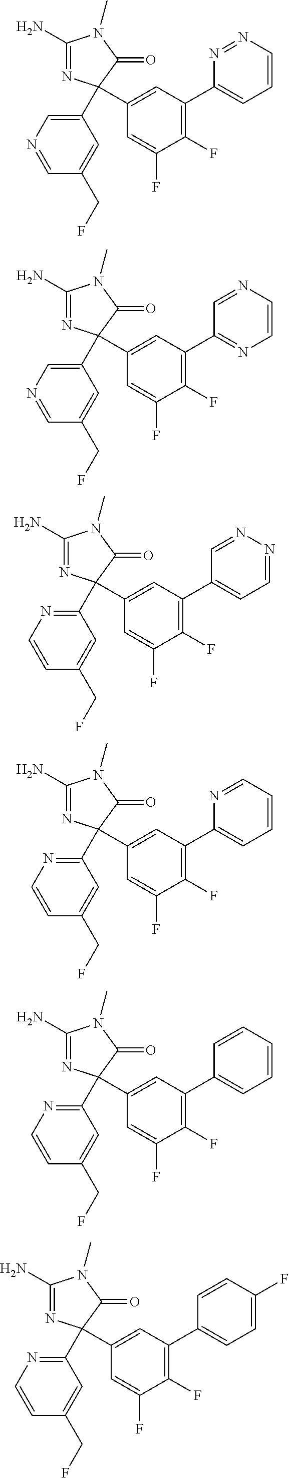

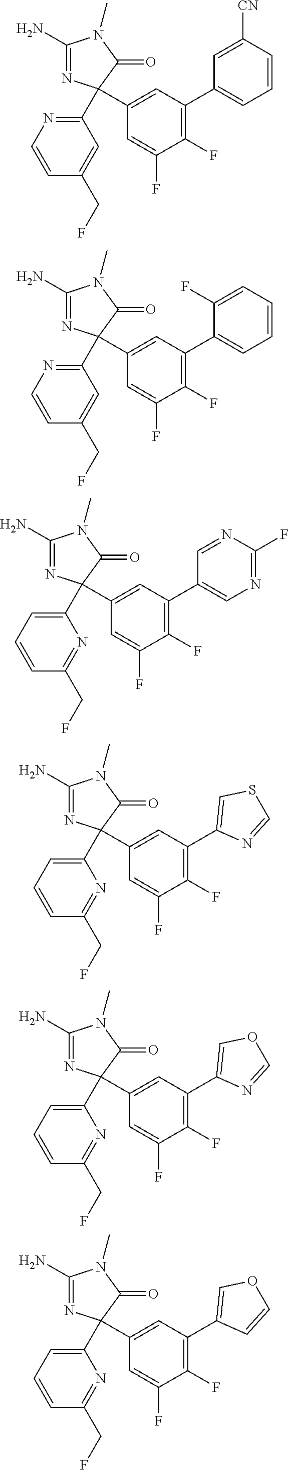

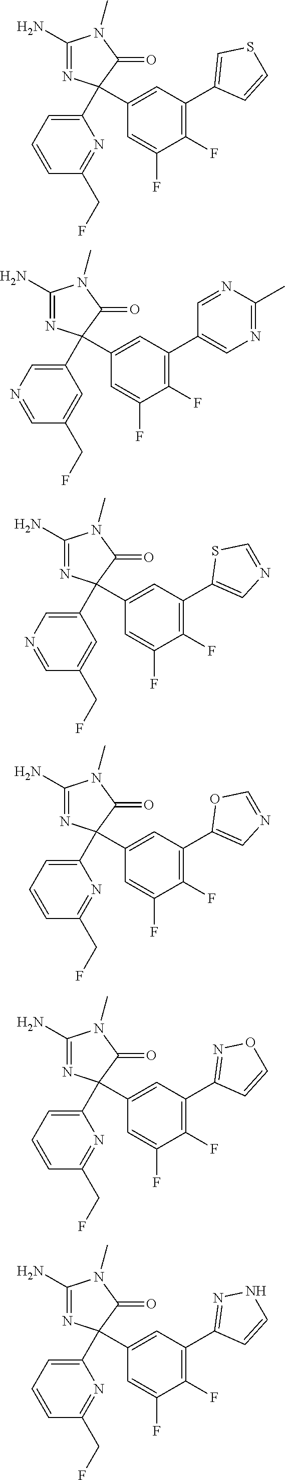

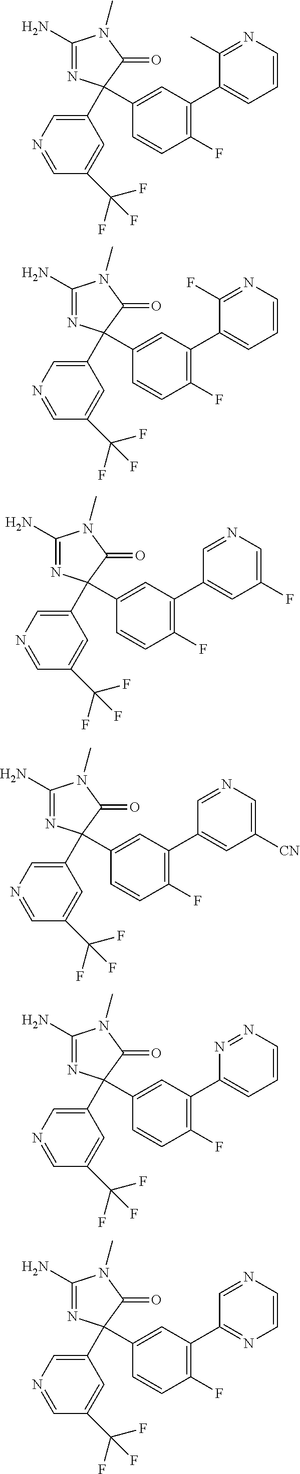

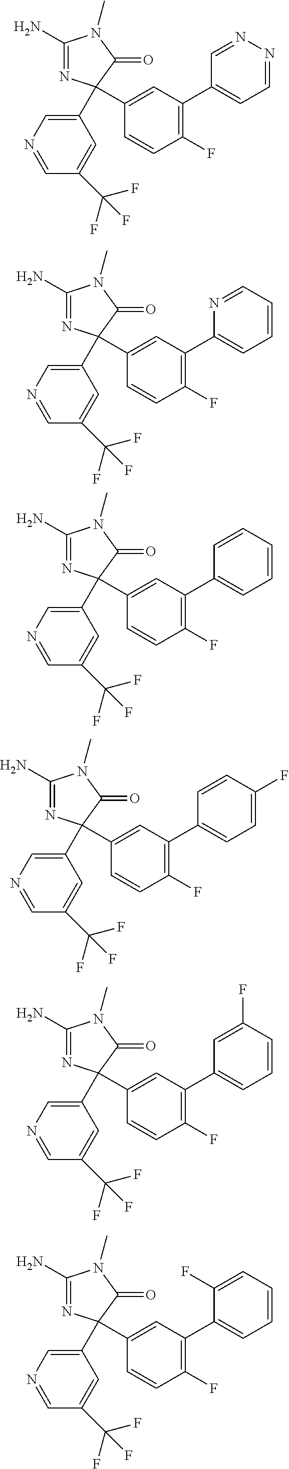

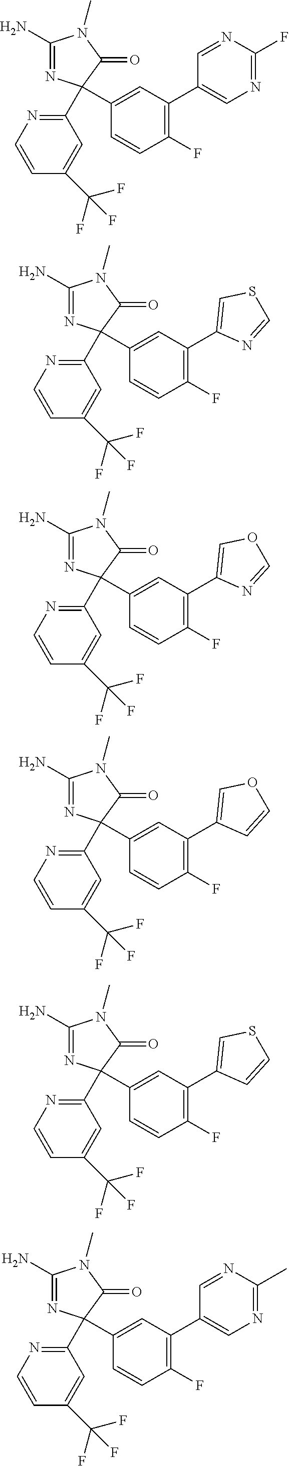

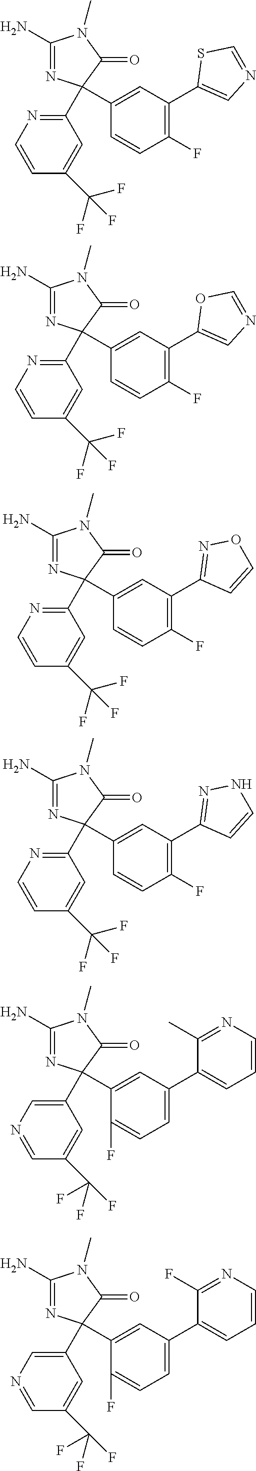

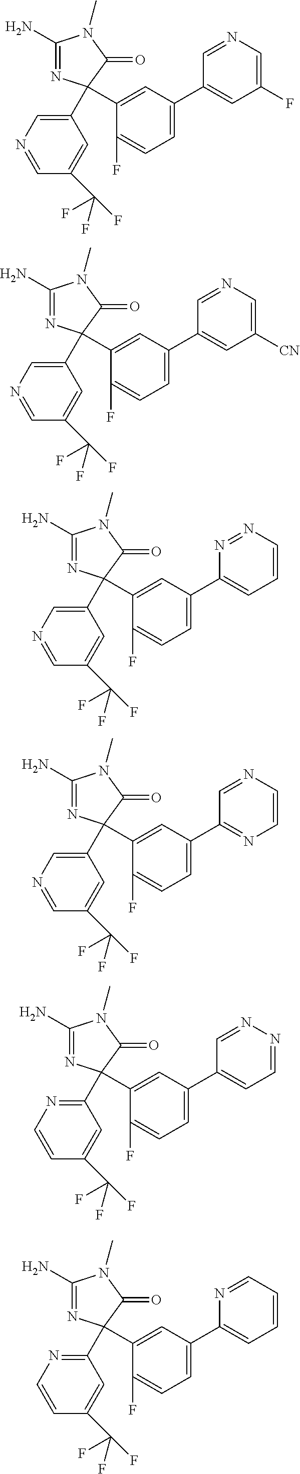

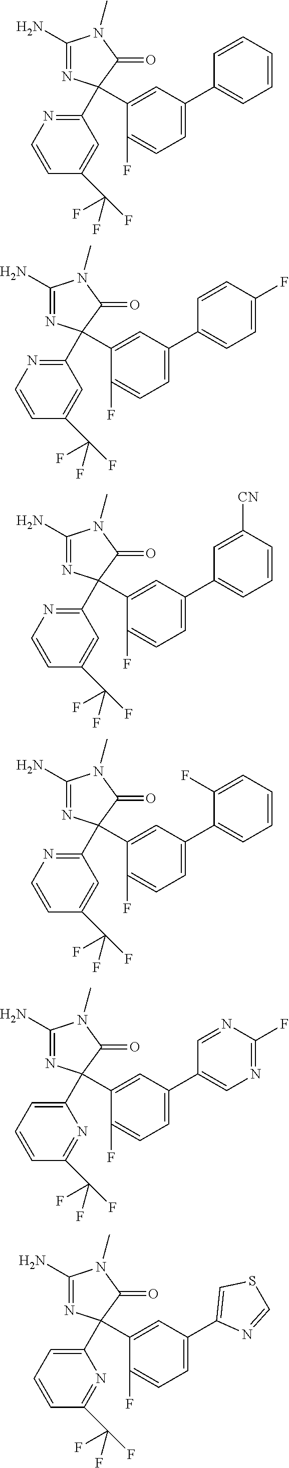

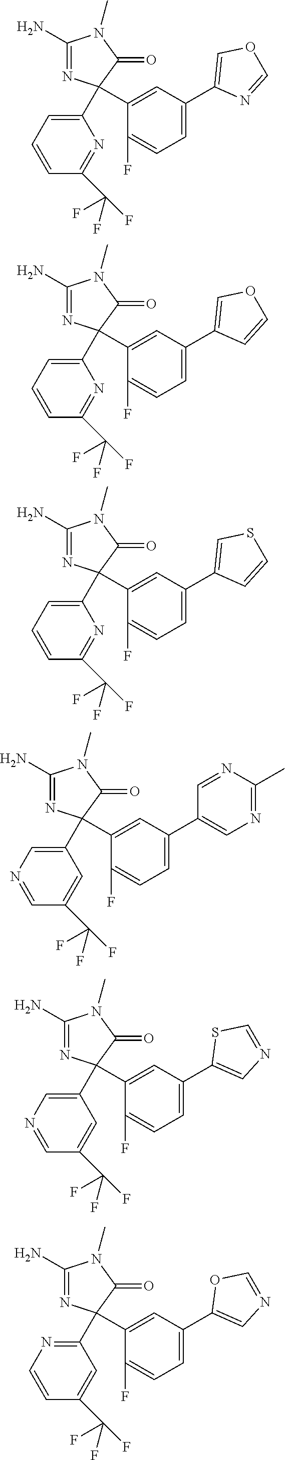

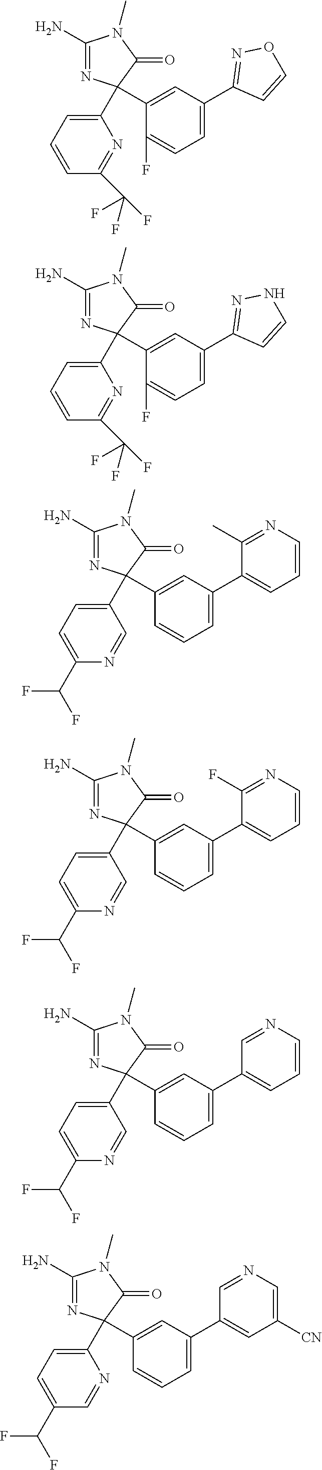

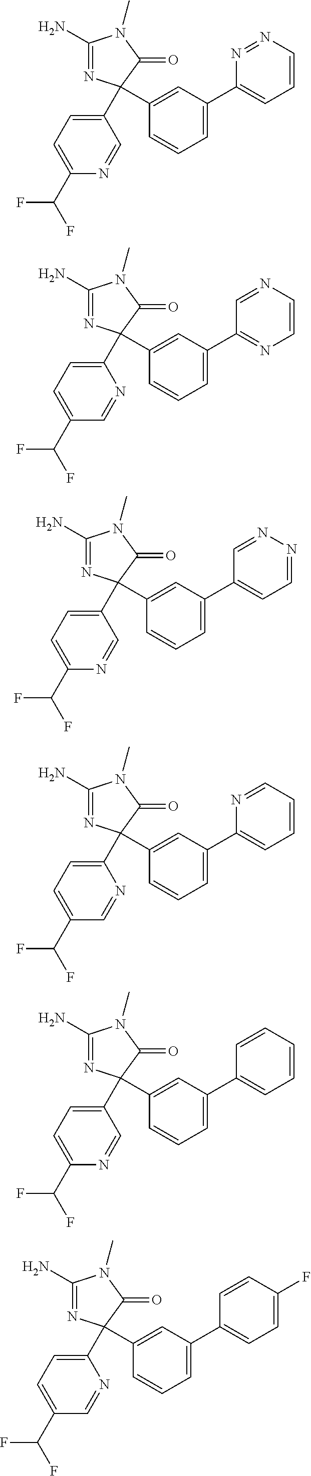

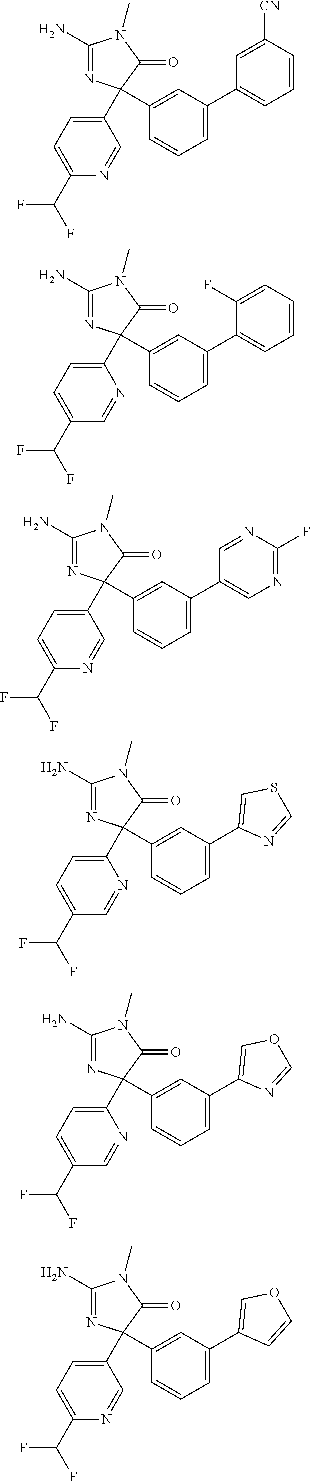

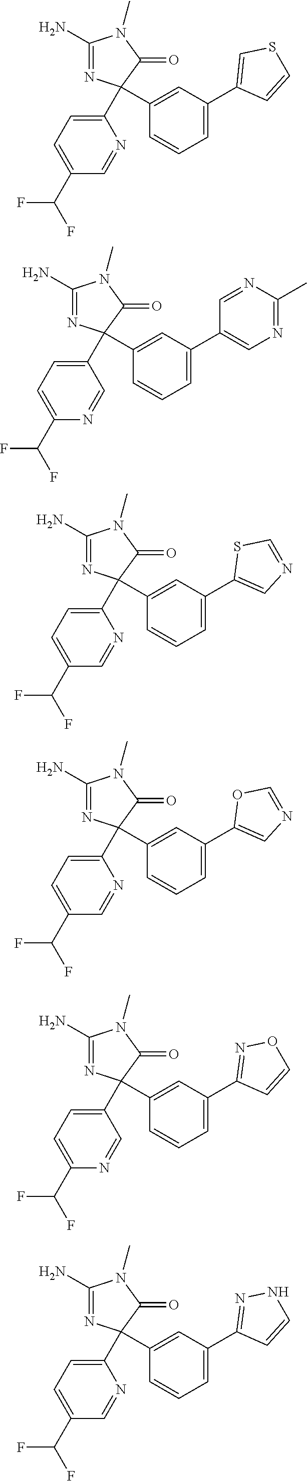

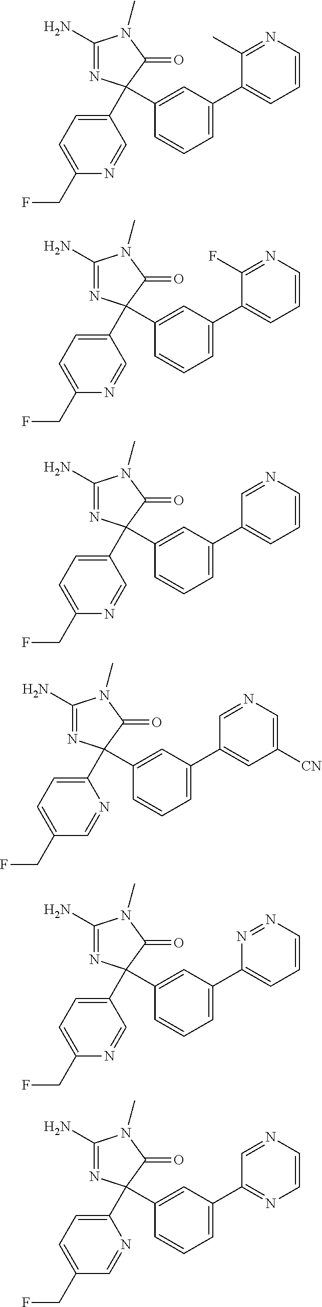

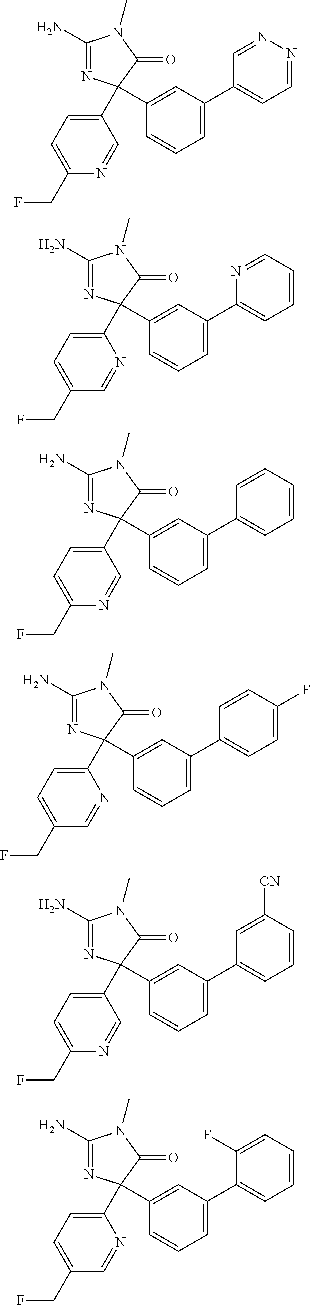

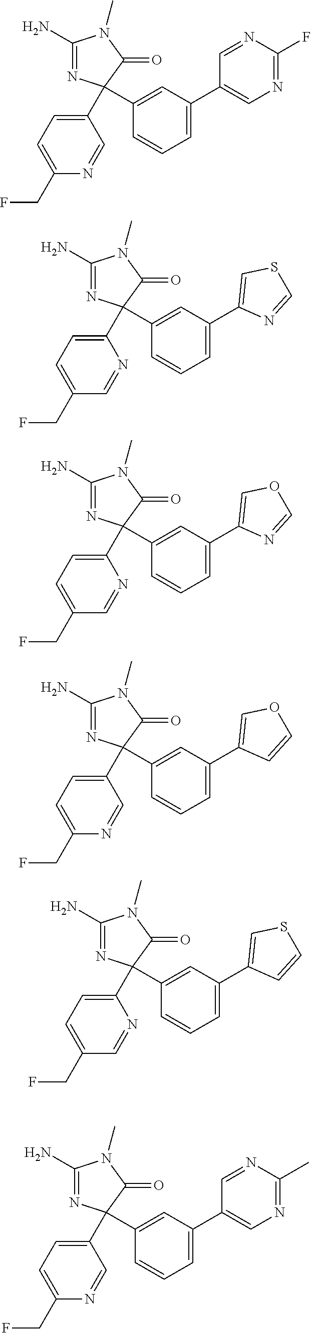

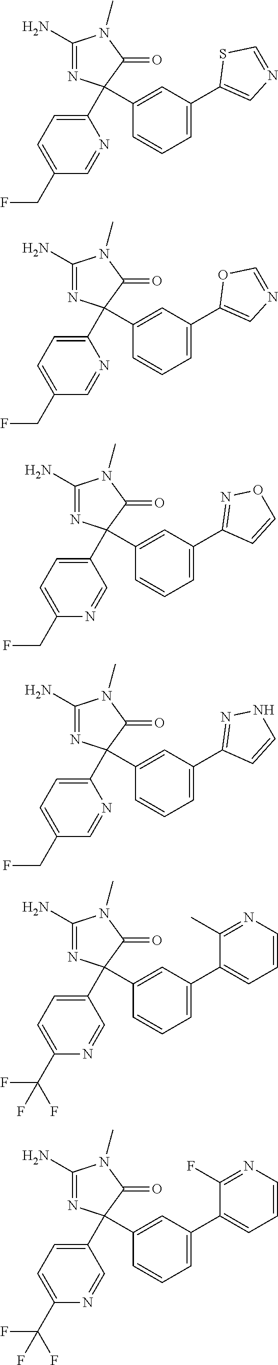

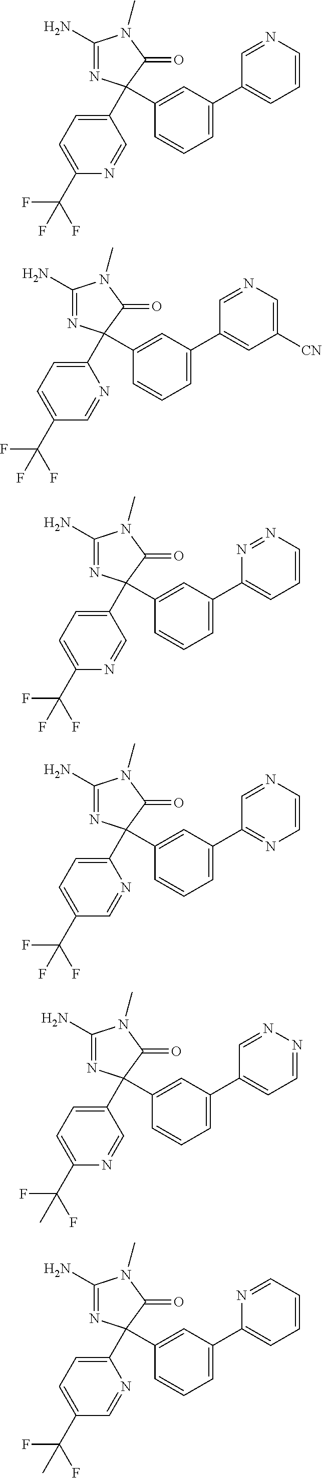

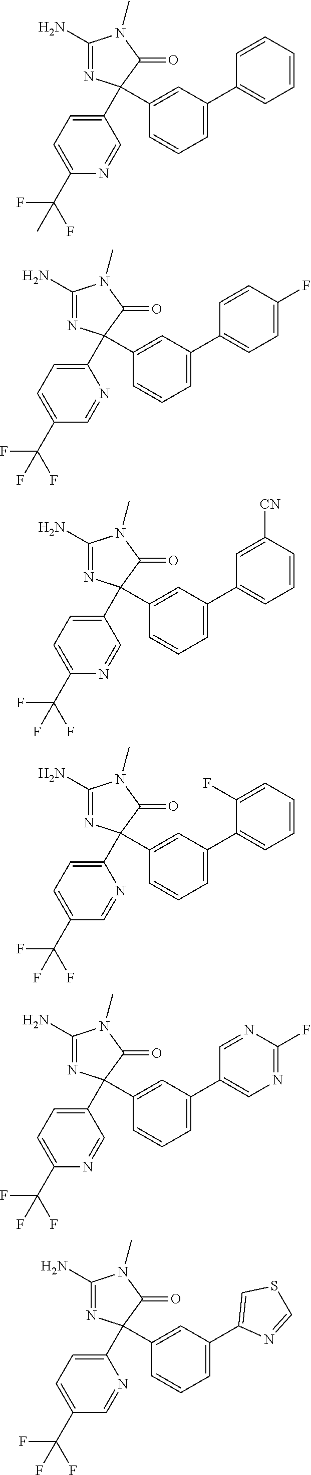

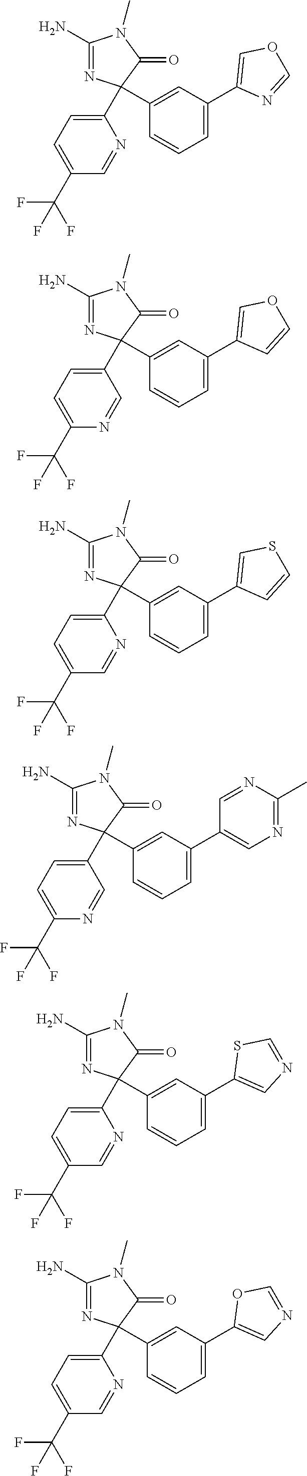

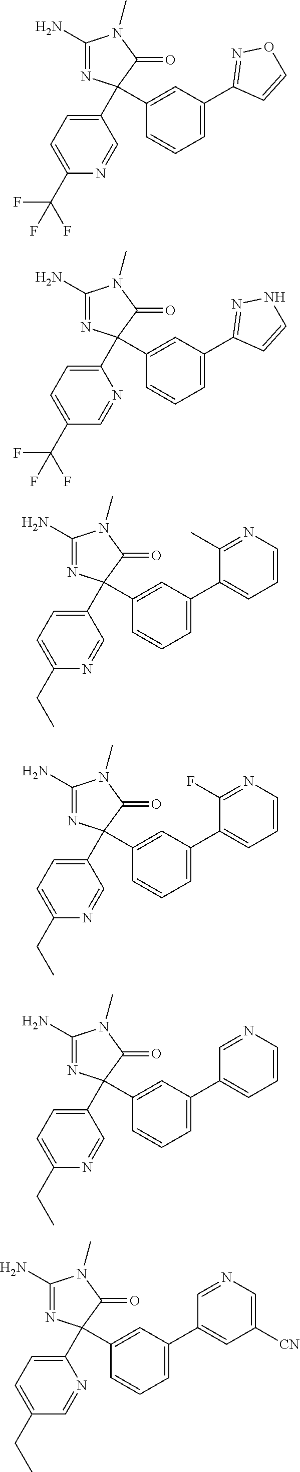

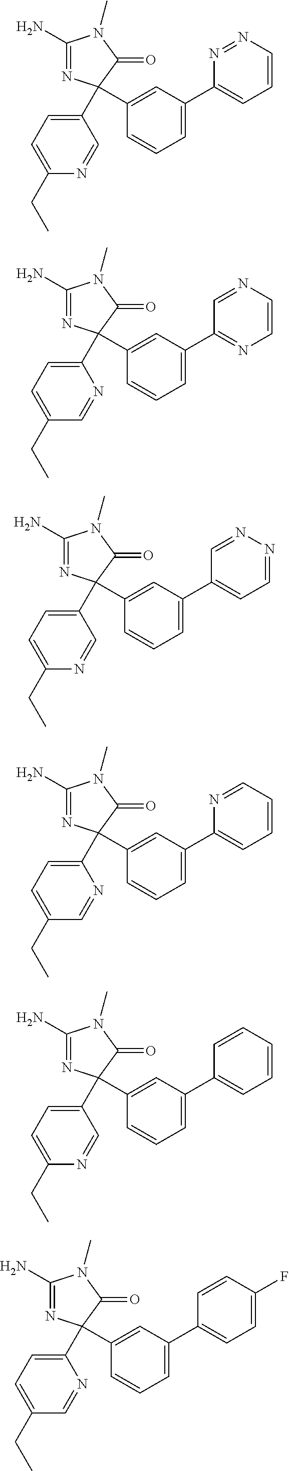

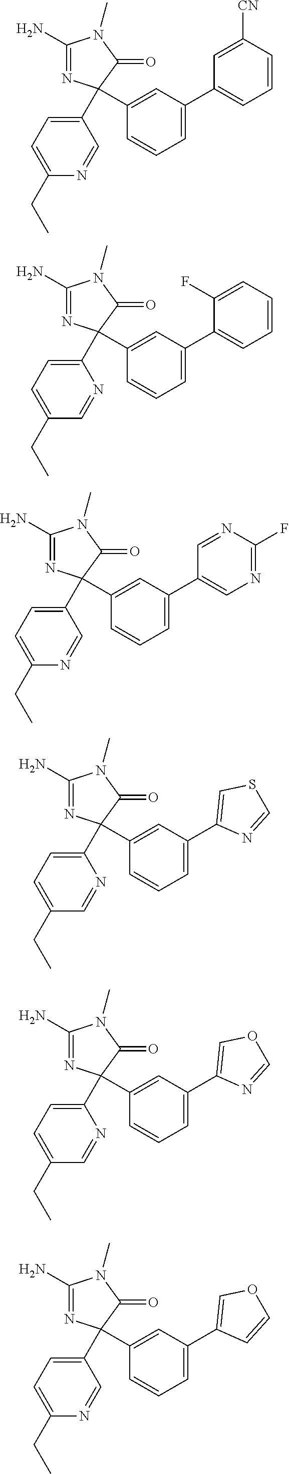

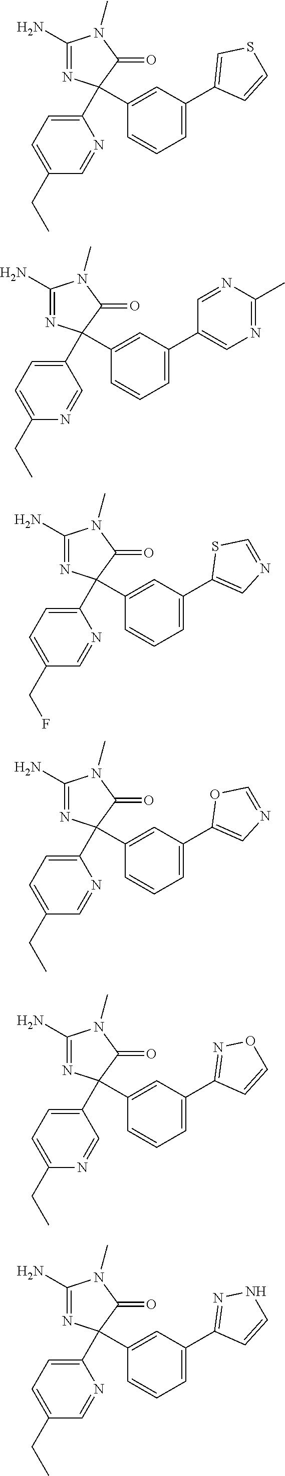

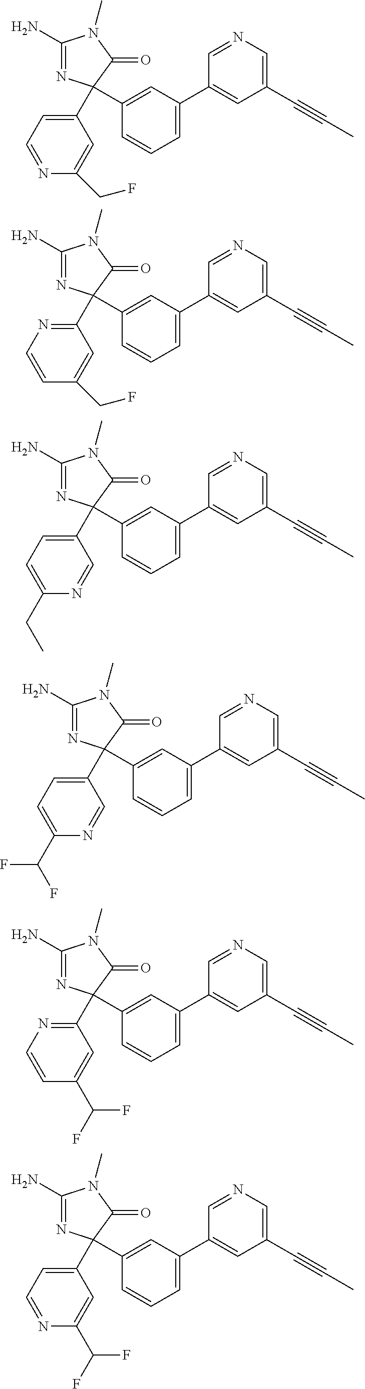

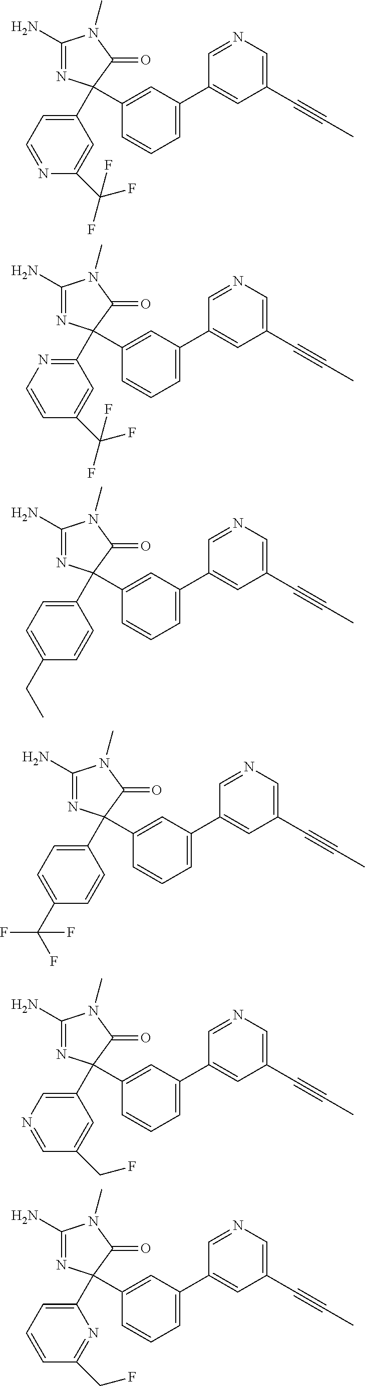

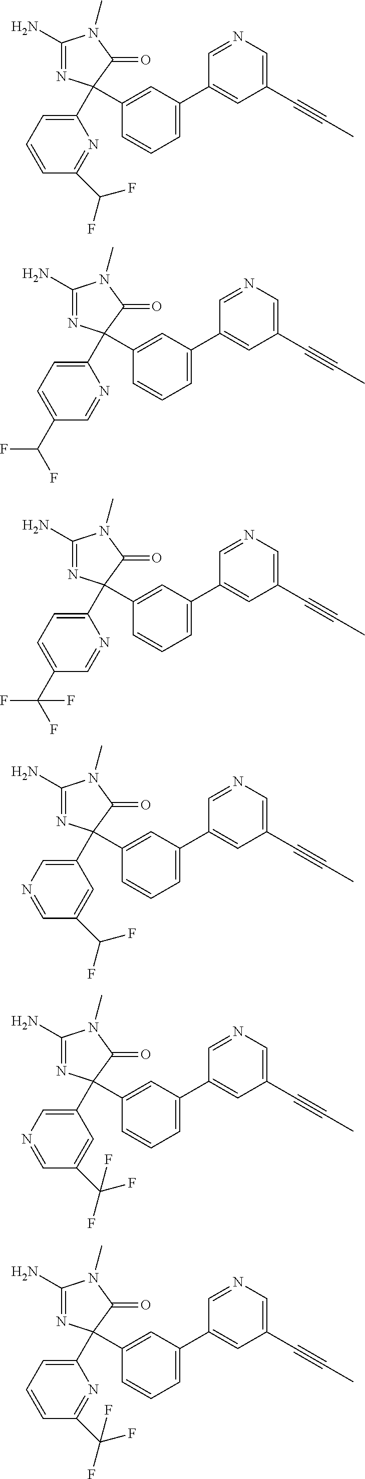

### Compositions

The compounds of the invention can be included in any suitable pharmaceutical composition which can be manufactured by processes well known in the art, e.g., using a variety of well-known tableting, encapsulation, or other oral formulation techniques for oral dosage forms, including one or more physiologically acceptable carriers comprising excipients and auxiliaries which facilitate processing of the active compounds into preparations which can be used pharmaceutically. The compounds of the invention can also be art of parenteral compositions useful for injection, including, without limitation, intravenous, intramuscular and subcutaneous injection. The compounds of the invention may be formulated in aqueous solutions, preferably in physiologically compatible buffers such as physiological saline buffer or polar solvents. The compositions can also be part of lyophilized formulations which can be reconstituted upon need according to techniques know to those of ordinary skill

### Methods of Treatment

The present invention provides a method for treating a disorder in a subject in need thereof with a compound of the present invention, e.g. a compound Formula (I). The method generally includes administering an effective amount of a compound of the invention to the subject. Contemplated disorders treatable with compounds of the invention include those associated with Alzheimer's disease and/or patients in need of cognitive ability enhancement.

The amount of a compound administered which will be considered an effective amount will be dependent upon several factors including the subject or patient being treated, clinical experience, etc. but some aspects of the invention will generally be in the range of from about 0.01 to about 50 mg/kg per day in one or more doses administered orally, parenterally via intravenous, intramuscularly or subcutaneously, transdermally or via inhalation or any other pharmaceutically acceptable route of administration.

### Methods of Preparation

Compounds of formula I may be prepared using conventional synthetic methods and, if required, standard separation or isolation techniques. For example, compounds of formula I may be prepared by reacting a diketone of formula II with an aminoguanidine derivative of formula III at 80° C. in the presence of a base such as a metal carbonate to give the desired formula I compound. The reaction is shown below in Scheme 1.

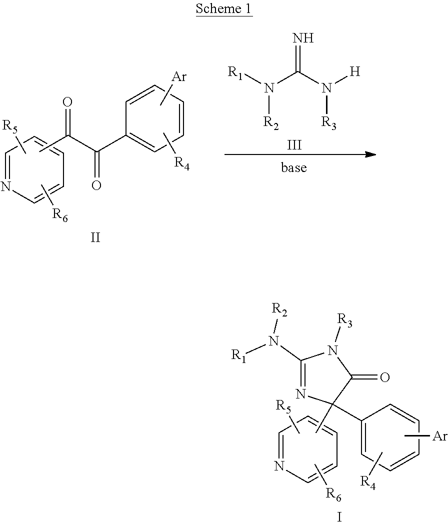

Diketone compounds of formula II may be prepared by reacting an alkyne of formula IV with an oxidizing agent such as Pd(II)Cl₂/DMSO, N-bromosuccinimide/DMSO, ozone, sodium periodate with ruthenium (IV) oxide hydrate, sulfur trioxide, KMnO₄, I₂/DMSO, or combinations thereof, preferable Pd(II)Cl₂/DMSO. The reaction temperature can be around 120 to 145° C. The reaction is shown below in Scheme 2.

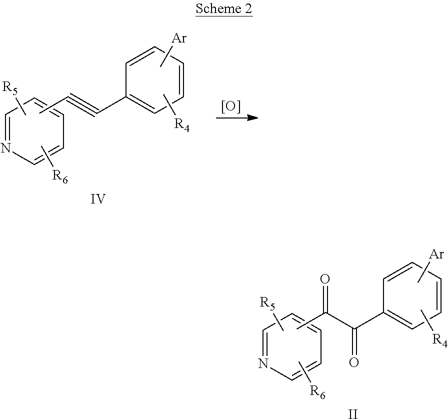

Alkyne compounds of formula IV may be prepared by reacting an ethynylbenzene compound of formula V with a substituted-1-halopyridine compound of formula VI in the presence of a Pd catalyst, such as dichlorobis(triphenylphosphine)palladium (II) and CuI at around 65-75° C. to give the desired pyridineethynylbenzene compound of formula IV. The reaction is shown in Scheme 3 wherein Hal represents Br or I.

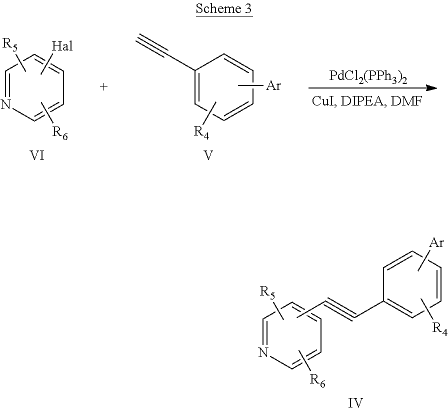

Alkyne compounds of formula V may be prepared by reacting an ethynylmethysilane compound of formula VII with a substituted-1-halobenzene compound of formula VIII in the presence of a Pd catalyst, such as dichlorobis(triphenylphosphine)palladium (II) and CuI at around 65-75° C. to give the desired ethynylbenzene compound of formula V. The reaction is shown in Scheme 4.

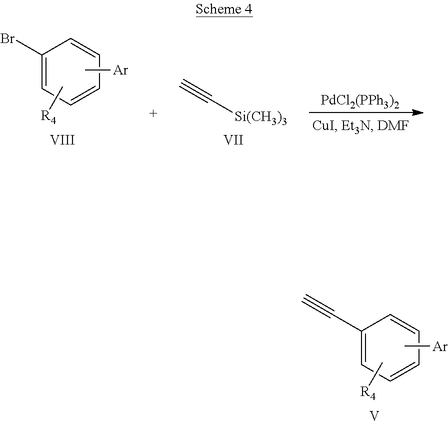

Alkyne compounds of formula IX may be prepared by reacting an ethynylmethysilane compound of formula VII with a substituted-1-halobenzene compound of formula VI in the presence of a Pd catalyst, such as dichlorobis(triphenylphosphine)palladium (II) and CuI at around 65-75° C. to give the desired ethynylbenzene compound of formula IX. The reaction is shown in Scheme 5.

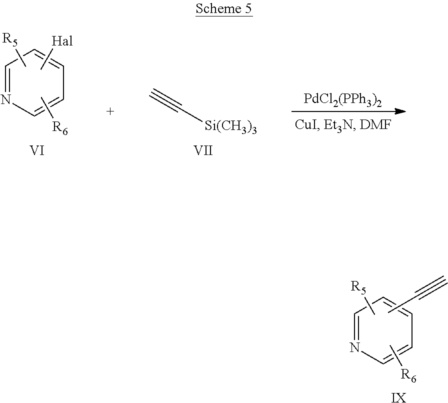

Compounds of formula I can also be prepared by reacting a substituted-1-halobenzene compound of formula X with aryl borane of formula XI in the presence of a Pd catalyst, such as bis(acetonitrile)dichloropalladium(II) and a base such as a metal carbonate at 95° C. to give the desired formula I compound. The reaction is shown below in Scheme 6.

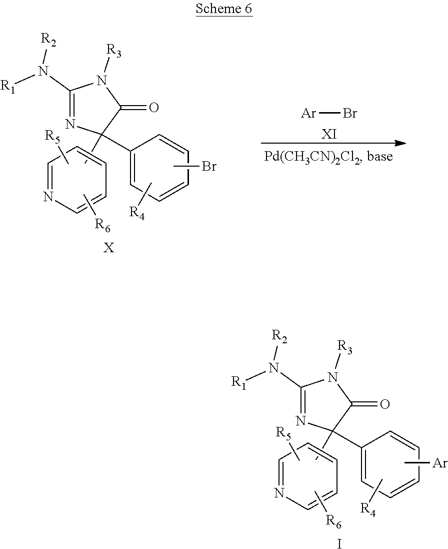

Compounds of formula X may be prepared using the same method described above. The reaction is shown below in Scheme 7 wherein BX₂ represents boronic acid, 9-BBN borane, catechol boronic ester, organotrifluoroborate, MIDA boronate, pinavol boronic ester, 1,8-diaminonaphthyl boronamide, triisopropyl boronate, cyclictriol boranate.

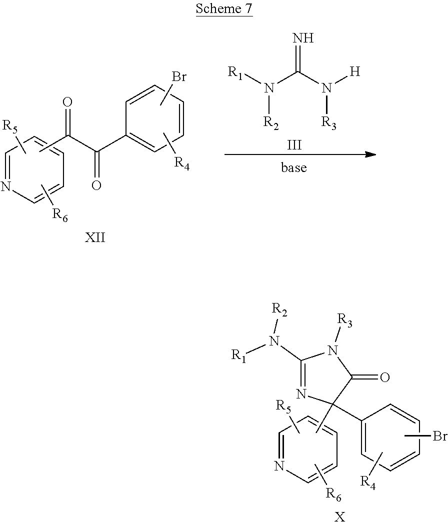

Diketone compounds of formula XII may be prepared using the same method described above. The reaction is shown below in Scheme 8.

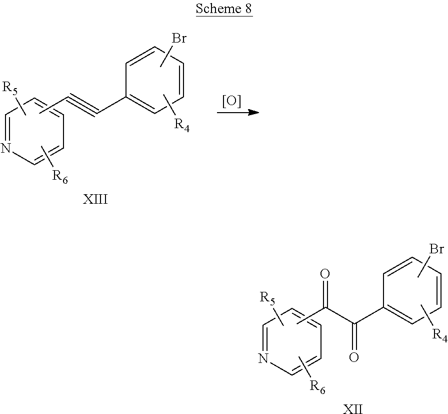

Alkyne compounds of formula XIII may be prepared using the same method described above. The reaction is shown in Scheme 9.

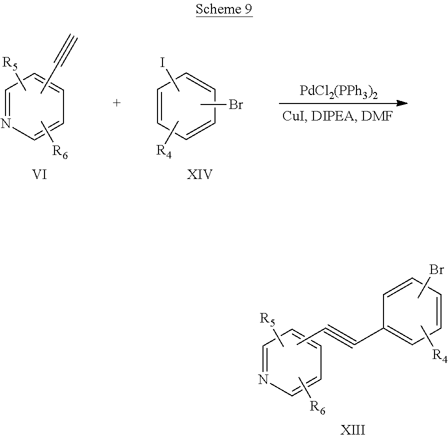

### EXAMPLES

The following examples are provided to further illustrate the present invention but, of course, should not be construed as in any way limiting its scope.

All experiments were performed under anhydrous conditions (i.e. dry solvents) in an atmosphere of argon, except where stated, using oven-dried apparatus and employing standard techniques in handling air-sensitive materials. Aqueous solutions of sodium bicarbonate (NaHCO₃) and sodium chloride (brine) were saturated. The terms DMSO and DMF designate dimethyl sulfoxide and N,N-dimethylformamide, respectively. The terms EtOAc and THF designate ethyl acetate and tetrahydrofuran, respectively.

Analytical thin layer chromatography (TLC) was carried out on Merck Kiesel gel 60 F254 plates with visualization by ultraviolet and/or anisaldehyde, potassium permanganate or phosphomolybdic acid dips.

NMR spectra: 1H Nuclear magnetic resonance spectra were recorded at 400 MHz. Data are presented as follows: chemical shift, multiplicity (s=singlet, d=doublet, t=triplet, q=quartet, qn=quintet, dd=doublet of doublets, m=multiplet, bs=broad singlet), coupling constant (J/Hz) and integration. Coupling constants were taken and calculated directly from the spectra and are uncorrected.

Low resolution mass spectra: Electrospray (ES+) ionization was used. The protonated parent ion (M+H) or parent sodium ion (M+Na) or fragment of highest mass is quoted. Analytical gradient consisted of 10% ACN in water ramping up to 100% ACN over 5 minutes unless otherwise stated.

High performance liquid chromatography (HPLC) was use to analyze the purity of derivatives. HPLC was performed on a Phenomenex Synergi Polar-RP, 4 u, 80 A, 150×4.6 mm column using a Shimadzu system equipped with SPD-M10A Phosphodiode Array Detector. Mobile phase A was water and mobile phase B was acetonitrile with a gradient from 20% to 80% B over 60 minutes and re-equilibrate at A/B (80:20) for 10 minutes. UV detection was at 220 and 54 nm.

### Intermediate I1

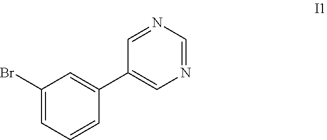 [SMILES: Brc1cccc(-c2cncnc2)c1]

To a solution of pyrimidine-5-boronic acid (4.0 g, 32.2 mmol) in H₂O:EtOH:Toluene (80.0 mL, 16:32:32 mL) was added 1-bromo-3iodobenzene (4.1 mL, 32.2 mmol), potassium carbonate (8.9 g, 64.4 mmol) and Pd(dppf)Cl₂ (2.4 g, 3.2 mmol). The reaction mixture was stirred at 70° C. for 4 hour. The reaction was partitioned with EtOAc (400 mL) and H₂O (200 mL). The organic phase was separated and washed with brine twice, then filtered over a pad of celite. Filtrate was partitioned in seperatory funnel and organic phase was washed with brine, dried over Na₂SO₄, filtered and concentrated. The resulting crude product was purified by Teledyne-Isco flash system by using EtOAc/Hexane, 0 to 30% of ethyl acetate in hexane to provide compound I1 as light pink solid (6.4 g, 82% yield). ¹H NMR (400 MHz, DMSO-d₆) δ (ppm): 9.21 (s, 1H), 9.17 (s, 1H), 8.06 (s, 1H), 7.83 (d, 1H), 7.68 (s, 1H), 7.50 (t, 1H).

### Intermediate I2

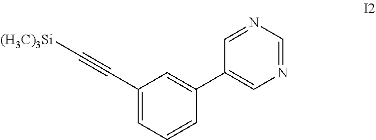

To a solution of 5-(3-bromophenyl)pyrimidine (2.0 g, 8.51 mmol), ethynyltrimethylsilane (1.8 mL, 12.76 mmol) and triethylamine (5.9 mL, 42.55 mmol) in DMF (30.0 mL) was degassed by bubbling with argon for 30 min treated with Pd(PPh₃)₂Cl₂ (0.30 g, 0.43 mmol) with continued argon bubbling, treated CuI (0.16 g, 0.85 mmol), warmed to 65° C. overnight. Reaction mixture was concentrated to dryness and the resulting crude product was purified by Teledyne-Isco flash system by using Hexane/EtOAc, 0 to 15% of hexane in ethyl acetate to provide compound I2 as light yellow solid (1.72 g, 80% yield). ¹H NMR (400 MHz, DMSO-d₆) δ (ppm): 9.20 (s, 1H), 9.17 (s, 2H), 7.91 (s, 1H), 7.86-7.83 (m, 1H), 7.56-7.54 (m, 2H), 0.25 (s, 9H); MS (ESI): Calcd. for C15H16N2Si: 252, found 253 (M+H)⁺.

### Intermediate I3

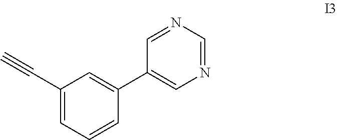

To a solution of 5-(3-((trimethylsilyl)ethynyl)phenyl)pyrimidine (1.7 g, 6.7 mmol) in MeOH (20.0 mL) was added K₂CO₃ (4.6 g, 33.5 mmol). The reaction mixture was filtrated and filtrate was concentrated to provide compound I3 as light brown solid (1.2 g, 99% yield). ¹H NMR (400 MHz, DMSO-d₆) δ (ppm): 9.21 (s, 1H), 9.17 (s, 2H), 7.94 (s, 1H), 7.87-7.85 (dd, 1H), 7.58-7.56 (m, 2H), 4.30 (s, 1H). Calcd. for Cl2H8N2: 180, found 181 (M+H)⁺.

### Intermediate I4

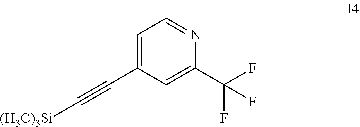

To a solution of 4-bromo-2-(trifluoromethyl)pyridine (2.0 g, 8.85 mmol), ethynyltrimethylsilane (1.8 mL, 13.27 mmol) and triethylamine (6.2 mL, 44.25 mmol) in DMF (20.0 mL) was degassed by bubbling with argon for 30 min treated with Pd(PPh₃)₂Cl₂ (0.18 g, 0.44 mmol) with continued argon bubbling, treated CuI (0.17 g, 0.89 mmol), warmed to 65° C. overnight. Reaction mixture was cooled to room temperature and partitioned between 1M HCl and EtOAc. The organic layer was washed with brine, dried over Na₂SO₄, filtered and concentrated. The suspension was concentrated to dryness and the resulting crude product was purified by Teledyne-Isco flash system by using Hexane 100% to provide compound I4 as light yellow liquid (1.3 g, 61% yield). ¹H NMR (400 MHz, DMSO-d₆) δ (ppm): 8.78 (d, 1H), 7.94 (s, 1H), 7.76 (d, 1H), 0.28 (s, 9H); MS (ESI): Calcd. for C11H12F3NSi: 208, found 210 (M+H)⁺.

### Intermediate I5

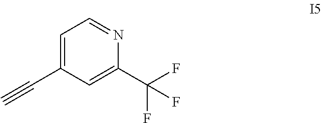

To a solution of 2-(trifluoromethyl)-4-((trimethylsilyl)ethynyl)pyridine (1.3 g, 5.2 mmol) in THF (25.0 mL) was added K₂CO₃ (7.2 g, 52.0 mmol). The reaction mixture was stirred at room temperature for 2 days, and then partitioned between ethyl acetate and water. The organic layer was separated dried over Na₂SO₄, filtered and concentrated to provide compound I5 as light yellow liquid (0.6 g, 79% yield). ¹H NMR (400 MHz, DMSO-d₆) δ (ppm): 8.79 (d, 1H), 7.98 (s, 1H), 7.80 (d, 1H), 4.86 (s, 1H).

### Intermediate I6

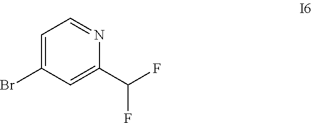

To a solution of 4-bromopicolinaldehyde (10 g, 53.76 mmol) in CHCl₃ (200.0 mL) was added diethylaminosulfur trifluoride (8.5 mL, 64.51 mmol) at 0° C. under argon. The reaction mixture was stirred overnight while the temperature was raised to room temperature. The reaction was quenched by addition of aqueous NaHCO₃ and further diluted with CH₂Cl₂. The solids were filtered off through a pad of celite. The organic layer was separated and aqueous phase was extracted with CH₂Cl₂ (3×). The organic phase dried over Na₂SO₄. The suspension was concentrated to dryness and the resulting crude product was purified by Teledyne-Isco flash system by using Hexane/EtOAc, 0 to 20% of ethyl acetate in hexane to provide compound I6 as light yellow liquid (3.5 g, 32% yield). ¹H NMR (400 MHz, DMSO-d₆) δ (ppm): 8.79 (d, 1H), 7.98 (s, 1H), 7.80 (d, 1H), 4.86 (s, 1H).

### Intermediate I7

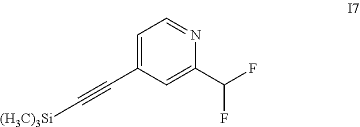

To a solution of 4-bromo-2-(difluoromethyl)pyridine (2.5 g, 12.02 mmol), ethynyltrimethylsilane (2.5 mL, 18.03 mmol) and triethylamine (8.4 mL, 60.10 mmol) in DMF (30.0 mL) was degassed by bubbling with argon for 30 min treated with Pd(PPh₃)₂Cl₂ (0.42 g, 0.60 mmol) with continued argon bubbling, treated CuI (0.23 g, 1.20 mmol), warmed to 65° C. overnight. Reaction mixture was cooled to room temperature and partitioned between 1M HCl and EtOAc. The organic layer was washed with brine, dried over Na₂SO₄, filtered and concentrated. The suspension was concentrated to dryness and the resulting crude product was purified by Teledyne-Isco flash system by using Hexane/EtOAc, 0 to 5% of hexane in ethyl acetate to provide compound I7 as light yellow liquid (1.85 g, 69% yield). ¹H NMR (400 MHz, DMSO-d₆) δ (ppm): 8.70 (d, 1H), 7.69 (s, 1H), 7.61 (d, 1H), 7.09-6.81 (m, 1H), 0.26 (s, 9H); MS (ESI): Calcd. for C11H13F2NSi: 225, found 226 (M+H)⁺.

### Intermediate I8

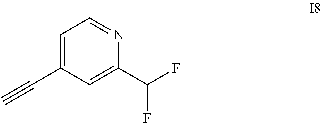

To a solution of 2-(difluoromethyl)-4-((trimethylsilyl)ethynyl)pyridine (1.8 g, 8.0 mmol) in THF (35.0 mL) was added K₂CO₃ (11.1 g, 80.0 mmol). The reaction mixture was stirred at room temperature for 2 days, and then partitioned between ethyl acetate and water. The organic layer was separated dried over Na₂SO₄, filtered and concentrated to provide compound I8 as light yellow liquid (1.0 g, 82% yield). ¹H NMR (400 MHz, DMSO-d₆) δ (ppm): 8.71 (d, 1H), 7.73 (s, 1H), 7.65 (d, 1H), 7.10-6.83 (m, 1H), 4.76 (s, 1H).

### Intermediate I9

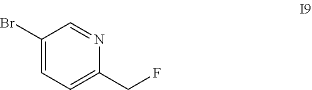

To a solution of (5-bromopyridin-2-yl)methanol (1.0 g, 5.32 mmol) in CH₂Cl₂ (5.0 mL) was added dropwise at −78° C. to a cooled solution of diethylaminosulfur trifluoride (2.1 mL, 15.96 mmol) in CH₂Cl₂ (5.0 mL). The reaction mixture was stirred 1 hour at −78° C. then 1 hour at room temperature. An additional 1 mL of diethylaminosulfur trifluoride was slowly added at −60° C. and reaction was stirred at room temperature overnight. The reaction was quenched with water and the organic layer extracted with CH₂Cl₂, dried over Na₂SO₄. The suspension was concentrated to dryness and the resulting crude product was purified by Teledyne-Isco flash system by using Hexane/EtOAc, 0 to 10% of ethyl acetate in hexane to provide compound I9 as brown solid (90 mg, 9% yield). ¹H NMR (400 MHz, DMSO-d₆) δ (ppm): 8.73 (s, 1H), 8.16-8.14 (m, 1H), 7.51-7.48 (m, 1H), 5.52 (d, 1H), 5.40 (d, 1H); MS (ESI): Calcd. for C6H5BrFN: 190, found 191 (M+H)⁺.

### Intermediate I10

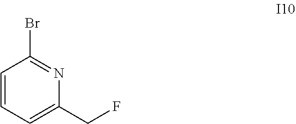

To a solution of (6-bromopyridin-2-yl)methanol (1.0 g, 5.32 mmol) in CH₂Cl₂ (5.0 mL) was added dropwise at −78° C. to a cooled solution of diethylaminosulfur trifluoride (2.1 mL, 15.96 mmol) in CH₂Cl₂ (5.0 mL). The reaction mixture was stirred 1 hour at −78° C. then 1 hour at room temperature. An additional 1 mL of diethylaminosulfur trifluoride was slowly added at −60° C. and reaction was stirred at room temperature overnight. The reaction was quenched with water and the organic layer extracted with CH₂Cl₂, dried over Na₂SO₄. The suspension was concentrated to dryness and the resulting crude product was purified by Teledyne-Isco flash system by using Hexane/EtOAc, 0 to 10% of ethyl acetate in hexane to provide compound I10 as white solid (760 mg, 75% yield). ¹H NMR (400 MHz, DMSO-d₆) δ (ppm): 7.87-7.53 (m, 3H), 5.52 (d, 1H), 5.41 (d, 1H); MS (ESI): Calcd. for C6H5BrFN: 190, found 191 (M+H)⁺.

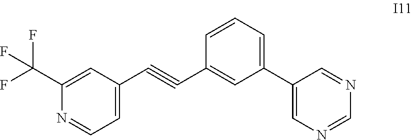 [SMILES: FC(F)(F)c1cc(C#Cc2cccc(-c3cncnc3)c2)ccn1]

### Intermediate I11

4-bromo-2-(trifluoromethyl)pyridine (300 mg, 1.33 mmol), 5-(3-ethynylphenyl)pyrimidine (288 mg, 1.60 mmol) and DIPEA (0.5 mL, 2.66 mmol) in DMF (5.0 mL) was degassed by bubbling with argon for 45 minutes at room temperature, treated with Pd(PPh₃)₂CI₂ (47 mg, 0.07 mmol) with continued argon bubbling, treated with CuI (25 mg, 0.13 mmole) heated at 65° C. overnight. Reaction mixture was concentrated to dryness and the resulting crude product was purified by Teledyne-Isco flash system by using CH₂Cl₂/MeOH, 0 to 5% of methanol in dichloromethane to provide compound I11 as off white solid (330 mg, 76% yield).). ¹H NMR (400 MHz, DMSO-d₆) δ (ppm): 9.22 (d, 3H), 8.85 (d, 1H), 8.15 (s, 1H), 8.10 (s, 1H), 7.98-7.96 (d, 1H), 7.88 (d, 1H), 7.66 (t, 1H); MS (ESI): Calcd. for C18H10F3N3: 325, found 326 (M+H)⁺.

### Intermediate I12

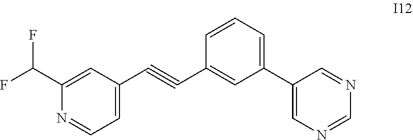 [SMILES: FC(F)c1cc(C#Cc2cccc(-c3cncnc3)c2)ccn1]

4-bromo-2-(difluoromethyl)pyridine (200 mg, 0.96 mmol), 5-(3-ethynylphenyl)pyrimidine (207 mg, 1.15 mmol) and DIPEA (0.3 mL, 1.92 mmol) in DMF (5.0 mL) was degassed by bubbling with argon for 45 minutes at room temperature, treated with Pd(PPh₃)₂CI₂ (34 mg, 0.05 mmol) with continued argon bubbling, treated with CuI (18 mg, 0.10 mmole) heated at 65° C. overnight. Reaction mixture was concentrated to dryness and the resulting crude product was purified by Teledyne-Isco flash system by using CH₂Cl₂/MeOH, 0 to 5% of methanol in dichloromethane to provide compound I12 as light yellow solid (275 mg, 93% yield). ¹H NMR (400 MHz, DMSO-d₆) δ (ppm): 9.23 (d, 3H), 8.77 (d, 1H), 8.14 (s, 1H), 7.97-7.95 (dd, 1H), 7.85 (s, 1H), 7.77-7.74 (m, 2H), 7.66 (t, 1H), 7.15-6.87 (m, 1H); MS (ESI): Calcd. for C18H11F2N3: 307, found 308 (M+H)⁺.

### Intermediate I13

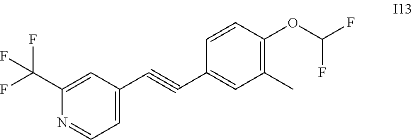 [SMILES: Cc1cc(C#Cc2ccnc(C(F)(F)F)c2)ccc1OC(F)F]

4-bromo-2-(trifluoromethyl)pyridine (300 mg, 1.33 mmol), 1-(difluoromethoxy)-4-ethynyl-2-methylbenzene (290 mg, 1.60 mmol) and DIPEA (0.5 mL, 2.66 mmol) in DMF (5.0 mL) was degassed by bubbling with argon for 45 minutes at room temperature, treated with Pd(PPh₃)₂CI₂ (47 mg, 0.07 mmol) with continued argon bubbling, treated with CuI (25 mg, 0.13 mmole) heated at 65° C. overnight. Reaction mixture was concentrated to dryness and the resulting crude product was purified by Teledyne-Isco flash system by using CH₂Cl₂/MeOH, 0 to 5% of methanol in dichloromethane to provide compound I13 as light yellow solid (340 mg, 78% yield).). ¹H NMR (400 MHz, DMSO-d₆) δ (ppm): 8.81 (d, 1H), 8.04 (s, 1H), 7.83 (d, 1H), 7.62 (s, 1H), 7.57-7.54 (dd, 1H), 7.49-7.12 (m, 2H), 2.25 (s, 3H); MS (ESI): Calcd. for C16H10F5NO: 327, found 328 (M+H)⁺.

### Intermediate I14

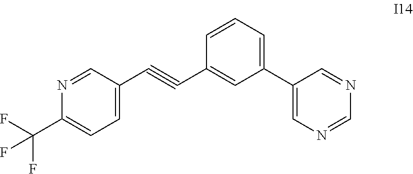 [SMILES: FC(F)(F)c1ccc(C#Cc2cccc(-c3cncnc3)c2)cn1]

5-bromo-2-(trifluoromethyl)pyridine (300 mg, 1.33 mmol), 5-(3-ethynylphenyl)pyrimidine (285 mg, 1.59 mmol) and DIPEA (0.5 mL, 2.66 mmol) in DMF (5.0 mL) was degassed by bubbling with argon for 45 minutes at room temperature, treated with Pd(PPh₃)₂CI₂ (47 mg, 0.07 mmol) with continued argon bubbling, treated with CuI (25 mg, 0.13 mmole) heated at 65° C. overnight. Reaction mixture was concentrated to dryness and the resulting crude product was purified by Teledyne-Isco flash system by using Hexane/EtOAc, 0 to 40% of hexane to provide compound I14 as off white solid (310 mg, 72% yield).). ¹H NMR (400 MHz, DMSO-d₆) δ (ppm): 9.23 (s, 1H), 9.21 (s, 2H), 8.97 (s, 1H), 8.30-8.28 (dd, 1H), 8.12 (s, 1H), 8.01-7.99 (d, 1H), 7.95-7.93 (dd, 1H), 7.75-7.73 (m, 2H), 7.65 (t, 1H); MS (ESI): Calcd. for C18H20F3N3: 325, found 326 (M+H)⁺.

### Intermediate I15

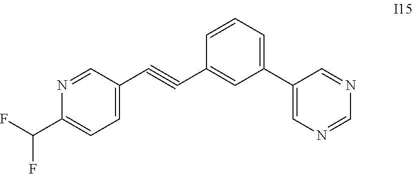 [SMILES: FC(F)c1ccc(C#Cc2cccc(-c3cncnc3)c2)cn1]

5-bromo-2-(difluoromethyl)pyridine (150 mg, 0.72 mmol), 5-(3-ethynylphenyl)pyrimidine (155 mg, 0.86 mmol) and DIPEA (0.25 mL, 1.44 mmol) in DMF (3.0 mL) was degassed by bubbling with argon for 45 minutes at room temperature, treated with Pd(PPh₃)₂CI₂ (25 mg, 0.04 mmol) with continued argon bubbling, treated with CuI (14 mg, 0.07 mmole) heated at 65° C. overnight. Reaction mixture was concentrated to dryness and the resulting crude product was purified by Teledyne-Isco flash system by using Hexane/EtOAc, 0 to 40% of hexane to provide compound I15 as off white solid (165 mg, 74% yield). ¹H NMR (400 MHz, DMSO-d₆) δ (ppm): 9.22 (d, 3H), 8.90 (s, 1H), 7.93 (d, 1H), 7.79 (d, 1H), 7.75-7.72 (dd, 1H), 7.66-7.62 (t, 1H), 7.16-6.89 (m, 1H); MS (ESI): Calcd. for C18H11F2N3: 307, found 308 (M+H)⁺.

### Intermediate I16

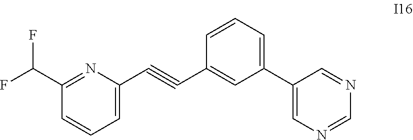 [SMILES: FC(F)c1cccc(C#Cc2cccc(-c3cncnc3)c2)n1]

2-bromo-6-(difluoromethyl)pyridine (150 mg, 0.72 mmol), 5-(3-ethynylphenyl)pyrimidine (155 mg, 0.86 mmol) and DIPEA (0.25 mL, 1.44 mmol) in DMF (3.0 mL) was degassed by bubbling with argon for 45 minutes at room temperature, treated with Pd(PPh₃)₂CI₂ (25 mg, 0.04 mmol) with continued argon bubbling, treated with CuI (14 mg, 0.07 mmole) heated at 65° C. overnight. Reaction mixture was concentrated to dryness and the resulting crude product was purified by Teledyne-Isco flash system by using Hexane/EtOAc, 0 to 50% of hexane to provide compound I16 as off light yellow solid (160 mg, 72% yield). ¹H NMR (400 MHz, DMSO-d₆) δ (ppm): 9.23 (bs, 3H), 8.14-8.08 (m, 2H), 7.94 (d, 1H), 7.86 (d, 1H), 7.80-7.74 (m, 2H), 7.67-7.63 (t, 1H), 7.14-6.87 (m, 1H); MS (ESI): Calcd. for C18H11F2N3: 307, found 308 (M+H)⁺.

### Intermediate I17

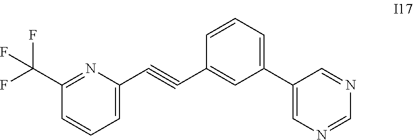 [SMILES: FC(F)(F)c1cccc(C#Cc2cccc(-c3cncnc3)c2)n1]

2-bromo-6-(difluoromethyl)pyridine (150 mg, 0.66 mmol), 5-(3-ethynylphenyl)pyrimidine (144 mg, 0.80 mmol) and DIPEA (0.23 mL, 1.32 mmol) in DMF (3.0 mL) was degassed by bubbling with argon for 45 minutes at room temperature, treated with Pd(PPh₃)₂CI₂ (23 mg, 0.03 mmol) with continued argon bubbling, treated with CuI (13 mg, 0.07 mmole) heated at 65° C. overnight. Reaction mixture was concentrated to dryness and the resulting crude product was used in the next step without purification. Compound I17 was obtained as light brown solid (215 mg). MS (ESI): Calcd. for C18H10F3N3: 325, found 326 (M+H)⁺.

### Intermediate I18

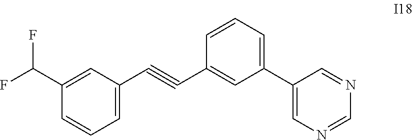 [SMILES: FC(F)c1cccc(C#Cc2cccc(-c3cncnc3)c2)c1]

1-bromo-3-(difluoromethyl)pyridine (150 mg, 0.73 mmol), 5-(3-ethynylphenyl)pyrimidine (157 mg, 0.87 mmol) and DIPEA (0.25 mL, 1.46 mmol) in DMF (3.0 mL) was degassed by bubbling with argon for 45 minutes at room temperature, treated with Pd(PPh₃)₂CI₂ (26 mg, 0.04 mmol) with continued argon bubbling, treated with CuI (14 mg, 0.07 mmole) heated at 65° C. overnight. Reaction mixture was concentrated to dryness and the resulting crude product was purified by Teledyne-Isco flash system by using Hexane/EtOAc, 0 to 40% of hexane to provide compound I18 as light yellow solid (155 mg, 70% yield). ¹H NMR (400 MHz, DMSO-d₆) δ (ppm): 9.22 (d, 3H), 8.07 (s, 1H), 7.91-7.89 (d, 1H), 7.79-7.60 (m, 6H), 7.23-6.95 (m, 1H); MS (ESI): Calcd. for C19H12F2N2: 306, found 307 (M+H)⁺.

### Intermediate I19

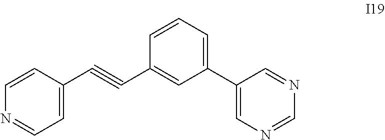 [SMILES: C(#Cc1cccc(-c2cncnc2)c1)c1ccncc1]

4-bromopyridine hydrochloride (150 mg, 0.77 mmol), 5-(3-ethynylphenyl)pyrimidine (157 mg, 0.92 mmol) and DIPEA (0.27 mL, 1.54 mmol) in DMF (3.0 mL) was degassed by bubbling with argon for 45 minutes at room temperature, treated with Pd(PPh₃)₂CI₂ (27 mg, 0.04 mmol) with continued argon bubbling, treated with CuI (15 mg, 0.08 mmole) heated at 65° C. overnight. Reaction mixture was concentrated to dryness and the resulting crude product was purified by Teledyne-Isco flash system by using Hexane/EtOAc, 0 to 80% of hexane to provide compound I19 as off white solid (155 mg, 78% yield). ¹H NMR (400 MHz, DMSO-d₆) δ (ppm): 9.23 (d, 3H), 8.77-8.74 (bs, 1H), 8.12-7.93 (m, 2H), 7.74-7.58 (m, 4H); MS (ESI): Calcd. for C₁₇H₁₁N₃: 257, found 258 (M+H)⁺.

### Intermediate I20

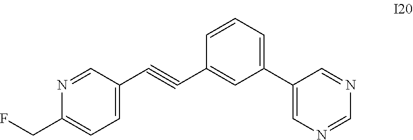 [SMILES: FCc1ccc(C#Cc2cccc(-c3cncnc3)c2)cn1]

5-bromo-2-fluoromethyl pyridine (80 mg, 0.42 mmol), 5-(3-ethynylphenyl)pyrimidine (92 mg, 0.51 mmol) and DIPEA (0.15 mL, 0.84 mmol) in DMF (2.0 mL) was degassed by bubbling with argon for 45 minutes at room temperature, treated with Pd(PPh₃)₂CI₂ (15 mg, 0.02 mmol) with continued argon bubbling, treated with CuI (8 mg, 0.04 mmole) heated at 65° C. overnight. Reaction mixture was concentrated to dryness and the resulting crude product was purified by Teledyne-Isco flash system by using Hexane/EtOAc, 0 to 40% of hexane to provide compound I20 as light yellow solid (45 mg, 37% yield). ¹H NMR (400 MHz, DMSO-d₆) δ (ppm): 9.22 (d, 3H), 8.80 (d, 1H), 8.11-8.08 (m, 2H), 7.92-7.90 (dd, 1H), 7.72-7.58 (m, 3H), 5.59 (s, 1H), 5.47 (s, 1H); MS (ESI): Calcd. for C18H12FN3: 289, found 290 (M+H)⁺.

### Intermediate I21

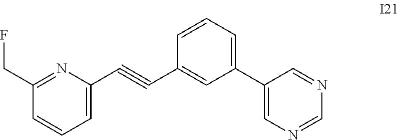 [SMILES: FCc1cccc(C#Cc2cccc(-c3cncnc3)c2)n1]

6-bromo-2-fluoromethyl pyridine (150 mg, 0.79 mmol), 5-(3-ethynylphenyl)pyrimidine (170 mg, 0.95 mmol) and DIPEA (0.28 mL, 1.58 mmol) in DMF (3.0 mL) was degassed by bubbling with argon for 45 minutes at room temperature, treated with Pd(PPh₃)₂CI₂ (28 mg, 0.04 mmol) with continued argon bubbling, treated with CuI (15 mg, 0.08 mmole) heated at 65° C. overnight. Reaction mixture was concentrated to dryness and the resulting crude product was purified by Teledyne-Isco flash system by using Hexane/EtOAc, 0 to 40% of hexane to provide compound I21 as off white solid (190 mg, 83% yield). ¹H NMR (400 MHz, DMSO-d₆) δ (ppm): 9.23 (s, 3H), 8.10 (d, 1H), 8.00-7.92 (m, 2H), 7.75 (d, 1H), 7.69-7.62 (m, 2H), 7.54 (d, 1H), 5.57 (s, 1H), 5.45 (s, 1H); MS (ESI): Calcd. for C18H12FN3: 289, found 290 (M+H)⁺.

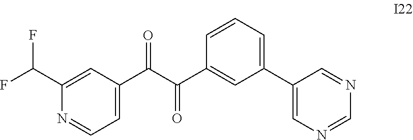 [SMILES: O=C(C(=O)c1ccnc(C(F)F)c1)c1cccc(-c2cncnc2)c1]

### Intermediate I22

5-(3-((2-(difluoromethyl)pyridin-4-yl)ethynyl)phenyl)pyrimidine (300 mg, 0.98 mmol) and dimethylsulfoxide (6.0 mL) was introduced anhydrous argon gas for 15 minutes. Then, bis(acetonitrile)dichloropalladium(II) (26 mg, 0.10 mmol) was added into the mixture and the new mixture was stirred at 145° C. overnight. The mixture cooled to room temperature, poured into water and extracted with EtOAc. The organic extracts were dried over Na₂SO₄. The suspension was concentrated to dryness and the resulting crude product was purified by Teledyne-Isco flash system by using Hexane/EtOAc, 10 to 60% of hexane in ethyl acetate to provide compound I22 as light yellow solid (130 mg, 40% yields). ¹H NMR (400 MHz, DMSO-d₆) δ (ppm): 9.25 (s, 1H), 9.21 (s, 2H), 9.02 (d, 1H), 8.44 (s, 1H), 8.26-8.24 (dd, 1H), 8.16-8.14 (m, 2H), 8.08 (d, 1H), 1.81 (t, 1H), 7.27-7.00 (m, 1H); MS (ESI): Calcd. for C18H11F2N3O2: 339, found 340 (M+H)⁺.

### Intermediate I23

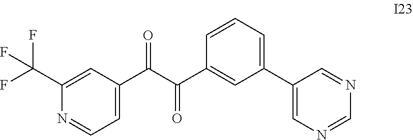 [SMILES: O=C(C(=O)c1ccnc(C(F)(F)F)c1)c1cccc(-c2cncnc2)c1]

5-(3-((2-(trifluoromethyl)pyridin-4-yl)ethynyl)phenyl)pyrimidine (350 mg, 1.08 mmol) and dimethylsulfoxide (6.0 mL) was introduced anhydrous argon gas for 15 minutes. Then, bis(acetonitrile)dichloropalladium(II) (30 mg, 0.11 mmol) was added into the mixture and the new mixture was stirred at 145° C. overnight. The mixture cooled to room temperature, poured into water and extracted with EtOAc. The organic extracts were dried over Na₂SO₄. The suspension was concentrated to dryness and the resulting crude product was purified by Teledyne-Isco flash system by using Hexane/EtOAc, 20 to 60% of hexane in ethyl acetate to provide compound I23 as yellow solid (200 mg, 53% yields). ¹H NMR (400 MHz, DMSO-d₆) δ (ppm): 9.25 (s, 1H), 9.21 (s, 2H), 9.11 (d, 1H), 8.44 (s, 1H), 8.33 (d, 1H), 8.26-8.15 (m, 3H), 7.83-7.79 (t, 1H); MS (ESI): Calcd. for C18H10F3N3O2: 357, found 358 (M+H)⁺.

### Intermediate I24

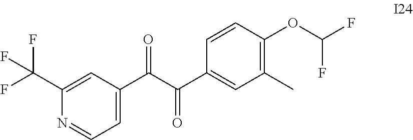 [SMILES: Cc1cc(C(=O)C(=O)c2ccnc(C(F)(F)F)c2)ccc1OC(F)F]

4-((4-(difluoromethoxy)-3-methylphenyl)ethynyl)-2-(trifluoromethyl)pyridine (300 mg, 0.92 mmol) and dimethylsulfoxide (5.0 mL) was introduced anhydrous argon gas for 20 minutes. Then, bis(acetonitrile)dichloropalladium(II) (24 mg, 0.09 mmol) was added into the mixture and the new mixture was stirred at 145° C. overnight. The mixture cooled to room temperature, poured into water and extracted with EtOAc. The organic extracts were dried over Na₂SO₄. The suspension was concentrated to dryness and the resulting crude product was purified by Teledyne-Isco flash system by using Hexane/EtOAc, 0 to 10% of hexane in ethyl acetate to provide compound I24 as yellow solid (205 mg, 62% yields). ¹H NMR (400 MHz, DMSO-d₆) δ (ppm): 9.08 (d, 1H), 8.27 (s, 1H), 8.17 (dd, 1H), 8.03 (d, 1H), 8.00-7.97 (dd, 1H), 7.63-7.26 (m, 2H), 2.30 (s, 3H); MS (ESI): Calcd. for C16H10F5NO3: 359, found 360 (M+H)⁺.

### Intermediate I25

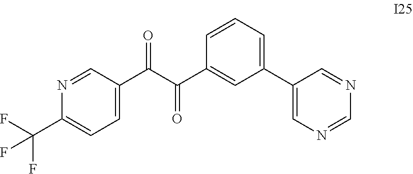 [SMILES: O=C(C(=O)c1cccc(-c2cncnc2)c1)c1ccc(C(F)(F)F)nc1]

5-(3-((6-(trifluoromethyl)pyridin-3-yl)ethynyl)phenyl)pyrimidine (150 mg, 0.46 mmol) and dimethylsulfoxide (3.0 mL) was introduced anhydrous argon gas for 30 minutes. Then, bis(acetonitrile)dichloropalladium(II) (12 mg, 0.05 mmol) was added into the mixture and the new mixture was stirred at 145° C. overnight. The mixture cooled to room temperature, poured into water and extracted with EtOAc. The organic extracts were dried over Na₂SO₄. The suspension was concentrated to dryness and the resulting crude product was purified by Teledyne-Isco flash system by using Hexane/EtOAc, 0 to 60% of hexane to provide compound I25 as light yellow solid (120 mg, 73% yields). ¹H NMR (400 MHz, DMSO-d₆) δ (ppm): 9.33-9.19 (m, 4H), 8.67-8.41 (m, 2H), 8.26-8.13 (m, 3H), 7.84-7.78 (m, 1H); MS (ESI): Calcd. for C18H10F3N3O2: 357, found 358 (M+H)⁺.

### Intermediate I26

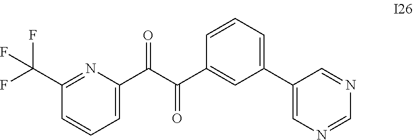 [SMILES: O=C(C(=O)c1cccc(C(F)(F)F)n1)c1cccc(-c2cncnc2)c1]

5-(3-((6-(trifluoromethyl)pyridin-2-yl)ethynyl)phenyl)pyrimidine (200 mg, 0.62 mmol) and dimethylsulfoxide (3.0 mL) was introduced anhydrous argon gas for 30 minutes. Then, bis(acetonitrile)dichloropalladium(II) (16 mg, 0.06 mmol) was added into the mixture and the new mixture was stirred at 145° C. overnight. The mixture cooled to room temperature, poured into water and extracted with EtOAc. The organic extracts were dried over Na₂SO₄. The suspension was concentrated to dryness and the resulting crude product was purified by Teledyne-Isco flash system by using Hexane/EtOAc, 0 to 50% of hexane to provide compound I26 as light yellow solid (60 mg, 27% yields). ¹MS (ESI): Calcd. for C18H10F3N3O2: 357, found 358 (M+H)⁺.

### Intermediate I27

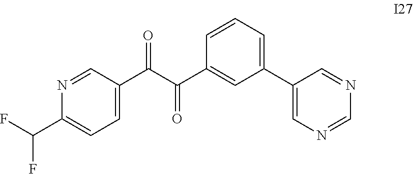 [SMILES: O=C(C(=O)c1cccc(-c2cncnc2)c1)c1ccc(C(F)F)nc1]

5-(3-((6-(difluoromethyl)pyridin-3-yl)ethynyl)phenyl)pyrimidine (150 mg, 0.49 mmol) and dimethylsulfoxide (3.0 mL) was introduced anhydrous argon gas for 30 minutes. Then, bis(acetonitrile)dichloropalladium(II) (13 mg, 0.05 mmol) was added into the mixture and the new mixture was stirred at 145° C. overnight. The mixture cooled to room temperature, poured into water and extracted with EtOAc. The organic extracts were dried over Na₂SO₄. The suspension was concentrated to dryness and the resulting crude product was purified by Teledyne-Isco flash system by using Hexane/EtOAc, 0 to 50% of hexane to provide compound I27 as light yellow solid (100 mg, 60% yields). ¹H NMR (400 MHz, DMSO-d₆) δ (ppm): 9.24 (s, 1H), 9.21 (s, 2H), 8.58 (dd, 1H), 8.42 (s, 1H), 8.26-8.24 (dd, 1H), 8.14 (d, 1H), 7.95 (d, 1H), 7.83-7.79 (t, 1H), 7.25-6.98 (m, 2H); MS (ESI): Calcd. for C18H11F2N3O2: 339, found 340 (M+H)⁺.

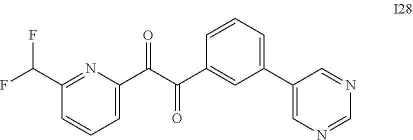 [SMILES: O=C(C(=O)c1cccc(C(F)F)n1)c1cccc(-c2cncnc2)c1]

### Intermediate I28

5-(3-((6-(difluoromethyl)pyridin-2-yl)ethynyl)phenyl)pyrimidine (150 mg, 0.49 mmol) and dimethylsulfoxide (3.0 mL) was introduced anhydrous argon gas for 30 minutes. Then, bis(acetonitrile)dichloropalladium(II) (13 mg, 0.05 mmol) was added into the mixture and the new mixture was stirred at 145° C. overnight. The mixture cooled to room temperature, poured into water and extracted with EtOAc. The organic extracts were dried over Na₂SO₄. The suspension was concentrated to dryness and the resulting crude product was purified by Teledyne-Isco flash system by using Hexane/EtOAc, 0 to 70% of hexane to provide compound I28 as light yellow solid (90 mg, 54% yields). ¹H NMR (400 MHz, DMSO-d₆) δ (ppm): 9.23 (s, 1H), 9.23-9.20 (m, 2H), 8.43-8.35 (m, 3H), 8.24-8.00 (m, 3H), 7.81-7.77 (m, 1H), 7.08-6.80 (m, 1H); ¹MS (ESI): Calcd. for C18H11F2N3O2: 339, found 340 (M+H)⁺.

### Intermediate I29

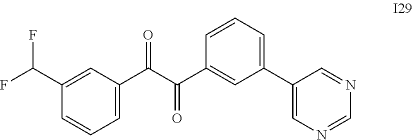 [SMILES: O=C(C(=O)c1cccc(C(F)F)c1)c1cccc(-c2cncnc2)c1]

5-(3-((6-(difluoromethyl)pyridin-3-yl)ethynyl)phenyl)pyrimidine (150 mg, 0.49 mmol) and dimethylsulfoxide (3.0 mL) was introduced anhydrous argon gas for 30 minutes. Then, bis(acetonitrile)dichloropalladium(II) (13 mg, 0.05 mmol) was added into the mixture and the new mixture was stirred at 145° C. overnight. The mixture cooled to room temperature, poured into water and extracted with EtOAc. The organic extracts were dried over Na₂SO₄. The suspension was concentrated to dryness and the resulting crude product was purified by Teledyne-Isco flash system by using Hexane/EtOAc, 0 to 70% of hexane to provide compound I29 as light yellow solid (120 mg, 72% yields). ¹H NMR (400 MHz, DMSO-d₆) δ (ppm): 9.24 (s, 1H), 9.21 (s, 2H), 8.40 (s, 1H), 8.25-8.23 (dd, 1H), 8.17-8.14 (m, 2H), 8.06-8.00 (dd, 2H), 7.82-7.78 (t, 2H), 7.32-7.05 (m, 1H); MS (ESI): Calcd. for C19H12F2N2O2: 338, found 339 (M+H)⁺.

### Intermediate I30

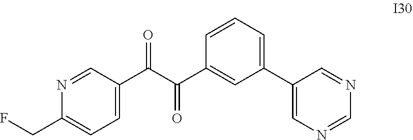 [SMILES: O=C(C(=O)c1cccc(-c2cncnc2)c1)c1ccc(CF)nc1]

5-(3-((6-(fluoromethyl)pyridin-3-yl)ethynyl)phenyl)pyrimidine (40 mg, 0.14 mmol) and dimethylsulfoxide (1.5 mL) was introduced anhydrous argon gas for 30 minutes. Then, bis(acetonitrile)dichloropalladium(II) (5 mg, 0.02 mmol) was added into the mixture and the new mixture was stirred at 145° C. overnight. The mixture cooled to room temperature, poured into water and extracted with EtOAc. The organic extracts were dried over Na₂SO₄. The suspension was concentrated to dryness and the resulting crude product was purified by Teledyne-Isco flash system by using Hexane/EtOAc, 0 to 50% of hexane to provide compound I30 as light yellow solid (40 mg, 91% yields). ¹H NMR (400 MHz, DMSO-d₆) δ (ppm): 9.24 (s, 1H), 9.22 (d, 2H), 9.14 (d, 1H), 8.47-8.44 (dd, 1H), 8.40 (t, 1H), 8.25-8.23 (dd, 1H), 8.11-8.09 (dd, 1H), 7.83-7.72 (m, 2H), 5.69 (s, 1H), 5.58 (s, 1H); MS (ESI): Calcd. for C18H12FN3O2: 321, found 322 (M+H)⁺.

### Intermediate I31

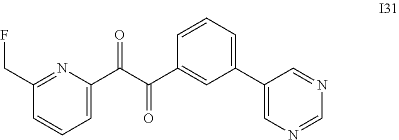 [SMILES: O=C(C(=O)c1cccc(CF)n1)c1cccc(-c2cncnc2)c1]

5-(3-((6-(fluoromethyl)pyridin-2-yl)ethynyl)phenyl)pyrimidine (180 mg, 0.62 mmol) and dimethylsulfoxide (3.0 mL) was introduced anhydrous argon gas for 30 minutes. Then, bis(acetonitrile)dichloropalladium(II) (16 mg, 0.06 mmol) was added into the mixture and the new mixture was stirred at 145° C. overnight. The mixture cooled to room temperature, poured into water and extracted with EtOAc. The organic extracts were dried over Na₂SO₄. The suspension was concentrated to dryness and the resulting crude product was purified by Teledyne-Isco flash system by using Hexane/EtOAc, 0 to 40% of hexane to provide compound I33 as light yellow solid (86 mg, 43% yields). ¹H NMR (400 MHz, DMSO-d₆) δ (ppm): 9.23 (s, 1H), 9.19 (s, 2H), 8.33-8.20 (m, 4H), 7.97-7.95 (dd, 1H), 7.89-7.87 (dd, 1H), 7.79-7.76 (t, 1H), 5.49 (s, 1H), 5.37 (s, 1H); MS (ESI): Calcd. for C8H12FN3O2: 321, found 322 (M+H)⁺.

### Preparation of Exemplary Compounds

### Example 1

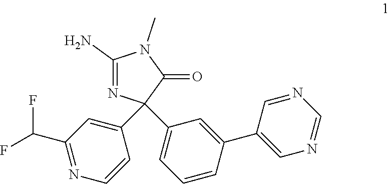 [SMILES: CN1C(=O)C(c2cccc(-c3cncnc3)c2)(c2ccnc(C(F)F)c2)N=C1N]

To a solution of 1-(2-(difluoromethyl)pyridin-4-yl)-2-(3-(pyrimidin-5-yl)phenyl)ethane-1,2-dione (120 mg, 0.35 mmol) in isopropyl alcohol (3.0 mL) was added 1-methylguanidine HCl (40 mg, 0.53 mmol) and sodium carbonate (56 mg, 0.53 mmol) at room temperature and stirred at 80° C. overnight. Reaction mixture was filtered. The suspension was concentrated to dryness and the resulting crude product was purified by Teledyne-Isco flash system by using CH₂Cl₂/MeOH, 0 to 10% of methanol in dichloromethane to provide compound 1 as light yellow solid (100 mg, 71% yield). ¹H NMR (400 MHz, DMSO-d₆) δ (ppm): 9.20 (s, 1H), 9.03 (s, 2H), 8.64 (d, 1H), 7.82 (d, 2H), 7.74-7.70 (m, 2H), 7.59-7.50 (m, 2H), 7.09-6.81 (m, 3H), 3.02 (s, 3H); ¹MS (ESI): Calcd. for C20H16F2N6O: 394, found 395 (M+H)⁺. HPLC: retention t: 6.74 min. purity: 95%.

### Example 2

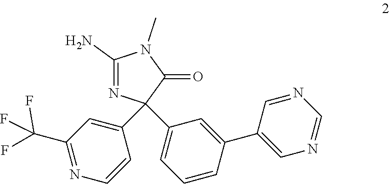 [SMILES: CN1C(=O)C(c2cccc(-c3cncnc3)c2)(c2ccnc(C(F)(F)F)c2)N=C1N]

To a solution of 1-(3-(pyrimidin-5-yl)phenyl)-2-(2-(trifluoromethyl)pyridin-4-yl)ethane-1,2-dione (180 mg, 0.50 mmol) in isopropyl alcohol (5.0 mL) was added 1-methylguanidine HCl (56 mg, 0.76 mmol) and sodium carbonate (80 mg, 0.76 mmol) at room temperature and stirred at 80° C. overnight. Reaction mixture was filtered. The suspension was concentrated to dryness and the resulting crude product was purified by Teledyne-Isco flash system by using CH₂Cl₂/MeOH, 0 to 10% of methanol in dichloromethane to provide compound 2 as light yellow solid (100 mg, 71% yield). ¹H NMR (400 MHz, DMSO-d₆) δ (ppm): 9.20 (s, 1H), 9.04 (s, 2H), 8.74 (d, 1H), 7.95 (s, 1H), 7.89 (d, 1H), 7.83 (d, 1H), 7.72-7.51 (m, 3H), 6.98 (bs, 2H), 3.02 (s, 3H); ¹MS (ESI): Calcd. for C20H15F3N6O: 412, found 413 (M+H)⁺. HPLC: retention time: 8.95 min. purity: 99%.

### Example 3

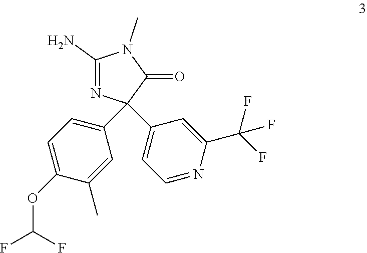 [SMILES: Cc1cc(C2(c3ccnc(C(F)(F)F)c3)N=C(N)N(C)C2=O)ccc1OC(F)F]

To a solution of 1-(4-(difluoromethoxy)-3-methylphenyl)-2-(2-(trifluoromethyl)pyridin-4-yl)ethane-1,2-dione (200 mg, 0.56 mmol) in isopropyl alcohol (5.0 mL) was added 1-methylguanidine HCl (62 mg, 0.84 mmol) and sodium carbonate (89 mg, 0.84 mmol) at room temperature and stirred at 80° C. overnight. Reaction mixture was filtered. The suspension was concentrated to dryness and the resulting crude product was purified by Teledyne-Isco flash system by using CH₂Cl₂/MeOH, 0 to 10% of methanol in dichloromethane to provide compound 3 as off white solid (155 mg, 67% yield). ¹H NMR (400 MHz, DMSO-d₆) δ (ppm): 8.72 (d, 1H), 7.89 (s, 1H), 7.81 (d, 1H), 7.38-7.10 (m, 4H), 6.94-6.92 (m, 2H), 2.99 (s, 3H), 2.18 (s, 3H); ¹MS (ESI): Calcd. for C18H15F5N4O2: 414, found 415 (M+H)⁺. HPLC: retention time: 16.24 min. purity: 99%.

### Example 4

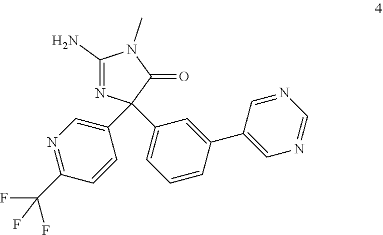 [SMILES: CN1C(=O)C(c2ccc(C(F)(F)F)nc2)(c2cccc(-c3cncnc3)c2)N=C1N]

To a solution of 1-(3-(pyrimidin-5-yl)phenyl)-2-(6-(trifluoromethyl)pyridin-3-yl)ethane-1,2-dione (100 mg, 0.28 mmol) in isopropyl alcohol (3.0 mL) was added 1-methylguanidine HCl (31 mg, 0.42 mmol) and sodium carbonate (45 mg, 0.42 mmol) at room temperature and stirred at 80° C. overnight. Reaction mixture was filtered. The suspension was concentrated to dryness and the resulting crude product was purified by Teledyne-Isco flash system by using CH₂Cl₂/MeOH, 0 to 10% of methanol in dichloromethane to provide compound 4 as light yellow solid (56 mg, 49% yield). ¹H NMR (400 MHz, DMSO-d₆) δ (ppm): 9.19 (s, 1H), 9.04 (s, 2H), 8.87 (d, 1H), 8.20-8.17 (dd, 1H), 7.89-7.83 (m, 2H), 7.72-7.50 (m, 3H), 6.92 (bs, 2H), 3.02 (s, 3H); ¹MS (ESI): Calcd. for C20H15F3N6O: 412, found 413 (M+H)⁺. HPLC: retention time: 9.94 min. purity: 99.9%.

### Example 5

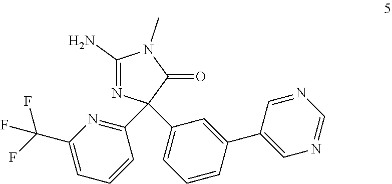 [SMILES: CN1C(=O)C(c2cccc(-c3cncnc3)c2)(c2cccc(C(F)(F)F)n2)N=C1N]

To a solution of 1-(3-(pyrimidin-5-yl)phenyl)-2-(6-(trifluoromethyl)pyridin-2-yl)ethane-1,2-dione (50 mg, 0.14 mmol) in isopropyl alcohol (3.0 mL) was added 1-methylguanidine HCl (15 mg, 0.21 mmol) and sodium carbonate (80 mg, 0.21 mmol) at room temperature and stirred at 80° C. overnight. Reaction mixture was filtered. The suspension was concentrated to dryness and the resulting crude product was purified by Teledyne-Isco flash system by using CH₂Cl₂/MeOH, 0 to 10% of methanol in dichloromethane to provide compound 5 as light yellow solid (43 mg, 74% yield). ¹H NMR (400 MHz, DMSO-d₆) δ (ppm): 9.20-9.04 (m, 3H), 8.04-8.00 (m, 2H), 7.87-7.73 (m, 3H), 7.57-7.51 (m, 2H), 6.84-6.81 (bs, 2H), 3.00 (s, 3H); ¹MS (ESI): Calcd. for C20H15F3N6O: 412, found 413 (M+H)⁺. HPLC: retention time: 11.02 min. purity: 97%.

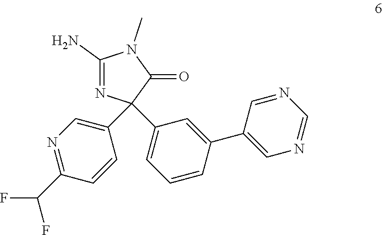 [SMILES: CN1C(=O)C(c2ccc(C(F)F)nc2)(c2cccc(-c3cncnc3)c2)N=C1N]

### Example 6

To a solution of 1-(6-(difluoromethyl)pyridin-3-yl)-2-(3-(pyrimidin-5-yl)phenyl)ethane-1,2-dione (150 mg, 0.49 mmol) in isopropyl alcohol (5.0 mL) was added 1-methylguanidine HCl (53 mg, 0.73 mmol) and sodium carbonate (77 mg, 0.73 mmol) at room temperature and stirred at 80° C. overnight. Reaction mixture was filtered. The suspension was concentrated to dryness and the resulting crude product was purified by Teledyne-Isco flash system by using CH₂Cl₂/MeOH, 0 to 10% of methanol in dichloromethane to provide compound 6 as light yellow solid (80 mg, 42% yield). ¹H NMR (400 MHz, DMSO-d₆) δ (ppm): 9.20 (s, 1H), 9.03 (s, 2H), 8.78 (d, 1H), 8.10-8.08 (dd, 1H), 7.82 (d, 1H), 7.71-7.66 (m, 2H), 7.59-7.50 (m, 2H), 7.06-6.79 (m, 3H), 3.02 (s, 3H); ¹MS (ESI): Calcd. for C20H16F2N6O: 394, found 395 (M+H)⁺. HPLC: retention time: 7.27 min. purity: 99.7%.

### Example 7

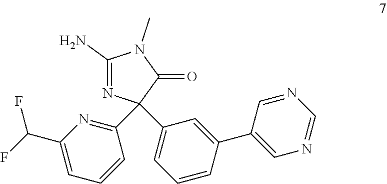 [SMILES: CN1C(=O)C(c2cccc(-c3cncnc3)c2)(c2cccc(C(F)F)n2)N=C1N]

To a solution of 1-(6-(difluoromethyl)pyridin-2-yl)-2-(3-(pyrimidin-5-yl)phenyl)ethane-1,2-dione (80 mg, 0.24 mmol) in isopropyl alcohol (3.0 mL) was added 1-methylguanidine HCl (40 mg, 0.36 mmol) and sodium carbonate (38 mg, 0.36 mmol) at room temperature and stirred at 80° C. overnight. Reaction mixture was filtered. The suspension was concentrated to dryness and the resulting crude product was purified by Teledyne-Isco flash system by using CH₂Cl₂/MeOH, 0 to 10% of methanol in dichloromethane to provide compound 7 as light yellow solid (90 mg, 97% yield). ¹H NMR (400 MHz, DMSO-d₆) δ (ppm): 9.20 (d, 1H), 9.03 (d, 2H), 8.00 (s, 1H), 7.95-7.72 (m, 3H), 7.60-7.35 (m, 3H), 7.01-6.74 (m, 3H), 3.00 (s, 3H); ¹MS (ESI): Calcd. for C20H16F2N6O: 394, found 395 (M+H)⁺. HPLC: retention time: 9.09 min. purity: 98%.

### Example 8

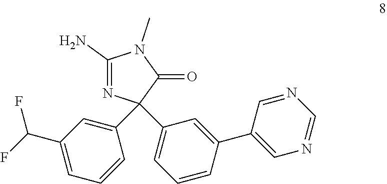 [SMILES: CN1C(=O)C(c2cccc(-c3cncnc3)c2)(c2cccc(C(F)F)c2)N=C1N]

To a solution of 1-(3-(difluoromethyl)phenyl)-2-(3-(pyrimidin-5-yl)phenyl)ethane-1,2-dione (100 mg, 0.30 mmol) in isopropyl alcohol (5.0 mL) was added 1-methylguanidine HCl (48 mg, 0.44 mmol) and sodium carbonate (47 mg, 0.44 mmol) at room temperature and stirred at 80° C. overnight. Reaction mixture was filtered. The suspension was concentrated to dryness and the resulting crude product was purified by Teledyne-Isco flash system by using CH₂Cl₂/MeOH, 0 to 10% of methanol in dichloromethane to provide compound 8 as light yellow solid (95 mg, 82% yield). ¹H NMR (400 MHz, DMSO-d₆) δ (ppm): 9.20 (s, 1H), 9.01 (s, 2H), 7.80 (s, 1H), 7.77-7.67 (m, 3H), 7.56-7.44 (m, 4H), 7.17-6.81 (m, 3H), 3.01 (s, 3H); ¹MS (ESI): Calcd. for C21H17F2N5O: 393, found 394 (M+H)⁺. HPLC: retention time: 10.73 min. purity: 98%.

### Example 9

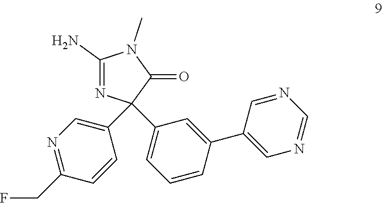 [SMILES: CN1C(=O)C(c2ccc(CF)nc2)(c2cccc(-c3cncnc3)c2)N=C1N]

To a solution of 1-(6-(fluoromethyl)pyridin-3-yl)-2-(3-(pyrimidin-5-yl)phenyl)ethane-1,2-dione (50 mg, 0.16 mmol) in isopropyl alcohol (2.0 mL) was added 1-methylguanidine HCl (26 mg, 0.24 mmol) and sodium carbonate (25 mg, 0.24 mmol) at room temperature and stirred at 80° C. overnight. Reaction mixture was filtered. The suspension was concentrated to dryness and the resulting crude product was purified by Teledyne-Isco flash system by using CH₂Cl₂/MeOH, 0 to 10% of methanol in dichloromethane to provide compound 9 off white solid (17 mg, 29% yield). ¹H NMR (400 MHz, DMSO-d₆) δ (ppm): 9.20 (s, 1H), 9.02 (d, 2H), 8.67 (s, 1H), 7.96-7.94 (t, 1H), 7.81 (s, 1H), 7.70 (d, 1H), 7.58-7.46 (m, 3H), 6.86-6.84 (bs, 2H), 5.49 (s, 1H), 5.38 (s, 1H), 3.02 (s, 3H); ¹MS (ESI): Calcd. for C20H17FN6O: 376, found 377 (M+H)⁺. HPLC: retention time: 6.51 min. purity: 98%.

### Example 10

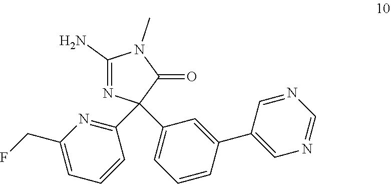 [SMILES: CN1C(=O)C(c2cccc(-c3cncnc3)c2)(c2cccc(CF)n2)N=C1N]

To a solution of 1-(6-(fluoromethyl)pyridin-2-yl)-2-(3-(pyrimidin-5-yl)phenyl)ethane-1,2-dione (80 mg, 0.25 mmol) in isopropyl alcohol (2.0 mL) was added 1-methylguanidine HCl (42 mg, 0.37 mmol) and sodium carbonate (40 mg, 0.37 mmol) at room temperature and stirred at 80° C. overnight. Reaction mixture was filtered. The suspension was concentrated to dryness and the resulting crude product was purified by Teledyne-Isco flash system by using CH₂Cl₂/MeOH, 0 to 10% of methanol in dichloromethane to provide compound 10 off white solid (75 mg, 80% yield). ¹H NMR (400 MHz, DMSO-d₆) δ (ppm): 9.19 (s, 1H), 9.03 (d, 2H), 7.98-7.97 (t, 1H), 7.80-7.71 (m, 3H), 7.54-7.13 (m, 3H), 6.83-6.79 (bs, 2H), 5.47 (s, 1H), 5.35 (s, 1H), 2.99 (s, 3H); ¹MS (ESI): Calcd. for C20H17FN6O: 376, found 377 (M+H)⁺. HPLC: retention time: 8.56 min. purity: 96%.

The following Examples were prepared in the Table 1 in the same procedure as that in Example E1-E10 with the appreciate intermediates showed as above.

TABLE 1

| Compound ID | Structure | Mass (M) | LCMS (M + H) |
|---|---|---|---|
| 11 | 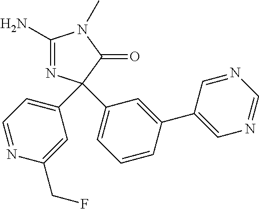 [SMILES: CN1C(=O)C(c2cccc(-c3cncnc3)c2)(c2ccnc(CF)c2)N=C1N] | 376 | 377 |
| 12 | 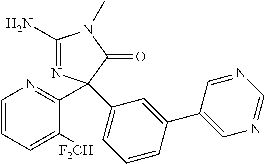 | 394 | 395 |
| 13 | 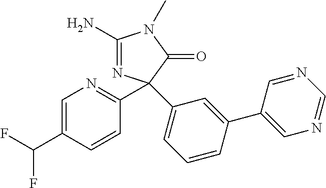 [SMILES: CN1C(=O)C(c2cccc(-c3cncnc3)c2)(c2ccc(C(F)F)cn2)N=C1N] | 394 | 395 |
| 14 | 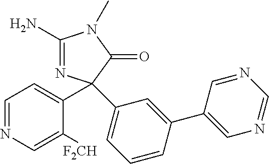 | 394 | 395 |
| 15 | 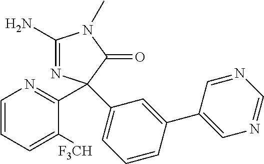 | 413 | 413 |
| 16 | 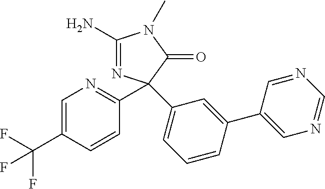 [SMILES: CN1C(=O)C(c2cccc(-c3cncnc3)c2)(c2ccc(C(F)(F)F)cn2)N=C1N] | 413 | 414 |
| 17 | 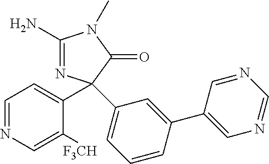 | 413 | 413 |
| 18 | 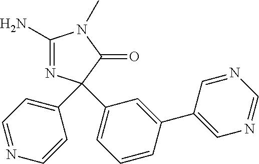 [SMILES: CN1C(=O)C(c2ccncc2)(c2cccc(-c3cncnc3)c2)N=C1N] | 344 | 345 |
| 19 | 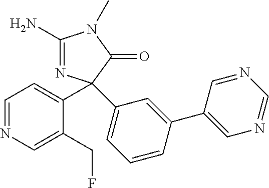 [SMILES: CN1C(=O)C(c2cccc(-c3cncnc3)c2)(c2ccncc2CF)N=C1N] | 376 | 377 |
| 20 | 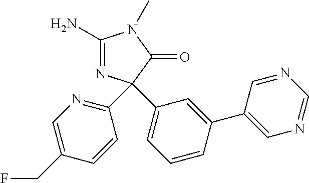 [SMILES: CN1C(=O)C(c2cccc(-c3cncnc3)c2)(c2ccc(CF)cn2)N=C1N] | 376 | 377 |

### Example 21 Inhibition of BACE Cleavage

The compounds were evaluated for inhibition of BACE cleavage of a P5-P5′ fluorescent peptide substrate containing the Swedish mutation cleavage site. In detail, 4 μL of assay buffer was added to each well, followed by 2 μL of BACE-1 diluted in assay buffer to 7.5 ng/uL. Then, 2 μL of compound, at concentrations along an 11 pt. two-fold dilution series starting at 10 uM, or 0.5 uM, were added to the appropriated wells and incubated for 30 minutes at room temperature. For all dilutions of inhibitor, the compound stock solutions of 10 mM in DMSO were diluted to 50 uM in water; subsequent dilutions were in 0.5% DMSO. Afterwards, 2 μL of fluorogenic P5-P5′ BACE-1 substrate, diluted to 50 μM in assay buffer, were added to each well, after which the signal generated was read every 30 minutes at 25° C. for 2 hr. The Table 2 shows the IC₅₀ data.

TABLE 2

| Com- | | IC₅₀ |
|---|---|---|
| pound ID | Structure | (μM) |
| 1 | 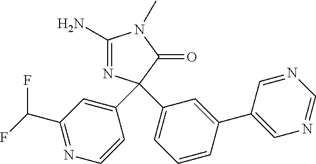 [SMILES: CN1C(=O)C(c2cccc(-c3cncnc3)c2)(c2ccnc(C(F)F)c2)N=C1N] | 0.03 |
| 2 | 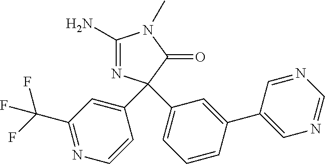 [SMILES: CN1C(=O)C(c2cccc(-c3cncnc3)c2)(c2ccnc(C(F)(F)F)c2)N=C1N] | 0.07 |
| 3 | 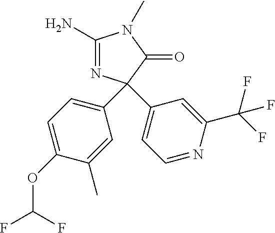 [SMILES: Cc1cc(C2(c3ccnc(C(F)(F)F)c3)N=C(N)N(C)C2=O)ccc1OC(F)F] | 9.72 |
| 4 | 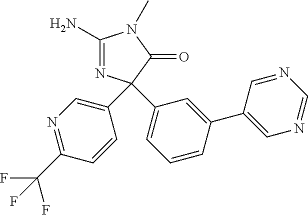 [SMILES: CN1C(=O)C(c2ccc(C(F)(F)F)nc2)(c2cccc(-c3cncnc3)c2)N=C1N] | 0.98 |
| 5 | 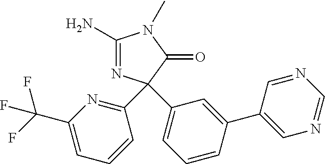 [SMILES: CN1C(=O)C(c2cccc(-c3cncnc3)c2)(c2cccc(C(F)(F)F)n2)N=C1N] | 7.86 |
| 6 | 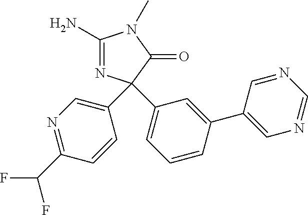 [SMILES: CN1C(=O)C(c2ccc(C(F)F)nc2)(c2cccc(-c3cncnc3)c2)N=C1N] | 0.94 |
| 7 | 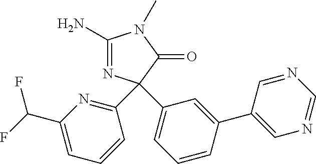 [SMILES: CN1C(=O)C(c2cccc(-c3cncnc3)c2)(c2cccc(C(F)F)n2)N=C1N] | 0.50 |
| 8 | 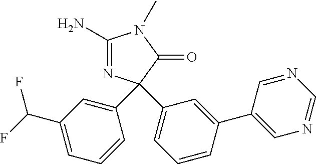 [SMILES: CN1C(=O)C(c2cccc(-c3cncnc3)c2)(c2cccc(C(F)F)c2)N=C1N] | 0.17 |
| 9 | 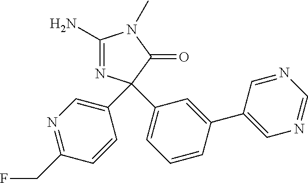 [SMILES: CN1C(=O)C(c2ccc(CF)nc2)(c2cccc(-c3cncnc3)c2)N=C1N] | NA |
| 10 | 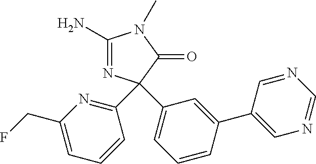 [SMILES: CN1C(=O)C(c2cccc(-c3cncnc3)c2)(c2cccc(CF)n2)N=C1N] | NA |
| 11 | 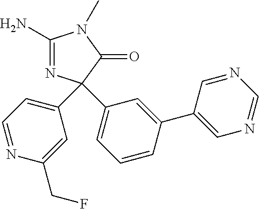 [SMILES: CN1C(=O)C(c2cccc(-c3cncnc3)c2)(c2ccnc(CF)c2)N=C1N] | NA |
| 12 | 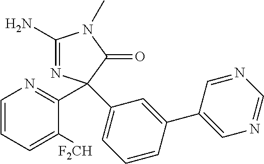 | NA |
| 13 | 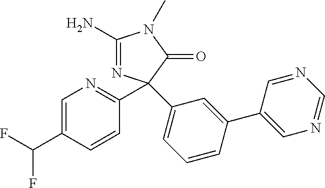 [SMILES: CN1C(=O)C(c2cccc(-c3cncnc3)c2)(c2ccc(C(F)F)cn2)N=C1N] | NA |
| 14 | 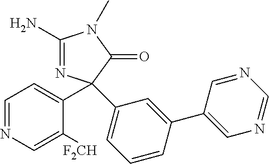 | NA |
| 15 | 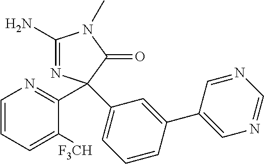 | NA |
| 16 | 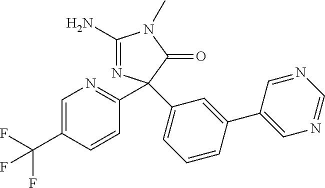 [SMILES: CN1C(=O)C(c2cccc(-c3cncnc3)c2)(c2ccc(C(F)(F)F)cn2)N=C1N] | NA |
| 17 | 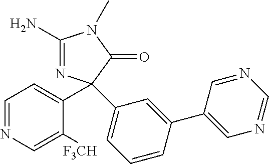 | NA |
| 18 | 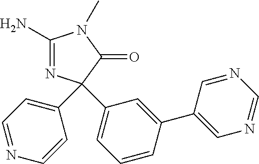 [SMILES: CN1C(=O)C(c2ccncc2)(c2cccc(-c3cncnc3)c2)N=C1N] | NA |
| 19 | 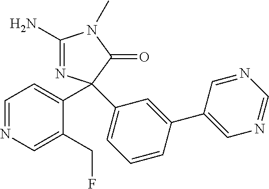 [SMILES: CN1C(=O)C(c2cccc(-c3cncnc3)c2)(c2ccncc2CF)N=C1N] | NA |
| 20 | 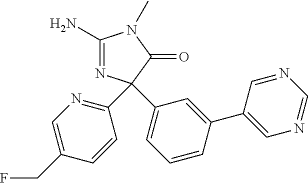 [SMILES: CN1C(=O)C(c2cccc(-c3cncnc3)c2)(c2ccc(CF)cn2)N=C1N] | NA |

1. A compound of the formula: 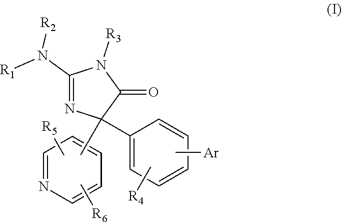
or a pharmaceutically acceptable salt thereof, wherein:
Ar is a) one of phenyl, pyridyl, pyrimidyl, pyrazinyl, imidazolyl, pyrazolyl, thiazolyl, oxazolyl, and isoxazolyl, wherein the Ar is optionally substituted with one or more substituent selected from among halogen, CN, C₁-C₄ alkyl, C₂-C₄ alkenyl, C₂-C₄ alkynyl, C₁-C₄ fluoroalkyl and C₁-C₄ alkoxy; or
b) an optionally substituted heteroaryl group;
R₁ and R₂ are independently hydrogen or a C1-C₄ alkyl;
R₃ is hydrogen or a C1-C6 alkyl, which is optionally substituted with halogen, hydroxy, cyano, amino, —COOH or oxo;
R₄ is selected from the group consisting of hydrogen, halogen, CN, OH, NR₇R₈, CONR₇R₈, C₁-C₄ alkyl, C₂-C₄ alkenyl, C₂-C₄ alkynyl, C₃-C₄ aryl or heteroaryl, (C₃-C₄ cycloalkyl)C1-C₄ alkyl, C₁-C₄ haloalkyl, C₁-C₄ alkoxy, C₁-C₄ alkylthio, C₂-C₄ alkanoyl, C₁-C₄ alkoxycarbonyl, C₂-C₄ alkanoyloxy, C₁-C₄ alkylsulfonyl, mono- and di-(C₁-C₄ alkyl) sulfonamido, and mono- and di-(C1-C₄ alkyl)aminocarbonyl, each of which is optionally substituted with from 1 to 4 substituents independently chosen from halogen, hydroxy, cyano, amino, —COOH and oxo;
R₅ is selected from the group consisting of methyl, ethyl, monofluoromethyl, difluoromethyl, trifluormethyl, difluoroethyl, methoxy, ethoxy, methoxymethyl, and CN;
R₆ is selected from the group consisting of hydrogen, halogen, CN, OR₇, amino, COOH, and C₁-C₄ alkyl, each of which is optionally substituted with from 1 to 4 substituents independently chosen from the group consisting of halogen, hydroxy, cyano, amino, —COOH and oxo; and
R₇ and R₈ are independently selected from among hydrogen, C₁-C₄ alkyl, C₂-C₄alkenyl, and C₂-C₄ alkynyl.

2. The compound of claim 1 wherein Ar is an optionally substituted heteroaryl group and the heteroaryl group is selected from the group consisting of pyridine, thiophene, thiazole, thiadiazole, furan, oxazole, oxadiazole, pyrrole, pyrazole, imidazole, triazole, oxathiole, isoxazole, oxazole, oxatriazole, dioxazole, oxathiazole, tetrazole, pyridazine, pyrimidine, pyrazine, triazine, oxazine, oxathiazine, and oxadiazine.

3. The compound of claim 2, wherein the heteroaryl group is substituted with a member of the group consisting of alkyl, alkoxy, trifluoroalkyl, trifluoroalkoxy, amino, halogen, hydroxyl, and CN, or forms an N-oxide.

4. The compound of claim 3, wherein Ar is an optionally substituted pyridine.

5. The compound of claim 3, wherein Ar is an optionally substituted pyrimidine group.

6. The compound of claim 3, wherein Ar is a phenyl group optionally substituted with CN, OCF₃ or halogen.

7. A compound according to claim 1, selected from the group consisting of

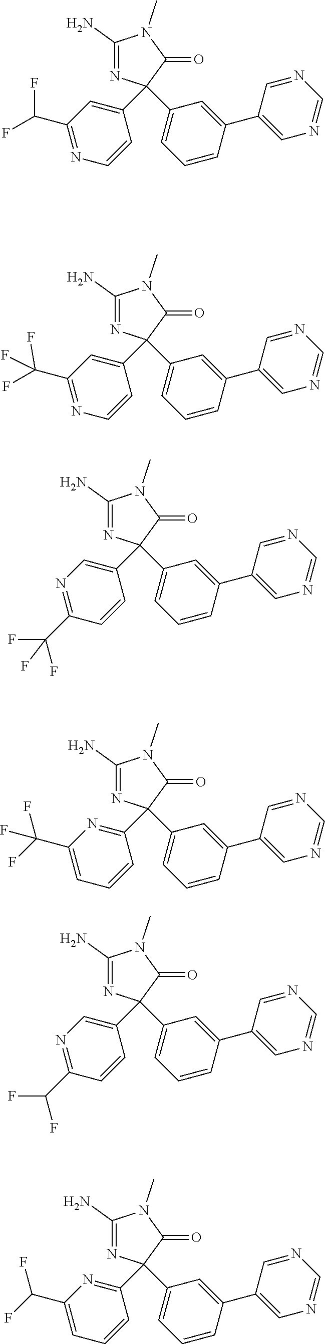

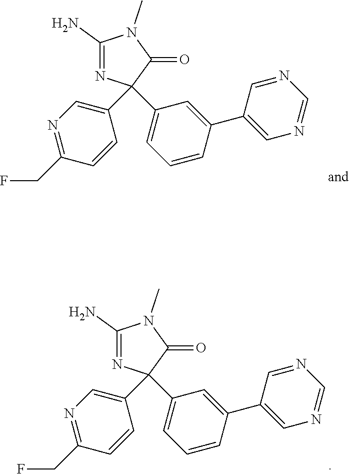 [SMILES: CN1C(=O)C(c2ccc(CF)nc2)(c2cccc(-c3cncnc3)c2)N=C1N ; CN1C(=O)C(c2ccc(CF)nc2)(c2cccc(-c3cncnc3)c2)N=C1N]

8. A composition comprising a compound of claim 1 and one or more pharmaceutically acceptable excipients.

9. A method of treating Alzheimer's disease, comprising administering an effective amount of a compound of claim 1 to a subject patient requiring such treatment.

10. A method of enhancing cognitive ability in a subject comprising administering an effective amount of a compound of claim 1 to subject in need thereof.

11. The method of claim 10, wherein the subject has Alzheimer's disease.

12. The method of claim 9 wherein the compound is administered orally.

13. The method of claim 10 wherein the compound is administered orally.
